(19) United States
(12) Patent Application Publication
(10) Pub. No.: US 2025/0120932 A1
(43) Pub. Date: Apr. 17, 2025

(54) PHARMACEUTICAL COMPOSITION

(71) Applicant: NEUROPRO THERAPEUTICS, INC., Yachats, OR (US)

(72) Inventor: Daryl W. HOCHMAN, Yachats, OR (US)

(21) Appl. No.: 18/922,269

(22) Filed: Oct. 21, 2024

Related U.S. Application Data
(63) Continuation of application No. 18/090,647, filed on Dec. 29, 2022, now abandoned.
(60) Provisional application No. 63/295,076, filed on Dec. 30, 2021.
(60) Provisional application No. 63/477,264, filed on Dec. 27, 2022.

(51) Int. Cl. A61K 31/196; A61K 9/00; A61K 9/08; A61K 9/48; A61K 47/10; A61K 47/22; A61K 47/32; A61K 47/34

(52) U.S. Cl. CPC A61K 31/196; A61K 9/0019; A61K 9/0043; A61K 9/0056; A61K 9/08; A61K 9/4858; A61K 9/4866; A61K 9/4875; A61K 47/10; A61K 47/22; A61K 47/32; A61K 47/34

(57) ABSTRACT

Described herein is Bumetanide Dibenzylamide, methods for synthesizing Bumetanide Dibenzylamide, pharmaceutical compositions thereof, and methods of dosing Bumetanide Dibenzylamide for treating epilepsy or other indication for which bumetanide is effective.

### CROSS-REFERENCE TO RELATED APPLICATIONS

The present application is a continuation of U.S. application Ser. No. 18/090,647, filed Dec. 29, 2022, which claims benefit of U.S. Provisional Application Ser. No. 63/477,264, filed Dec. 27, 2022, and U.S. Provisional Application Ser. No. 63/295,076, filed Dec. 30, 2021, each of which is herein incorporated by reference in their entirety.

### TECHNICAL FIELD

Described herein are compositions that comprise Bumetanide Dibenzylamide for treating selected conditions of the central and peripheral nervous systems employing non-synaptic mechanisms. More specifically, the present disclosure relates to methods and compositions for treating neurological disorders by administering agents that disrupt hypersynchronized neuronal activity without diminishing neuronal excitability. These compositions are useful for seizure disorders including epilepsy and related indications.

### BACKGROUND

Epilepsy is characterized by abnormal discharges of cerebral neurons and is typically manifested as various types of seizures. Many anti-convulsants originally developed for the treatment of epilepsy and other seizure disorders have also found application in the treatment of non-epileptic conditions, including neuropathic pain, mood disorders (such as bipolar affective disorder), and schizophrenia (for a review of the use of anti-epileptic drugs in the treatment of non-epileptic conditions, see Rogawski and Loscher, Nat. Medicine, 10:685-692, 2004). It has thus been suggested that epilepsy, neuropathic pain and affective disorders have a common pathophysiological mechanism (Rogawski & Loscher, ibid; Ruscheweyh & Sandkuhler, Pain 105:327-338, 2003), namely a pathological increase in neuronal excitability, with a corresponding inappropriately high frequency of spontaneous firing of neurons. However, only some, and not all, antiepileptic drugs are effective in treating neuropathic pain, and furthermore such antiepileptic drugs are only effective in certain subsets of patients with neuropathic pain (McCleane, Expert. Opin. Pharmacother. 5:1299-1312, 2004).

Epileptiform activity is identified with spontaneously occurring synchronized discharges of neuronal populations that can be measured using electrophysiological techniques. This synchronized activity, which distinguishes epileptiform from non-epileptiform activity, is referred to as “hypersynchronization” because it describes the state in which individual neurons become increasingly likely to dis-charge in a time-locked manner with one another. Hypersynchronized activity is typically induced in experimental models of epilepsy either by increasing excitatory or by decreasing inhibitory synaptic currents. It was therefore assumed that hyperexcitability per se was the defining feature involved in the generation and maintenance of epileptiform activity. Similarly, neuropathic pain was believed to involve conversion of neurons involved in pain transmission from a state of normal sensitivity to one of hypersensitivity (Costigan & Woolf, Jnl. Pain 1:35-44, 2000). The focus on developing treatments for both epilepsy and neuropathic pain has thus been on suppressing neuronal hyperexcitability by either: (a) suppressing action potential generation; (b) increasing inhibitory synaptic transmission, or (c) decreasing excitatory synaptic transmission.

Most agents currently used for treatment target synaptic activity in excitatory pathways by, for example, modulating the release or activity of excitatory neurotransmitters, potentiating inhibitory pathways, blocking ion channels involved in impulse generation, and/or acting as membrane stabilizers. Conventional agents and therapeutic approaches for the treatment of epilepsy and neuropsychiatric disorders thus reduce neuronal excitability and inhibit synaptic firing. One serious drawback of these therapies is that they are nonselective and exert their actions on both normal and abnormal neuronal populations. This leads to negative and unintended side effects, which may affect normal CNS functions, such as cognition, learning and memory, and produce adverse physiological and psychological effects in the treated patient. Common side effects include over-sedation, dizziness, loss of memory and liver damage. However, it has been shown that hypersychronous epileptiform activity can be dissociated from hyperexcitability and that the cation chloride cotransport inhibitor furosemide can reversibly block synchronized discharges without reducing hyperexcited synaptic responses (Hochman et al. Science 270:99-102, 1995).

The cation-chloride co-transporters (CCCs) are important regulators of neuronal chloride concentration that are believed to influence cell-to-cell communication, and various aspects of neuronal development, plasticity, and trauma. The CCC gene family consists of three broad groups: Na⁺—Cl⁻ co-transporters (NCCs), K⁺—Cl⁻ co-transporters (KCCs) and Na⁺—K⁺-2Cl⁻ co-transporters (NKCCs). Na—K—Cl co-transport in all cell and tissues is inhibited by loop diuretics, including furosemide, bumetanide, and benzmetanide. Espinosa et al. and Ahmad et al. have previously suggested that furosemide might be useful in the treatment of certain types of epilepsy (Medicina Espanola 61:280-281, 1969; and Brit. J. Clin. Pharmacol. 3:621-625, 1976). Bumetanide is potentially a more potent drug for treating epilepsy, but it also has a more pronounced diuretic effect. There is therefore a continuing need for methods and compositions for treating neuronal disorders that are not diuretic and that disrupt hypersynchronized neuronal activity without diminishing the neuronal excitability and spontaneous synchronization required for normal functioning of the peripheral and central nervous systems.

### SUMMARY

One embodiment of the present disclosure includes a pharmaceutical composition comprising Bumetanide Dibenzylamide and one or more solubilizers.

In one aspect, the composition comprises about 2.5 mg/mL to about 42 mg/mL of Bumetanide Dibenzylamide. In one aspect, the composition comprises about 0.25% w/w to about 15% w/w of Bumetanide Dibenzylamide. In one aspect, the composition comprises about 0.1% w/w to about 99.75% w/w of one or more solubilizers. In one aspect, the one or more solubilizers comprise short chain triglycerides, long chain triglycerides, or combinations thereof. In one aspect, the one or more solubilizers comprise Polyoxyl 35 Castor Oil, Glyceryl Monolinoleate, or any combination thereof. In one aspect, the one or more solubilizers comprise Caprylocaproyl Polyoxylglycerides, Phosphatidylcholine, Caprylic/Capric Triglyceride, Lauroyl Plyoxyl-32 Glycerides, Sorbitan Ester, or any combination thereof. In one aspect, the one or more solubilizers comprise Ethanol, Propylene Glycol, Polyethylene Glycol 600, Polyethylene Glycol 3350, Oleyl Alcohol, or any combination thereof. In one aspect, the one or more solubilizers comprise PEG-400, Vitamin E TPGS, or any combination thereof. In one aspect, the one or more solubilizers comprises soybean oil. In one aspect, the one or more solubilizers comprises water. In one aspect, the one or more solubilizers comprise Polyvinylpyrrolidone (K30), Poloxamer 407 (P407), Sodium Carboxymethyl cellulose (CMC), or any combination thereof. In one aspect, the one or more solubilizers comprises at least one super disintegrant. In one aspect, the one or more solubilizers comprises at least one wetting agents. In one aspect, the one or more solubilizers comprises at least one surfactants. In one aspect, the one or more solubilizers comprise Ceolus KG (microcrystalline cellulose), Mannogem EZ (spray dried mannitol), Polyplasdone XL (super disintegrate), Poloxamer 407, Lauroyl Plyoxyl-32 Glycerides, Sorbitan Ester, Neusilin US2 (magnesium aluminometasilicate), Citric acid Monohydrate, Cabosil M5P (fumed silica), Magnesium Stearate, or any combination thereof.

One embodiment of the present disclosure includes a pharmaceutical composition comprising about 1.79% w/w of Bumetanide Dibenzylamide, about 33.48% w/w of Polyoxyl 35 Castor oil, about 32.37% w/w of Glyceryl Monolinoleate, and about 32.37% w/w of Soybean oil.

One embodiment of the present disclosure includes a pharmaceutical composition comprising about 1.76% w/w of Bumetanide Dibenzylamide, about 32.93% w/w of Polyoxyl 35 Castor oil, about 31.83% w/w of Glyceryl Monolinoleate, about 31.83% w/w of Soybean oil, and about 10.37% w/w of Ethanol.

One embodiment of the present disclosure includes a pharmaceutical composition comprising about 1.59% w/w of Bumetanide Dibenzylamide, about 22% w/w of Phosphatidylcholine, about 70% w/w of Caprylocaproyl Polyoxylglycerides, and about 6.41% w/w of Caprylic/Capric Triglyceride.

One embodiment of the present disclosure includes a pharmaceutical composition comprising about 1.98% w/w of Bumetanide Dibenzylamide, about 19.39% w/w of Lauroyl Plyoxyl-32 Glycerides, about 37.62% w/w of Sorbitan Ester, and about 41.01% w/w of Soybean oil.

One embodiment of the present disclosure includes a pharmaceutical composition comprising about 1.98% w/w of Bumetanide Dibenzylamide, about 39.72% w/w of Caprylocaproyl Polyoxylglycerides, about 25.94% w/w of Sorbitan Ester, and about 49.78% w/w of Soybean oil.

One embodiment of the present disclosure includes a pharmaceutical composition comprising about 1.73% w/w of Bumetanide Dibenzylamide, about 32.40% w/w of Polyoxyl 35 Castor oil, about 31.32% w/w of Glyceryl Monolinoleate, about 31.32% w/w of Soybean oil, and about 3.24% w/w of Ethanol.

One embodiment of the present disclosure includes a pharmaceutical composition comprising about 1.7% w/w of Bumetanide Dibenzylamide, about 31.91% w/w of Polyoxyl 35 Castor oil, about 30.85% w/w of Glyceryl Monolinoleate, about 30.85% w/w of Soybean oil, and about 4.68% w/w of Ethanol.

One aspect of the present disclosure includes a pharmaceutical composition comprising about 10% w/w to about 100% w/w of Caprylocaproyl Polyoxylglycerides. One aspect of the present disclosure includes a pharmaceutical composition comprising about 12% w/w to about 20% w/w of Propylene Glycol. One aspect of the present disclosure includes a pharmaceutical composition comprising about 57% w/w to about 80% w/w of PEG 400. One aspect of the present disclosure includes a pharmaceutical composition comprising about 1% Vitamin E TPGS.

One embodiment of the present disclosure includes a pharmaceutical composition comprising about 2.73% w/w of Bumetanide Dibenzylamide, about 10% w/w of Caprylocaproyl Polyoxylglycerides, about 12.05% w/w of Propylene Glycol, about 67.22% w/w of Polyethylene Glycol 400, and about 8% w/w of Water.

One embodiment of the present disclosure includes a pharmaceutical composition comprising about 2.82% w/w of Bumetanide Dibenzylamide, about 4.65% w/w of Caprylocaproyl Polyoxylglycerides, about 13.01% w/w of Propylene Glycol, about 67.97% w/w of Polyethylene Glycol 400, about 0.92% w/w Polyvinylpyrrolidone (K30), and about 10.62% w/w of Water.

One embodiment of the present disclosure includes a pharmaceutical composition comprising about 0.6% w/w of Bumetanide Dibenzylamide, about 10% w/w of Caprylocaproyl Polyoxylglycerides, about 9.15% w/w of Propylene Glycol, about 53.84% w/w of Polyethylene Glycol 400, about 3.6% w/w of Polyvinylpyrrolidone (K30), about 2.4% w/w of Poloxamer 407 (P407), about 0.41% w/w Sodium CMC, and about 20% w/w of water.

One embodiment of the present disclosure includes a pharmaceutical composition comprising about 0.6% w/w of Bumetanide Dibenzylamide, about 10% w/w of Caprylocaproyl Polyoxylglycerides, about 9.15% w/w of Propylene Glycol, about 66.75% w/w of Polyethylene Glycol 400, about 3. % w/w of Polyvinylpyrrolidone (K30), about 2.4% w/w of Poloxamer 407 (P407), and about 7.5% w/w of Polyethylene Glycol 3350.

One embodiment of the present disclosure includes a pharmaceutical composition comprising about 15% w of Bumetanide Dibenzylamide, about 20% w of Microcrystalline Cellulose, about 51% w of Spray Dried Mannitol, about 7% w of Polyplasdone XL (super disintegrant), about 3% w of Poloxamer 407, about 1.5% w of Citric Acid Monohydrate, about 1.0% w of Cabosil M5P (fumed silica), and about 1.5% w of Magnesium Stearate.

One embodiment of the present disclosure includes a pharmaceutical composition comprising about 7.4% w of Bumetanide Dibenzylamide, about 9.9% w of Lauroyl Plyoxyl-32 Glycerides, about 9.9% w of Sorbitan Ester, about 54.3% w of Polyplasdone XL (super disintegrate), about 0.5% w of Poloxamer 407, about 0.7% w of Citric Acid Monohydrate, about 2.0% w of Cabosil M5P (fumed silica), about 14.8% w of Neusilin US2 (magnesium aluminometasilicate), and about 0.5% w of Magnesium Stearate.

One embodiment of the present disclosure includes a pharmaceutical composition comprising a vehicle composition comprising 55% Polyethylene Glycol 400 (PEG-400) in water, or 41% PEG-400, 12% Ethanol, 47% water or 45% PEG-400, 10% DMSO, 45% water, or 31% PEG-400, 31% Tetraglycol, 15% Caprylocaproyl Polyoxylglycerides, 23% water, or 30% PEG-400, 10% N-Methyl Pyrrolidone, 10% DMSO, 50% water, or 25% PEG-400, 10% DMSO, 20% Tetraglycol, 45% water, or 20% Ethanol in water, or 20% Hydroxypropyl-β-Cyclodextrin in water, or 11% DMSO, 22% PEG-400, 22% Tetraglycol, 44% water, or 44% PEG-400, 0.4% Poloxamer-188, 22% N-Methyl Pyrrolidone, 33% water, or 20% PEG-400, 15% Hydroxypropyl-β-Cyclodextrin in water, or 40% PEG-400, 20% Propylene Glycol, 5% Ethanol and 35%, water.

One embodiment of the present disclosure includes a pharmaceutical composition comprising about 28 g of Bumetanide Dibenzylamide, and about 3 ml of a solvent, wherein 50 g of solvent comprises about 5 g of Caprylocaproyl Polyoxylglycerides, about 10 g of Propylene Glycol, about 28.5 g of PEG-400, about 0.5 g of Vitamin E TPGS, about 1 g of Ethanol, and about 4.5 g of water.

One embodiment of the present disclosure includes a method for treating a patient in need thereof comprising: administering a pharmaceutical composition comprising Bumetanide Dibenzylamide and one or more solubilizers.

In an alternative embodiment, the present disclosure includes a pharmaceutical composition comprising Bumetanide Dibenzylamide and one or more solubilizers for use in medicine.

In a further alternative embodiment, there is provided use of a pharmaceutical composition comprising Bumetanide Dibenzylamide and one or more solubilizers in the manufacture of a medicament. In a further embodiment, a pharmaceutical composition comprising Bumetanide Dibenzylamide and one or more solubilizers may used of the manufacture of a medicament for the therapeutic and/or prophylactic treatment of seizure, epilepsy, and/or other indications such as neuropathic pain. In some embodiments, a pharmaceutical composition comprising Bumetanide Dibenzylamide and one or more solubilizers may used of the manufacture of a medicament for the therapeutic and/or prophylactic treatment of epilepsy and/or neurological syndromes that are specific to children. In some embodiments, a pharmaceutical composition comprising Bumetanide Dibenzylamide and one or more solubilizers may used of the manufacture of a medicament for the therapeutic and/or prophylactic treatment of one or more indications listed in FIG. 18 (Fisher et al.) and for treating whatever terms might replace the old and current terms describing the indications listed in FIG. 18 in the future. In some embodiments, a pharmaceutical composition comprising Bumetanide Dibenzylamide and one or more solubilizers may used of the manufacture of a medicament for the therapeutic and/or prophylactic treatment of migraines or tinnitus. In some embodiments, a pharmaceutical composition comprising Bumetanide Dibenzylamide and one or more solubilizers may used of the manufacture of a medicament for the therapeutic and/or prophylactic treatment of depression and/or anxiety. In some embodiments, a pharmaceutical composition comprising Bumetanide Dibenzylamide and one or more solubilizers may used of the manufacture of a medicament for the therapeutic and/or prophylactic treatment of psychoneurotic disorders. In some embodiments, a pharmaceutical composition comprising Bumetanide Dibenzylamide and one or more solubilizers may used of the manufacture of a medicament for the therapeutic and/or prophylactic treatment of neurodegenerative disorders including but not limited to Alzheimer's disease, amyotrophic lateral sclerosis, Friedrich ataxia, Huntington's Disease, Lewy Body disease, Parkinson's disease, or spinal muscular atrophy. In one aspect, the pharmaceutical composition comprises about 2.5 mg/mL to about 42 mg/mL of Bumetanide Dibenzylamide. In one aspect, the pharmaceutical composition comprises about 0.25% w/w to about 15% w/w of Bumetanide Dibenzylamide. In one aspect, the one or more solubilizers comprise short chain triglycerides, long chain triglycerides, or combinations thereof. In one aspect, the one or more solubilizers comprise polyoxyl 35 castor oil, Glyceryl Monolinoleate, or any combination thereof. In one aspect, the one or more solubilizers comprise Caprylocaproyl Polyoxylglycerides, Phosphatidylcholine, Caprylic/Capric Triglyceride, Lauroyl Plyoxyl-32 Glycerides, Sorbitan Ester or any combination thereof. In one aspect, the one or more solubilizers comprise Ethanol, Propylene Glycol, Polyethylene Glycol 600, Polyethylene Glycol 3350, Oleyl Alcohol, or any combination thereof. In one aspect, the one or more solubilizers comprise PEG-400, Vitamin E TPGS, or any combination thereof. In one aspect, the one or more solubilizers comprises Soybean oil. In one aspect, the one or more solubilizers comprises water. In one aspect, the one or more solubilizers comprise Polyvinylpyrrolidone (K30), Poloxamer 407 (P407), Sodium Carboxymethyl cellulose (CMC), or any combination thereof. In one aspect, the one or more solubilizers comprises at least one super disintegrant. In one aspect, the one or more solubilizers comprises at least one wetting agent. In one aspect, the one or more solubilizers comprises at least one surfactant. In one aspect, the one or more solubilizers comprise Ceolus KG (microcrystalline cellulose), Mannogem EZ (spray dried mannitol), Polyplasdone XL (super disintegrate), Poloxamer 407, lauroyl polyoxyl-32 glycerides, sorbitan ester, Neusilin US2 (magnesium aluminometasilicate), Citric acid Monohydrate, Cabosil M5P (fumed silica), Magnesium Stearate or any combination thereof. In one aspect, the pharmaceutical composition comprising about 1.79% w/w of Bumetanide Dibenzylamide, about 33.48% w/w of Polyoxyl 35 Castor oil, about 32.37% w/w of Glyceryl Monolinoleate, and about 32.37% w/w of Soybean oil. In one aspect, the pharmaceutical composition comprising about 1.76% w/w of Bumetanide Dibenzylamide, about 32.93% w/w of Polyoxyl 35 Castor oil, about 31.83% w/w of Glyceryl Monolinoleate, about 31.83% w/w of Soybean oil, and about 10.37% w/w of Ethanol. In one aspect, the pharmaceutical composition comprising about 1.59% w/w of Bumetanide Dibenzylamide, about 22% w/w of Phosphatidylcholine, about 70% w/w of Caprylocaproyl Polyoxylglycerides, and about 6.41% w/w of Caprylic/Capric Triglyceride. In one aspect, the pharmaceutical composition comprising about 1.98% w/w of Bumetanide Dibenzylamide, about 19.39% w/w of Lauroyl Plyoxyl-32 Glycerides, about 37.62% w/w of Sorbitan Ester, and about 41.01% w/w of Soybean oil. In one aspect, the pharmaceutical composition comprising about 1.98% w/w of Bumetanide Dibenzylamide, about 39.72% w/w of Caprylocaproyl Polyoxylglycerides, about 25.94% w/w of Sorbitan Ester, and about 49.78% w/w of Soybean oil. In one aspect, the pharmaceutical composition comprising about 1.73% w/w of Bumetanide Dibenzylamide, about 32.40% w/w of Polyoxyl 35 Castor oil, about 31.32% w/w of Glyceryl Monolinoleate, about 31.32% w/w of Soybean oil, and about 3.24% w/w of Ethanol. In one aspect, the pharmaceutical composition comprising about 1.7% w/w of Bumetanide Dibenzylamide, about 31.91% w/w of Polyoxyl 35 Castor oil, about 30.85% w/w of Glyceryl Monolinoleate, about 30.85% w/w of Soybean oil, and about 4.68% w/w of Ethanol. In one aspect, the pharmaceutical composition comprising about 10% w/w to about 100% w/w of Caprylocaproyl Polyoxylglycerides. In one aspect, the pharmaceutical composition comprising about 12% w/w to about 20% w/w of Propylene Glycol. In one aspect, the pharmaceutical composition comprising about 57% w/w to about 80% w/w of PEG 400. In one aspect, the pharmaceutical composition comprising about 1% Vitamin E TPGS. In one aspect, the pharmaceutical composition comprising about 2.73% w/w of Bumetanide Dibenzylamide, about 10% w/w of Caprylocaproyl Polyoxylglycerides, about 12.05% w/w of Propylene Glycol, about 67.22% w/w of Polyethylene Glycol 400, and about 8% w/w of Water. In one aspect, the pharmaceutical composition comprising about 2.82% w/w of Bumetanide Dibenzylamide, about 4.65% w/w of Caprylocaproyl Polyoxylglycerides, about 13.01% w/w of Propylene Glycol, about 67.97% w/w of Polyethylene Glycol 400, about 0.92% w/w Polyvinylpyrrolidone (K30), and about 10.62% w/w of Water. In one aspect, the pharmaceutical composition comprising about 0.6% w/w of Bumetanide Dibenzylamide, about 10% w/w of Caprylocaproyl Polyoxylglycerides, about 9.15% w/w of Propylene Glycol, about 53.84% w/w of Polyethylene Glycol 400, about 3.6% w/w of Polyvinylpyrrolidone (K30), about 2.4% w/w of Poloxamer 407 (P407), about 0.41% w/w Sodium CMC, and about 20% w/w of water. In one aspect, the pharmaceutical composition comprising about 0.6% w/w of Bumetanide Dibenzylamide, about 10% w/w of Caprylocaproyl Polyoxylglycerides, about 9.15% w/w of Propylene Glycol, about 66.75% w/w of Polyethylene Glycol 400, about 3.6% w/w of Polyvinylpyrrolidone (K30), about 2.4% w/w of Poloxamer 407 (P407), and about 7.5% w/w of Polyethylene Glycol 3350. In one aspect, the pharmaceutical composition comprising about 15% w of Bumetanide Dibenzylamide, about 20% w of Microcrystalline Cellulose, about 51% w of Spray Dried Mannitol, about 7% w of Polyplasdone XL (super disintegrate), about 3% w of Poloxamer 407, about 1.5% w of Citric Acid Monohydrate, about 1.0% w of Cabosil M5P (fumed silica), and about 1.5% w of Magnesium Stearate. In one aspect, the pharmaceutical composition comprising about 7.4% w of Bumetanide Dibenzylamide, about 9.9% w of Lauroyl Plyoxyl-32 Glycerides, about 9.9% w of Sorbitan Ester, about 54.3% w of Polyplasdone XL (super disintegrate), about 0.5% w of Poloxamer 407, about 0.7% w of Citric Acid Monohydrate, about 2.0% w of Cabosil M5P (fumed silica), about 14.8% w of Neusilin US2 (magnesium aluminometasilicate), and about 0.5% w of Magnesium Stearate. In one aspect, the pharmaceutical composition comprising a vehicle composition comprising 55 Polyethylene Glycol 400 (PEG-400) in water, or 41% PEG-400, 12% ethanol, 47% water or 45% PEG-400, 10% DMSO, 45% water, or 31% PEG-400, 31% Tetraglycol, 15% Caprylocaproyl Polyoxylglycerides, 23% water, or 30% PEG-400, 10% N-Methyl Pyrrolidone, 10% DMSO, 50% water, or 25% PEG-400, 10% DMSO, 20% Tetraglycol, 45% water, or 20% Ethanol in water, or 20% Hydroxypropyl-β-Cyclodextrin in water, or 11% DMSO, 22% PEG-400, 22% Tetraglycol, 44% water, or 44% PEG-400, 0.4% Poloxamer-188, 22% N-Methyl pyrrolidone, 33% water, or 20% PEG-400, 15% Hydroxypropyl-β-Cyclodextrin in water, or 40% PEG-400, 20% Propylene Glycol, 5% Ethanol, and 35% water. In one aspect, the pharmaceutical composition comprising about 28 g of Bumetanide Dibenzylamide, and about 3 ml of a solvent, wherein 50 g of solvent comprises about 5 g of Caprylocaproyl Polyoxylglycerides, about 10 g of Propylene Glycol, about 28.5 g of PEG-400, about 0.5 g of Vitamin E TPGS, about 1 g of Ethanol, and about 4.5 g of water. In one aspect, the pharmaceutical composition is an oral capsule. In one aspect, the pharmaceutical composition is a nasal solution. In one aspect, the pharmaceutical composition is a rectal gel. In one aspect, the pharmaceutical composition is a rectal paste. In one aspect, the pharmaceutical composition is a sublingual tablet. In one aspect, the pharmaceutical composition is an injectable composition. In one aspect, the dosing of the patient with the pharmaceutical composition is done orally. In one aspect, the dosing of the patient with the pharmaceutical composition is done intranasally. In one aspect, the dosing of the patient with the pharmaceutical composition is done rectally. In one aspect, the dosing of the patient with the pharmaceutical composition is done sublingually. In one aspect, the dosing of the patient with the pharmaceutical composition is done subcutaneously. In one aspect, the dosing of the patient with the pharmaceutical composition is done intramuscularly.

One embodiment of the present disclosure includes a pharmaceutical composition comprising Bumetanide Dibenzylamide and one or more organic anion transport (OAT) inhibitors. The role of active transport in tissue distribution of bumetanide has been extensively studied for the kidney and the liver but not the BBB. In mice and humans, OATs are thought to mediate the renal transport of bumetanide. These transporters maybe essentially related to the transport of bumetanide on the luminal and basolateral membrane site of the kidney proximal tubular cells. Bumetanide seems not to be transported by OATPs in kidney or liver, although only few members of this transporter family have been studied in this respect. Probenecid inhibits OATs which explains that it has been shown to reduce the plasma and renal clearance of bumetanide in dogs. In line with these observations in dogs, probenecid markedly increased the plasma half-life of bumetanide in mice. However, it did not decrease the diuretic effect of bumetanide. Systemic administration of an organic anion transport inhibitor may have a beneficial effect on brain levels of bumetanide or products thereof. For background teaching, see Tollner et al., European Journal of Pharmacology, 746 (2015) 167-173, herein incorporated by reference with regard to such teaching.

In one aspect, the one or more OAT inhibitors are competitive antagonists. In one aspect, the Bumetanide Dibenzylamide and the one or more OAT inhibitors are formulated with different release profiles. In one aspect, the at least one OAT inhibitor is formulated to be released prior to release of the Bumetanide Dibenzylamide. In one aspect, the at least one OAT inhibitor is formulated to be released prior to the Bumetanide Dibenzylamide achieving maximum plasma concentration (Cₘₐₓ) in a patient receiving the composition. In one aspect, wherein the one or more OAT inhibitor is selected from the group consisting of probenecid, aspirin, ibuprofen, acetylsalicylic acid, diclofenac, aspartame, and valproic acid.

One embodiment of the present disclosure includes a pharmaceutical composition comprising Bumetanide Dibenzylamide in a self-emulsifying drug delivery system (SEDDS). In one aspect, the SEDDS comprises an isotropic mixture of oils, solubilizers, surfactants, and co-solvents.

One embodiment of the present disclosure includes a pharmaceutical composition comprising Bumetanide Dibenzylamide in an oral lymphatic targeted formulation.

In one aspect, the composition comprises Bumetanide Dibenzylamide, Polyoxyl 35 Castor Oil (Kolliphor EL), Glyceryl Monolinoleate (Maisine CC), Soybean Oil, and Ethanol. In one aspect, the composition comprises about 1 to 2 w/w % Bumetanide Dibenzylamide, about 30 to 35 w/w % Polyoxyl 35 Castor Oil (Kolliphor EL), about 30 to 35 w/w % Glyceryl Monolinoleate (Maisine CC), about 30 to 35 w/w % Soybean Oil, and about 2.5 to 5 w/w % Ethanol. In one aspect, the composition comprises about 1.7 w/w % Bumetanide Dibenzylamide, about 32.4 w/w % Polyoxyl 35 Castor Oil (Kolliphor EL), about 31.3 w/w % Glyceryl Monolinoleate (Maisine CC), about 31.3 w/w % Soybean Oil, and about 3.2 w/w % Ethanol.

One embodiment of the present disclosure includes a pharmaceutical composition comprising a prodrug of Bumetanide and one or more oral lymphatic targeting excipients. A prodrug of Bumetanide may include any compound comprising Bumetanide including but not limited to amide prodrug forms, which have been demonstrated to offer one or more unexpected benefits over other prodrug forms.

In one aspect, the prodrug of Bumetanide is an amide prodrug. In one aspect, the prodrug of Bumetanide is one or more of Bumetanide Dibenzylamide, Bumetanide Diethylamide, and Bumetanide Morpholinoamide. In one aspect the composition comprises alkoxylated castor oil. In one aspect the composition comprises Polyoxyl 35 Castor Oil (Kolliphor EL). In one aspect the composition comprises one or more of a mono-, di-, and triglyceride. In one aspect the composition comprises Glyceryl Monolinoleate (Maisine CC). In one aspect the composition comprises a fixed oil. In one aspect, the composition comprises soybean oil. In one aspect the composition comprises a water soluble solvent. In one aspect, the composition comprises ethanol.

For the avoidance of doubt, the prodrugs of Bumetanide described herein (e.g. Bumetanide Dibenzylamide, Bumetanide Diethylamide, and Bumetanide Morpholinoamide), and compositions comprising prodrugs of Bumetanide, may be suitable and/or preferred treatment agents for use in the methods of the present disclosure (including all aspects and embodiments of those methods). The prodrugs of Bumetanide may be used to treat epilepsy, Alzheimer, and other diseases and indication comprising seizure.

In one aspect, the pharmaceutical composition comprises up to 17.5% w of Bumetanide Dibenzylamide, about 20% w solubilizer, and about 15% w absorbent.

Throughout the present disclosure, any description of a method may be interpreted to describe and support a commensurate use, manufacture for use, composition, composition for use, or other alternative description.

One or more embodiments or aspects may be incorporated in a different embodiment or aspect although not specifically described. That is, all embodiments and aspects can be combined in any way or combination.

### BRIEF DESCRIPTION OF THE DRAWINGS

FIG. 1A shows the individual animal Bumetanide Dibenzylamide concentration-time profiles following IN administration of 15 mg Bumetanide Dibenzylamide to male and female Dogs on a linear scale.

FIG. 1B shows the individual animal Bumetanide Dibenzylamide concentration-time profiles following IN administration of 15 mg Bumetanide Dibenzylamide to male and female Dogs on a log-linear scale.

FIG. 2A shows the individual animal Bumetanide Dibenzylamide concentration-time profiles following SL administration of 30 mg Bumetanide Dibenzylamide to male and female Dogs on a linear scale.

FIG. 2B shows the individual animal Bumetanide Dibenzylamide concentration-time profiles following SL administration of 30 mg Bumetanide Dibenzylamide to male and female Dogs on a log-linear scale.

FIG. 3A shows the individual animal Bumetanide Dibenzylamide concentration-time profiles following IM administration of 15 mg Bumetanide Dibenzylamide to male and female Dogs on a linear scale.

FIG. 3B shows the individual animal Bumetanide Dibenzylamide concentration-time profiles following IM administration of 15 mg Bumetanide Dibenzylamide to male and female Dogs on a log-linear scale.

FIG. 4A shows the individual animal Bumetanide Dibenzylamide concentration-time profiles following PO administration of 30 mg Bumetanide Dibenzylamide to male and female Dogs on a linear scale.

FIG. 4B shows the individual animal Bumetanide Dibenzylamide concentration-time profiles following PO administration of 30 mg Bumetanide Dibenzylamide to male and female Dogs on a log-linear scale.

FIG. 5A shows the individual animal Bumetanide Dibenzylamide concentration-time profiles following SC administration of 15 mg Bumetanide Dibenzylamide to male and female Dogs on a linear scale.

FIG. 5B shows the individual animal Bumetanide Dibenzylamide concentration-time profiles following SC administration of 15 mg Bumetanide Dibenzylamide to male and female Dogs on a log-linear scale.

FIG. 6A shows the individual animal Bumetanide Dibenzylamide concentration-time profiles following IR administration of 30 mg Bumetanide Dibenzylamide to male and female Dogs on a linear scale.

FIG. 6B shows the individual animal Bumetanide Dibenzylamide concentration-time profiles following IR administration of 30 mg Bumetanide Dibenzylamide to male and female Dogs on a log-linear scale.

FIG. 7A shows the mean Bumetanide Dibenzylamide concentration-time profiles following various routes of administration of 30 mg Bumetanide Dibenzylamide to male and female dogs on a linear scale.

FIG. 7B shows the mean Bumetanide Dibenzylamide concentration-time profiles following various routes of administration of 30 mg Bumetanide Dibenzylamide to male and female dogs on a log-linear scale.

FIG. 8A shows the mean Bumetanide Dibenzylamide and Bumetanide plasma concentration-time profiles following IV bolus administration of 10 mg/kg Bumetanide Dibenzylamide to male rats on a linear scale.

FIG. 8B shows the mean Bumetanide Dibenzylamide and Bumetanide plasma concentration-time profiles following IV bolus administration of 10 mg/kg Bumetanide Dibenzylamide to male rats on a semi-log scale.

FIG. 9A shows the mean Bumetanide Dibenzylamide and Bumetanide plasma concentration-time profiles following oral administration of 30 mg/kg Bumetanide Dibenzylamide to male rats on a liner scale.

FIG. 9B shows the mean Bumetanide Dibenzylamide and Bumetanide plasma concentration-time profiles following oral administration of 30 mg/kg Bumetanide Dibenzylamide to male rats on a semi-log scale.

FIG. 10A shows the mean Bumetanide Dibenzylamide and Bumetanide plasma concentration-time profiles following IV (10 mg/kg) and oral (30 mg/kg) doses Bumetanide Dibenzylamide to male rats on a liner scale.

FIG. 10B shows the mean Bumetanide Dibenzylamide and Bumetanide plasma concentration-time profiles following IV (10 mg/kg) and oral (30 mg/kg) doses Bumetanide Dibenzylamide to male rats on a semi-log scale.

FIG. 11A shows the Bumetanide plasma concentration-time profiles following oral administration of 30 mg/kg Bumetanide Dibenzylamide to dogs on a liner scale.

FIG. 11B shows the Bumetanide plasma concentration-time profiles following oral administration of 30 mg/kg Bumetanide Dibenzylamide to dogs a semi-log scale.

FIG. 12 shows the X-ray diffractogram analysis of bumetanide dibenzylamide.

FIG. 13 shows the DSC analysis of bumetanide dibenzylamide.

FIG. 14 shows the TGA analysis of bumetanide dibenzylamide.

FIG. 15 shows the DVS analysis of bumetanide dibenzylamide.

FIG. 16 shows the PSD analysis of bumetanide dibenzylamide.

FIG. 17 shows the morphology of Bumetanide Dibenzylamide under 40× magnification.

FIG. 18 is a listing of indications, in order to reference variability in classification over time.

FIG. 19 is a tabulated calculation of the Permeability of Test Compounds in Caco-2 Assay of Example 9.

FIG. 20 is a tabulated depiction of LCMS data from the Permeability of Test Compounds in Caco-2 Assay of Example 9.

FIG. 21 is a tabulated depiction of the Lucifer Yellow Rejection Assay for Monolayer Integrity Test associated with the Permeability of Test Compounds in Caco-2 Assay of Example 9.

FIGS. 22A, 22B, and 22C likewise are (A) a tabulated calculation of the Permeability of Test Compounds in Caco-2 Assay of Example 9; (B) a tabulated depiction of LCMS data from the Permeability of Test Compounds in Caco-2 Assay of Example 9; and (C) a tabulated depiction of the Lucifer Yellow Rejection Assay for Monolayer Integrity Test associated with the Permeability of Test Compounds in Caco-2 Assay of Example 9.

FIG. 23 shows the blood concentration of the formulated (black triangles) and unformulated (grey circles) Bumetanide Morpholinoamide at 2 hrs, 6 hrs, and 12 hrs post treatment.

FIG. 24 illustrates formulated and unformulated Bumetanide Morpholinoamide blood concentration time points prior to 2 hrs using an exponential fit to the data.

FIG. 25 shows the blood concentration of the formulated (black triangles) and unformulated (grey circles) Bumetanide Diethylamide at 2 hrs, 6 hrs, and 12 hrs post treatment.

### DETAILED DESCRIPTION

The terms “active ingredient”, “active pharmaceutical ingredient,” and “API” as used herein refer to a pharmaceutical agent, active ingredient, compound, or substance, compositions, or mixtures thereof, that provide a pharmacological, often beneficial, effect.

The term “dose” as used herein denotes any form of the active ingredient formulation that contains an amount sufficient to produce a therapeutic effect with a single administration.

The term “dosage” as used herein refers to the administering of a specific amount, number, and frequency of doses over a specified period-of-time, typically one (1) day.

The terms “active pharmaceutical ingredient load” or “drug load” as used herein refers to the quantity (mass) of the active pharmaceutical ingredient comprised in a single soft capsule fill.

The terms “formulation” or “pharmaceutical composition” or “composition” as used herein refers to the drug in combination with pharmaceutically acceptable excipients.

The term mean “particle size distribution” (PSD) as used herein refers to the mean particle size from a statistical distribution of a range of particle sizes as described herein. The distribution may be a Gaussian, normal distribution, or a non-normal distribution.

The terms such as “d90,” “d50,” and “d10” refer to the percentage (e.g., 90%, 50%, or 10%, respectively) of particle sizes that are less than a specified size, range, or distribution. For example, “d90≤100 μm” as means that 90% of the particle sizes within a distribution of particles are less than or equal to 100 μm.

As used herein, the term “patient” refers to any subject including mammals and humans. The patient may have a disease or suspected of having a disease and as such is being treated with a drug. In some instances, the patient is a mammal, such as a human, non-human primate, dog, cat, horse, cow, goat, pig, rabbit, rat, mouse, or a premature neonate, neonate, infant, juvenile, adolescent, or adult thereof. In some instances, the term “patient,” as used herein, refers to a human (e.g., a man, a woman, or a child). In some instances, the term “patient,” as used herein, refers to laboratory animal of an animal model study. The patient or subject may be of any age, sex, or combination thereof.

The terms “biological sample” or “sample” as used herein refers to a sample obtained or derived from a patient. By way of example, a biological sample comprises a material selected from the group consisting of body fluids, blood, whole blood, plasma, serum, mucus secretions, saliva, cerebrospinal fluid (CSF), bronchoalveolar lavage fluid (BALF), urine, fluids of the eye (e.g., vitreous fluid, aqueous humor), lymph fluid, lymph node tissue, spleen tissue, bone marrow, and fluid from the auditory cavity.

The term “treating” refers to administering a therapy in an amount, manner, or mode effective (e.g., a therapeutic effect) to improve a condition, symptom, disorder, or parameter associated with a disorder, or a likelihood thereof.

The term “prophylaxis” refers to preventing or reducing the progression of a disorder, either to a statistically significant degree or to a degree detectable to one skilled in the art.

The terms “essentially” or “substantially” as used herein mean to a great or significant extent, but not completely.

The term “about” as used herein refers to any values, including both integers and fractional components that are within a variation of up to +10% of the value modified by the term “about.”

Also described herein are pharmaceutical compositions and dosage forms comprising one or more agents that reduce the rate by which the compositions described herein as active ingredients will decompose. Such agents, which are referred to herein as “stabilizers,” include, but are not limited to, antioxidants such as ascorbic acid, pH buffers, salts, sugars, etc.

The phrase ‘solubilizer’ is used to refer to an ingredient or group of ingredients that helps solubilize the composition or part of the composition.

The phrases and terms “can be administered by injection”, “injectable”, or “injectability” refer to a combination of factors such as a certain force applied to a plunger of a syringe containing the formulations described herein and at a certain temperature, a needle of a given inner diameter connected to the outlet of such syringe, and the time required to extrude a certain volume of the Bumetanide Dibenzylamide composition from the syringe through the needle.

The range for each ingredient in the described formulation represent the space in which a suitable alternative(s) may be obtained in combination with the other ingredients in ratios adjusted to total 100% w/w. The ranges provided are estimates based on the available data.

One embodiment described herein, is a pharmaceutical composition comprising Bumetanide Dibenzylamide. In one aspect, the composition comprises any of the formulations shown in the Tables or Examples described herein. Any of the components in the formulations described herein, shown in the Tables, or illustrated in the Examples can be increased, decreased, combined, substituted, or omitted to provide for a formulation comprising about 100% by weight. Such compositions are hereby disclosed as if they were expressly disclosed herein.

One embodiment described herein is a pharmaceutical composition comprising Bumetanide Dibenzylamide, and one or more solubilizers. Another embodiment described herein is a pharmaceutical composition comprising Bumetanide Dibenzylamide. Another embodiment described herein is a pharmaceutical composition further comprising one or more additional solvents. Another embodiment described herein is a pharmaceutical composition further comprising one or more surfactants, co-surfactants, emulsifying agent or wetting agent. Another embodiment described herein is a pharmaceutical composition consisting essentially of Bumetanide Dibenzylamide. Another embodiment described herein is a pharmaceutical composition consisting essentially of aqueous Bumetanide Dibenzylamide. Another embodiment described herein is a pharmaceutical composition comprising Bumetanide Dibenzylamide, and one or more solubilizers. Another embodiment described herein is a pharmaceutical composition consisting essentially of Bumetanide Dibenzylamide, and one or more solubilizers. In one aspect, the composition is a dry powder compressed into a tablet. In one aspect, the composition is a dry powder filled into a capsule. In one aspect, the composition is a dry powder extruded into a film. In one aspect, the composition is a dry powder extruded into a tablet. One embodiment described herein is a pharmaceutical composition comprising about 2.5 mg to about 42 mg of bumetanide dibenzylamide.

One embodiment described herein is a pharmaceutical composition formulated as an oral capsule. In one aspect, the composition comprises up to about 0.25% w/w to about 15% w/w of Bumetanide Dibenzylamide, and one or more solubilizers. In one aspect, the solubilizer is a co-solvent. In one aspect, the solubilizer is a surfactant. In one aspect, the solubilizer comprises triglycerides. In one aspect, the triglycerides comprises medium chain triglycerides. In one aspect, the triglycerides comprises long chain triglycerides. In one aspect, the triglycerides comprises a mixture of medium and long chain triglycerides. In one aspect, the triglycerides comprises polyoxylglycerides. In one aspect, the polyoxylglycerides is selected from a group consisting Lauroyl Polyoxylglycerides, Linoleoyl Polyoxylglycerides, Oleoyl Polyoxylglycerides, Stearoyl Polyoxylglycerides, Caprylocaproyl Polyoxylglyceride, and any combination thereof. In one aspect, the triglyceride comprises a non-ionic surfactant, solubilizer, emulsifying agent. In one aspect, the long chain triglycerides are selected from a group consisting of Polyoxyl 35 Castor Oil (Kolliphor EL), Glyceryl Monolinoleate (Maisine CC), and any combination thereof. In one aspect, the medium chain triglycerides are selected from a group consisting of Caprylocaproyl Polyoxylglycerides (Labrasol ALF), Phosphatidylcholine (Phosal 75 SA), Caprylic/Capric Triglyceride (Captex 300), Lauroyl Plyoxyl-32 Glycerides (Gelucire 44/14), Sorbitan Ester (Span 80), and any combination thereof.

In one aspect, described herein is a pharmaceutical composition comprising about 0.1% w/w to about 99.75% w/w of one or more solubilizers. In one aspect, the solubilizer comprises soybean oil. In one aspect, the solubilizer is in the oil phase. In one aspect, the solubilizer is selected from a group consisting of Arachis oil, soybean oil, castor oil, corn oil, safflower oil, olive oil, apricot kernel oil, sesame oil, cotton-seed oil, sunflower seed oil, palm oil and rapeseed oil, Maisine 35-1, Maisine CC (Glyceryl Monolinoleate), and any combination thereof. In one aspect, the solubilizer comprises a co-solvent. In one aspect, the solubilizer is selected from a group consisting of Propylene Glycol, Capryol™ 90 (Propylene glycol monocaprylate), Lauroglycol™ 90 (Propylene glycol monolaurate), Glycerin, Polyethylene Glycol, and any combination thereof. In one aspect, the solubilizer comprises an antioxidant. In one aspect, the solubilizer is selected from a group consisting of alpha tocopherol, ascorbyl palmitate, ascorbic acid, butylated hydroxyanisole, butylated hydroxytoluene, and any combination thereof. In one aspect, the solubilizer comprises an antimicrobial preservative, a solvent, and a water-soluble co-solvent. In one aspect, the solubilizer comprises a solvent, and a water-soluble co-solvent. In one aspect, the solubilizer is selected from a group consisting Ethanol, Propylene Glycol, Propylene Glycol 300, Propylene Glycol 400, Propylene Glycol 600, Oleyl Alcohol, and any combination thereof. In one aspect, the solubilizer is water. In one aspect, the solubilizer is any diluent.

In one aspect, described herein is a pharmaceutical composition comprising about 0.5% w/w to about 1.8% w/w of Bumetanide Dibenzylamide. In one aspect, described herein is a pharmaceutical composition comprising about 9 mg of Bumetanide Dibenzylamide per capsule to about 12 mg of Bumetanide Dibenzylamide per capsule. In one aspect, described herein is a pharmaceutical composition comprising about 0% w/w to about 1.8% w/w of Bumetanide Dibenzylamide, about 10% w/w to about 45% w/w of Polyoxyl 35 Castor Oil (Kolliphor EL), about 15% w/w to about 65% w/w of Glyceryl Monolinoleate (Maisine CC), about 15% w/w to about 65% w/w of Soybean Oil, about 0% w/w to about 15% w/w of Ethanol, and about 0% w/w to about 0.13% w/w of Butylated Hydroxytoluene. In one aspect, described herein is a pharmaceutical composition comprising about 1.75% w/w of Bumetanide Dibenzylamide, about 32.37% w/w of Polyoxyl 35 Castor Oil (Kolliphor EL), about 31.30% w/w of Glyceryl Monolinoleate (Maisine CC), about 31.30% w/w of Soybean Oil, about 3.25% w/w of Ethanol, and about 0.3% w/w of Butylated Hydroxytoluene.

One embodiment described herein is a pharmaceutical composition formulated as a nasal solution. In one aspect, the composition comprises Bumetanide Dibenzylamide in a solvent system. In one aspect, the composition comprises about 3 ml of solvent and from about 28 mg of Bumetanide Dibenzylamide to about 32 mg of Bumetanide Dibenzylamide. In one aspect, the solubilizer comprises triglycerides. In one aspect, the triglycerides comprises medium chain triglycerides. In one aspect, the triglycerides comprises long chain triglycerides. In one aspect, the triglycerides comprises a mixture of medium and long chain triglycerides. In one aspect, the triglycerides comprises polyoxylglycerides. In one aspect, the polyoxylglycerides is selected from a group consisting Lauroyl Polyoxylglycerides, Linoleoyl Polyoxylglycerides, Oleoyl Polyoxylglycerides, Stearoyl Polyoxylglycerides, Caprylocaproyl Polyoxylglyceride, and any combination thereof.

In one aspect, the triglyceride comprises a non-ionic surfactant, solubilizer, emulsifying agent. In one aspect, the solubilizer comprises Caprylocaproyl Polyoxylglycerides (Labrasol ALF). In one aspect, the solvent system comprises one or more solubilizers. In one aspect, the solubilizer comprises Caprylocaproyl Polyoxylglycerides (Labrasol ALF), and water. In one aspect, the solubilizer comprises Caprylocaproyl Polyoxylglycerides (Labrasol ALF), Propylene Glycol, and water. In one aspect, the solubilizer comprises Caprylocaproyl Polyoxylglycerides (Labrasol ALF), Propylene Glycol, PEG-400, and water. In one aspect, the solubilizer comprises Caprylocaproyl Polyoxylglycerides (Labrasol ALF), Propylene Glycol, PEG-400, Vitamin E TPGS, and water. In one aspect, the solubilizer comprises Caprylocaproyl Polyoxylglycerides (Labrasol ALF), Propylene Glycol, PEG-400, Vitamin E TPGS, Ethanol, and water. In one aspect, the solubilizer comprises about 50 g of Caprylocaproyl Polyoxylglycerides (Labrasol ALF), per 50 g of solvent. In one aspect, the solubilizer comprises about 25 g of Caprylocaproyl Polyoxylglycerides (Labrasol ALF), and about 25 g of water per 50 g of solvent. In one aspect, the solubilizer comprises about 6 g of Propylene Glycol, about 40 g of PEG-400, and about 4 g of water per 50 g of solvent. In one aspect, the solubilizer comprises about 5 g of Caprylocaproyl Polyoxylglycerides (Labrasol ALF), about 6 g of Propylene Glycol, about 35 g of PEG-400, and about 4 g of water per 50 g of solvent. In one aspect, the solubilizer comprises about 10 g of Propylene Glycol, about 35 g or PEG-400, about 0.5 g of Vitamin E TPGS, and about 4.5 g of water per 50 g of solvent. In one aspect, the solubilizer comprises about 5 g of Caprylocaproyl Polyoxylglycerides (Labrasol ALF), about 10 g of Propylene Glycol, about 28.5 g of PEG-400, about 0.5 g of Vitamin E TPGS, about 1 g of Ethanol, and about 4.5 g of water per 50 g of solubilizer. In one aspect, the solubilizer comprises glycofurol. In one aspect, the solubilizer comprises a penetration agent, and a solvent. In one aspect, the solubilizer comprises ethyl oleate. In one aspect, the solubilizer comprises an oleaginous vehicle, solvent, and a solvent.

In one aspect, the pharmaceutical composition comprises about 3% w/v of Bumetanide Dibenzylamide, about 11% w/w of Caprylocaproyl Polyoxylglycerides (Labrasol ALF), about 13.26% w/v of Propylene Glycol, about 73.94% w/v of PEG-400, and about 8.8% w/w of water. In one aspect, the pharmaceutical composition comprises from about 0.01% w/w to about 40% w/w of Bumetanide Dibenzylamide, from about 5% w/w to about 100% w/w of Caprylocaproyl Polyoxylglycerides (Labrasol ALF), from about 4% w/w to about 20% w/w of Propylene Glycol, from about 50% w/w to about 80% w/w of PEG-400, and from about 0% w/w to about 10% w/w of water. In one aspect, the pharmaceutical composition comprises about 2.73% w/w of Bumetanide Dibenzylamide, about 8% w/w of Caprylocaproyl Polyoxylglycerides (Labrasol ALF), about 15% w/w of Propylene Glycol, about 69.27% w/w of PEG-400, and about 5% w/w of water. In one aspect, the pharmaceutical composition comprises about 2.73% w/w of Bumetanide Dibenzylamide, about 16% w/w of Caprylocaproyl Polyoxylglycerides (Labrasol ALF), about 20% w/w of Propylene Glycol, about 54.27% w/w of PEG-400, and about 7% w/w of water. In one aspect, the pharmaceutical composition comprises about 2.73% w/w of Bumetanide Dibenzylamide, about 5% w/w of Caprylocaproyl Polyoxylglycerides (Labrasol ALF), about 4% w/w of Propylene Glycol, about 78.27% w/w of PEG-400, and about 10% w/w of water.

One embodiment described herein is a pharmaceutical composition formulated as a rectal paste. One embodiment described herein is a composition formulated as a rectal gel. In one aspect, the composition is formulated with a target of about 6 mg Bumetanide Dibenzylamide per gram of the composition based on a target dose of about 30 mg of Bumetanide Dibenzylamide in an amount of about 5 g of the composition. In one aspect, the composition is formulated with a different target dose of Bumetanide Dibenzylamide. In one aspect, the paste is determined to be a 100% non-aqueous formulation in case the drug substance exhibited some instability in water. In one aspect, the rectal gel is formulated to comprise about 0.6% w/w Bumetanide Dibenzylamide, about 10% w/w Caprylocaproyl Polyoxylglycerides (Labrasol ALF), about 9.15% w/w Propylene Glycol, about 53.84% w/w Polyethylene Glycol 600, about 3.6% w/w Polyvinylpyrrolidone (K30), about 2.4% w/w Poloxamer 407 (P407), about 0.41% w/w Sodium Carboxymethyl Cellulose (CMC), and about 20% w/w water. In one aspect, the rectal paste is formulated to comprise about 0.6% w/w Bumetanide Dibenzylamide, about 10% w/w Caprylocaproyl Polyoxylglycerides (Labrasol ALF), about 9.15% w/w Propylene Glycol, about 66.75% w/w Polyethylene Glycol 600, about 3.6% w/w Polyvinylpyrrolidone (K30), about 2.4% w/w Poloxamer 407 (P407), and about 7.5% w/w Polyethylene Glycol 3350.

One embodiment described herein is a pharmaceutical composition formulated as a sublingual tablet. In one aspect, the composition formulated is targeted to have about 30 mg Bumetanide Dibenzylamide per tablet. In one aspect, a small tablet size is used. In one aspect, wetting and or dissolving of the composition occurs within 30 seconds. In one embodiment, the sublingual tablet is formulated to comprise Bumetanide Dibenzylamide, one or more wetting agent, and one or more super disintegrate. In one aspect, the sublingual tablet is formulated to comprise about 15% w Bumetanide Dibenzylamide, about 20% w Ceolus KG (microcrystalline cellulose), about 51% w Mannogem EZ (spray dried mannitol), about 7% w Polyplasdone XL (super disintegrate), about 3% w Poloxamer 407 (wetting agent), about 1.5% w Citric Acid Monohydrate, about 1% w Cabosil M5P (fumed silica), and about 1.5% w Magnesium Stearate. In one embodiment, the sublingual tablet is formulated to comprise Bumetanide Dibenzylamide, one or more water dispersible surfactant, one or more wetting agent, and one or more super disintegrate. In one aspect, the sublingual tablet is formulated to comprise about 7.4% w Bumetanide Dibenzylamide, about 9.9% w Lauroyl Plyoxyl-32 Glycerides (Gelucire 44/14, a water dispersible surfactant), about 9.9% w Sorbitan Ester (Span 80, a water dispersible surfactant), about 14.8% w Neusilin US2 (Magnesium Aluminometasilicate), about 0.5% w/w Poloxamer 407 (wetting agent), about 0.7% w Citric Acid Monohydrate, about 2% w Cabosil M5P (Fumed Silica), about 54.3% w Polyplasdone XL (Super Disintegrate), and about 0.5% w Magnesium Stearate.

One embodiment described herein, the preferred treatment agents and methods of the present disclosure (including all aspects and embodiments of those agents and methods) are for use in treating seizures (e.g., partial onset seizures), epilepsy, and/or other indications such as neuropathic pain by modulating or disrupting the synchrony of neuronal population activity in areas of heightened synchronization by reducing the activity of NKCC co-transporters without having a diuretic effect. One embodiment described herein, are preferred treatment agents and methods of the present disclosure for treating seizures that cannot be controlled by existing pharmacotherapeutics such as uncontrolled seizures, intractable seizures, refractory seizures, drug resistant seizures, or medically resistant seizures. One embodiment described herein, are preferred treatment agents and methods of the present disclosure for treating epilepsy syndromes such as Angelman syndrome, benign rolandic epilepsy, CDKL5 disorder, childhood absence epilepsy, Dravet syndrome, GLUT1 deficiency syndrome, hypothalamic hamartoma, infantile spasms (also known as West syndrome), Lennox-Gastaut, PCDH19, progressive myoclonic epilepsy, Rasmussen's encephalitis, ring chromosome 20 syndrome, or reflex epilepsies.

One embodiment described herein, the preferred treatment agents and methods of the present disclosure (including all aspects and embodiments of those agents and methods) are for use in treating the epilepsy and/or neurological syndromes that are specific to children including but not limited to Dravett syndrome, infantile spasms, Landau-Kleffner Syndrome, Lennox-Gastaut Syndrome, Rasmussen Syndrome, Benign Rolandic Epilepsy, Benign Occipital Epilepsy, Childhood Absence Epilepsy, Juvenile Myoclonic, Rett Syndrome, Angelman Syndrome, Tuberous Sclerosis, and/or Sturge Weber Syndrome. One embodiment described herein, the preferred treatment agents and methods of the present disclosure are for use in treating epilepsy and/or neurological syndromes that may be observed in adults or children.

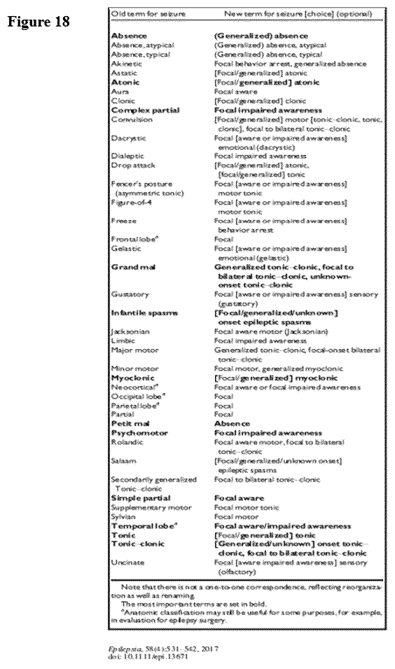

One embodiment described herein, are preferred treatment agents and methods of the present disclosure (including all aspects and embodiments of those agents and methods) for use in treating one or more indications listed in FIG. 18 (Fisher et al.) and for treating whatever terms might replace the old and current terms describing the indications listed in FIG. 18 in the future.

One embodiment described herein, are preferred treatment agents and methods of the present disclosure (including all aspects and embodiments of those agents and methods) for use in treating comorbidities of epilepsy or seizures such as psychiatric disorders such as depression, anxiety disorders, attention deficit hyperactivity disorder (ADHD), schizophrenia-like interictal psychosis, autism, as well as suicidal behavior, sleep disorders, autism spectrum disorders, migraines, postictal headaches, depression, anxiety, psychosis, Attention Deficit Disorder (ADD) and Attention Deficit/Hyperactivity Disorder (ADHD), or mental retardation.

One embodiment described herein, are preferred treatment agents and methods of the present disclosure (including all aspects and embodiments of those agents and methods) for use in treating migraines or tinnitus. In one aspect, the preferred treatment agents and methods of the present disclosure may be used to treat migraines with or without aura in adults. In another aspect, the preferred treatment agents and methods of the present disclosure may be used for acute treatment of migraine with auro, acute treatment of migraine without auro, or for chronic treatment for the prevention of migraine with our without aura.

One embodiment described herein, are preferred treatment agents and methods of the present disclosure (including all aspects and embodiments of those agents and methods) for use in treating mild, moderate, or severe anxiety. In one aspect, the preferred treatment agents and methods of the present disclosure may be used for the acute and maintenance treatment of Major Depressive disorder (MDD) in adults and adolescents aged 12-17 years, or for the acute treatment of Generalized Anxiety Disorder (GAD) in adults. In one aspect, the preferred treatment agents and methods of the present disclosure may be used for the acute and maintenance treatment of Obsessive Compulsive Disorder (OCD), acute and maintenance treatment of Bulimia Nervosa, or acute treatment of Panic Disorder (PD) with or without agoraphobia. In another aspect, the preferred treatment agents and methods of the present disclosure may be used for treating acute depressive episodes associated with bipolar I disorder or for treating treatment resistant depression.

One embodiment described herein, are preferred treatment agents and methods of the present disclosure (including all aspects and embodiments of those agents and methods) may be for use in treating obsessions and compulsions in patients with Obsessive Compulsive Disorder (OCD), Major Depressive Disorder (MDD), Panic Disorder (PD), Social Anxiety Disorder (SAD), Pre-menstrual dysphoric disorder (PMDD), or Posttraumatic Stress Disorder (PTSD). In one aspect, the obsessions or compulsions may cause marked distress, be time-consuming, or significantly interfere with social or occupational functioning, in order to meet the DSM-III-R (circa 1989) diagnosis of OCD. Obsessions may be recurrent, persistent ideas, thoughts, images, or impulses that are egodystonic. Compulsions may be repetitive, purposeful, and/or intentional behaviors performed in response to an obsession or performed in a stereotyped fashion. Compulsions may be recognized by the person as excessive or unreasonable

One embodiment described herein, are preferred treatment agents and methods of the present disclosure (including all aspects and embodiments of those agents and methods) for use in treating psychoneurotic patients with mild, moderate, or severe depression, anxiety associated with depression, anxiety as associated with alcoholism, depression and/or anxiety associated with organic disease, psychotic depressive disorders with associated anxiety including involutional depression and manic-depressive disorders. In one aspect, the preferred treatment agents and methods of the present disclosure may be used to target symptoms of psychoneurosis such as anxiety, tension, depression, somatic symptoms and concerns, sleep disturbances, guilt, lack of energy, fear, apprehension, and worry.

One embodiment described herein, are preferred treatment agents and methods of the present disclosure (including all aspects and embodiments of those agents and methods) for use in treating depressive illness in patients with depressive neurosis (dysthymic disorder), manic-depressive illness, or with major depressive disorder. In one aspect, the preferred treatment agents and methods of the present disclosure may be used for short-term, long-term and maintenance treatment of Major Depressive Disorder (MDD), Generalized Anxiety Disorder, Diabetic Peripheral Neuropathic Pain (DPNP), Fibromyalgia (FM), or Chronic Musculoskeletal Pain.

One embodiment described herein, are treatment agents and methods of the present disclosure for treating a progressive neurodegenerative disorder, including, for example, Alzheimer's disease. In one aspect, the preferred treatment agents and methods of the present disclosure may be used for treatment or cessation of disease progression for one or more of Alzheimer's disease, amyotrophic lateral sclerosis, Friedrich ataxia, Huntington's disease, Lewy Body disease, Parkinson's disease, or spinal muscular atrophy.

One embodiment described herein, are preferred treatment agents and methods of the present disclosure comprising use as a monotherapy or an adjunctive therapy. In one aspect, the preferred treatment agents and methods of the present disclosure may be used as a monotherapy for adults. In another aspect, the preferred treatment agents and methods of the present disclosure may be used as an adjunctive therapy with additional therapy agents for adults. In another aspect, the preferred treatment agents and methods of the present disclosure may be used as a monotherapy for pediatric patients 2 years of age and older. In another aspect, the preferred treatment agents and methods of the present disclosure may be used as an adjunctive therapy with additional therapy agents for pediatric patients 2 years of age and older. In another aspect, the preferred treatment agents and methods of the present disclosure may be used as a monotherapy for pediatric patients less than 2 years of age. In another aspect, the preferred treatment agents and methods of the present disclosure may be used as an adjunctive therapy with additional therapy agents for pediatric patients less than 2 years of age.

One embodiment described herein, are preferred treatment agents and methods of the present disclosure (including all aspects and embodiments of those agents and methods) comprising use as a monotherapy or an adjunctive therapy. In one aspect, the preferred treatment agents and methods of the present disclosure may be used as a monotherapy for adults. In another aspect, the preferred treatment agents and methods of the present disclosure may be used as an adjunctive therapy with additional therapy agents for adults. In another aspect, the preferred treatment agents and methods of the present disclosure may be used as a monotherapy for pediatric patients 2 years of age and older. In another aspect, the preferred treatment agents and methods of the present disclosure may be used as an adjunctive therapy with additional therapy agents for pediatric patients 2 years of age and older. In another aspect, the preferred treatment agents and methods of the present disclosure may be used as a monotherapy for pediatric patients less than 2 years of age. In another aspect, the preferred treatment agents and methods of the present disclosure may be used as an adjunctive therapy with additional therapy agents for pediatric patients less than 2 years of age.

The effective amount of an active pharmaceutical ingredient to be administered therapeutically will depend, for example, upon the therapeutic context and objectives. One having ordinary skill in the art will appreciate that the appropriate dosage levels for treatment will vary depending, in part, upon the concentration of the Bumetanide Dibenzylamide composition (or a composition comprising any other prodrug of Bumetanide described herein), the dosing regimen for which the Bumetanide Dibenzylamide composition (or a composition comprising any other prodrug of Bumetanide described herein) is being used, the route of administration, and the subject's size (body weight or body surface area) and condition (the age and general health) of the patient. Accordingly, the dosage may be tittered to obtain the optimal therapeutic effect.

The frequency of dosing will depend upon the pharmacokinetic parameters of the therapeutic agent incorporated into the Bumetanide Dibenzylamide composition (or a composition comprising any other prodrug of Bumetanide described herein) being used. The composition can be administered as a single dose, as two or more doses (which may or may not contain the same amount of the Bumetanide Dibenzylamide, or any other prodrug of Bumetanide described herein) over time, or as a continuous infusion of an injection formulation via an implantation device or catheter. Further refinement of the appropriate dosage is routinely made by those of ordinary skill in the art and is within the ambit of tasks routinely performed by them. A sublingual tablet may also be used for oral administration). Appropriate dosages can be ascertained through use of appropriate dose-response data.

Refinement opportunities may include extended or controlled release oral capsule or tablet or use of a transdermal formulation. Intramuscular data shown below supports the development of a potential transdermal therapy. The intramuscular data shows that Bumetanide Dibenzylamide or any other prodrug of Bumetanide described herein can be absorbed through the microvessels of the muscle into the circulation, and thus avoiding first-pass metabolism. Hence the Bumetanide Dibenzylamide should also be absorbed by the dermal microvessels, lending itself to a transdermal formulation.

The Bumetanide Dibenzylamide composition (or a composition comprising any other prodrug of Bumetanide described herein) can be administered, for example, 1×, 2×, 3×, 4×, 5×, 6×, or even more times per day. One or more doses can be administered, for example, for 1, 2, 3, 4, 5, 6, 7 days, or even longer. One or more doses can be administered, for example, for 1, 2, 3, 4 weeks, or even longer. One or more doses can be administered, for example, for 1, 2, 3, 4, 5, 6, 7, 8, 9, 10, 11, 12 months, 1 year, 2, years, 3 years, 4 years, 5 years, over 5 years, a decade, multiple decades, or even longer. One or more doses can be administered at a regular interval until the subject or subject in need thereof, does not require treatment or prophylaxis of epilepsy. In one aspect, the doses maybe administered orally. In one aspect, the doses maybe administered sublingually. In one aspect, the doses maybe administered intravenously. In one aspect, the doses maybe administered intrarectally. In one aspect, the doses maybe administered intramuscularly. In one aspect, the doses maybe administered intranasally. In one aspect, the doses maybe administered subcutaneously.

In one embodiment, the pharmaceutical composition described herein is administered in one or multiple doses simultaneously. For example, two or more identical doses are administered at one time. In another embodiment, two or more different doses are administered at one time. Such dual or different simultaneous doses can be used to provide an effective amount of the pharmaceutical composition to a subject in need thereof.

In one embodiment, the pharmaceutical compositions described herein may be used to treat, prevent, retard the progression of, delay the onset, ameliorate, reduce the symptoms of, or prophylaxis of epilepsy.

In one embodiment, the Bumetanide Dibenzylamide composition (or a composition comprising any other prodrug of Bumetanide described herein) described herein is sufficiently dosed in the composition to provide a therapeutically effective amount in one application. In one aspect, one application of Bumetanide Dibenzylamide composition (or a composition comprising any other prodrug of Bumetanide described herein) is sufficient for about 1 day, about 2 days, about 3 days, about 4 days, about 5 days, about 1 week, about 2 weeks, about 3 weeks, about 4 weeks, one month, 2 months, 3 months, 4 months, 6 months, 9 months, one year, 2 years, 3 years, 4 years, or even longer. In one aspect, one application of Bumetanide Dibenzylamide composition (or a composition comprising any other prodrug of Bumetanide described herein) is given more than once per day.

Another embodiment, the Bumetanide Dibenzylamide composition, (or a composition comprising any other prodrug of Bumetanide described herein) described herein is provided as a single dose, meaning that the container in which it is supplied contains one pharmaceutical dose. In another embodiment, the composition is provided as a multiple dose composition, meaning that it contains more than one therapeutic dose. Preferably, a multiple dose composition contains at least 2 doses. Such multiple dose Bumetanide Dibenzylamide composition (or a composition comprising any other prodrug of Bumetanide described herein) either can be used for different subjects in need thereof or is intended for use in one subject, wherein the remaining doses are stored after the application of the first dose until needed. In another embodiment, the Bumetanide Dibenzylamide composition (or a composition comprising any other prodrug of Bumetanide described herein) is comprised in one or more containers.

Crucial to the efficacy of any treatment by a pharmaceutical composition is the overall systemic bioavailability of the pharmaceutical composition used for said treatment. Surprisingly, lipid formulations may be used to increase the bioavailability and/or lymphatic absorption of a pharmaceutical composition. LogP is one measure of lipophyilicity, and is the octal:water partition coefficient expressed as a log ratio of molecule in octanol relative to water after mixing. The LogP of Bumetanide is about 2.61, the logP of Bumetanide Diethylamide is 3.11, and the logP of Bumetanide Dibenzylamide is about 5.9. A LogP of 3 indicates a 1000-fold greater concentration in octanal than water, so Bumetanide Dibenzyl amide is about 1000—to about 10,000-fold more lipophilic than Bumetanide.

As described herein, it is found that Bumetanide Dibenzylamide or any other prodrug of Bumetanide described herein disrupts the synchrony of neuronal population activity in areas of heightened synchronization. The dosage compositions of Bumetanide Dibenzylamide were developed targeting four (4) routes of administrations in attempt to bypass first-pass metabolism for of the composition and increase overall systemic bioavailability. The routes of administration were chosen based upon their potential to maximize the bioavailability of Bumetanide Dibenzylamide and to produce measurable systemic concentrations of Bumetanide Dibenzylamide. Since Bumetanide Dibenzylamide has been shown to be susceptible to high first-pass metabolism by the liver, the routes of administration were selected to avoid hepatic metabolism.

As described below, the pharmacokinetic profiles and bioavailability of the composition were evaluated following oral (PO), sublingual (SL), intranasal (IN), intrarectal (IR), subcutaneous (SC), intravenous (IV), and intramuscular (IM) dose administration. It was surprisingly found that oral administration of Bumetanide Dibenzylamide formulated with triglycerides bypasses first-pass metabolism of the composition by the liver.

In one embodiment described herein, the pharmacokinetic profiles and bioavailability of Bumetanide Dibenzylamide following oral (PO), sublingual (SL), intranasal (IN), intrarectal (IR), subcutaneous (SC), and intramuscular (IM) dose administration is evaluated in a crossover design in male and female Beagle dogs. The IN, IM, and SC dose level is about 15 mg of Bumetanide Dibenzylamide and drug exposure is the highest following IM administration followed by similar exposures for SC and IN administration. The PO, SL, and IR dose level is about 30 mg of Bumetanide Dibenzylamide and the drug exposure is much higher for PO administration followed by IR administration. The Bumetanide Dibenzylamide concentration following SL administration is relatively low.

### Study Design

Male and female Beagle dogs are assigned to two groups with 2 males and 2 females in each group. The study is conducted in 3 legs with Leg 1 consisting of IM and SL administration, Leg 2 consisting of IN and PO administration, and Leg 3 consisting of SC and IR administration. As illustrated in Table 1, group 1 animals receive IM, IN and SC dosing, and Group 2 animals receive SL, PO, and IR administration, each in a crossover design. Group 1 animals receive about 15 mg doses and Group 2 receive about 30 mg doses of Bumetanide Dibenzylamide.

TABLE 1
Group Assignments and Treatment Information

| | | | | | Dose | Dose | Dose |
|---|---|---|---|---|---|---|---|
| Leg | Group | Dose | N | | Level | Concentration | Volume |
| # | # | Route | M | F | (mg) | (mg/mL) | (mL) |
| 1 | 1 | IM | 2 | 2 | 15 | 30 | 0.5 |
| | 2 | SL | 2 | 2 | 30 | NA | 1 tablet |
| 2 | 1 | IN | 2 | 2 | 15 | 30 | 0.5* |
| | 2 | PO | 2 | 2 | 30 | NA | 3 capsules |
| 3 | 1 | SC | 2 | 2 | 15 | 30 | 0.5 |
| | 2 | IR | 2 | 2 | 30 | 60 | 0.5 |

### Dosing Procedures

Administration of Bumetanide Dibenzylamide composition is as described herein. During intranasal administration, the composition is administered by gently tilting the animals head back and the intranasal dose is delivered via a syringe and atomizer. During sublingual administration, the composition is administered via a quick dissolving tablet. The tablet is placed in the sublingual space and the mouth held shut for approximately 60-90 seconds. After one minute, the mouth of the animal is opened to confirm that the tablet has fully dissolved. During intramuscular administration, the composition is administered into the bicep femoris (rear thigh). The dose site is gently shaved prior to dosing. A syringe with a 25 g needle is utilized. During oral administration, the composition is administered via three capsules placed at the back of the animal's mouth and the placement is followed by a 10 mL drinking water flush to assure that the animal swallowed the capsules. During subcutaneous administration, the composition is administered into the dorsal subcutaneous space directly at the scruff of the neck of the animal. The dose site is gently shaved prior to dosing. A syringe with a 25 g needle is utilized. During intrarectal administration, the composition is administered by gently placing a 1 cc syringe in the rectum of the animal.

All animals are fasted for approximately 12 hours prior to dosing. Animals are fed four hours post dosing with water offered ad libitum throughout the study. All animals completed the study. Body weights remained stable and no adverse reactions are noted throughout the study with all animals showing normal behavior following dose administrations. Blood and urine samples are collected out to 24 hours. Table 2 contains information regarding the sex and body weights of the animals in the study.

TABLE 2
Sex and Body Weights (kg) of Study Animals

| Animal # | | Sex | Leg 1 | Leg 2 | Leg 3 |
|---|---|---|---|---|---|
| 2960045 | (#1) | Male | 13.2 | 13.5 | 13.5 |
| 2923344 | (#2) | Male | 13.5 | 13.5 | 13.3 |
| CJDCDD | (#3) | Female | 12.0 | 12.0 | 11.3 |
| CJCCJW | (#4) | Female | 11.4 | 11.2 | 11.0 |
| 2972702 | (#5) | Male | 12.2 | 13.0 | 12.5 |
| 2974674 | (#6) | Male | 13.1 | 13.1 | 13.1 |
| CJACUH | (#7) | Female | 8.7 | 8.3 | 8.3 |
| CJCCBU | (#8) | Female | 10.0 | 9.4 | 9.5 |

### Plasma Sample Analysis for Bumetanide Dibenzylamide and Bumetanide

Blood sample are collected from the jugular vein, or other suitable vessel via direct venipuncture, placed into a chilled tube containing K2EDTA as the anticoagulant, and inverted several times to mix. Blood samples are kept on wet ice until centrifugation. Plasma is harvested and stored on dry ice until placed in a freezer, set to maintain 80° C., until analysis.

Plasma samples are analyzed for Bumetanide Dibenzylamide and Bumetanide concentration using a LC-MS/MS method. The assay is verified with respect to standard bioanalytical methodology including acceptable accuracy and precision based upon quality control sample analysis and results. The range of the method for both analytes is from about 0.250 ng/ml to about 250 ng/mL.

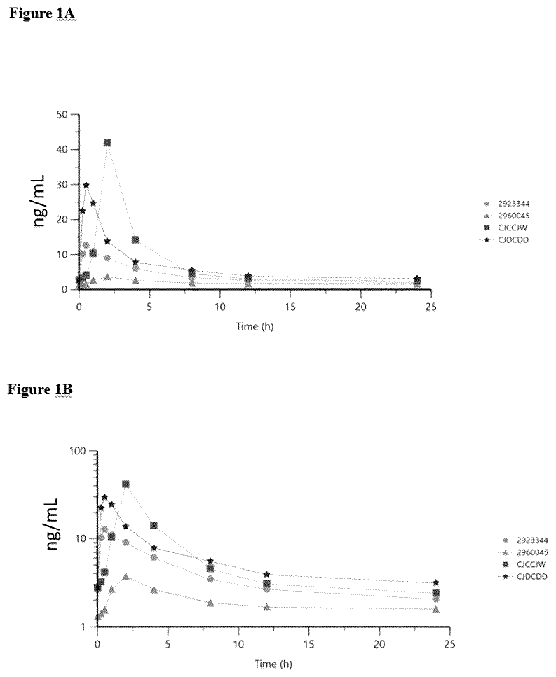

Plasma Bumetanide Dibenzylamide concentration-time data are summarized for all routes of administration in Tables 3-8. Individual animal data are plotted by formulation group on linear and log-linear axes in FIGS. 1-6.

Plasma concentration data following IN administration of about 15 mg dose are displayed in Table 3 and plotted in FIGS. 1A-1B. Bumetanide Dibenzylamide concentrations are variable between dogs during the first 2 hours after dosing with % CV ranging from 75.5 to 105.9%. The variability then decreased until the % CV was 28.5% at 24 hours. Each of the female dogs displayed higher concentrations than the 2 male dogs.

TABLE 3
Summary of Plasma Bumetanide Dibenzylamide Concentration-Time Data in Dogs
Following Administration of a Single Dose of an Intranasal (IN) Formulation

| | | | Time (h) | | | | | | | | |
|---|---|---|---|---|---|---|---|---|---|---|---|
| | Dose | | 0 | 0.25 | 0.5 | 1.0 | 2.0 | 4.0 | 8.0 | 12.0 | 24.0 |
| Route | (mg) | Animal | Bumetanide Dibenzylamide (ng/mL) | | | | | | | | |
| IN | 15 | 2923344 | 2.63 | 10.2 | 12.7 | 10.9 | 9.05 | 6.08 | 3.47 | 2.68 | 2.07 |
| | | 2960045 | 1.32 | 1.40 | 1.55 | 2.68 | 3.73 | 2.65 | 1.88 | 1.68 | 1.59 |
| | | CJCCJW | 2.79 | 3.24 | 4.14 | 10.3 | 41.9 | 14.2 | 4.57 | 3.08 | 2.43 |
| | | CJDCDD | 2.79 | 22.5 | 29.8 | 24.7 | 13.8 | 7.83 | 5.55 | 3.92 | 3.15 |
| | | Mean | 2.38 | 9.34 | 12.0 | 12.1 | 17.1 | 7.69 | 3.87 | 2.84 | 2.31 |
| | | CV % | 29.9 | 102.4 | 105.9 | 75.5 | 99.4 | 63.0 | 40.7 | 32.7 | 28.5 |

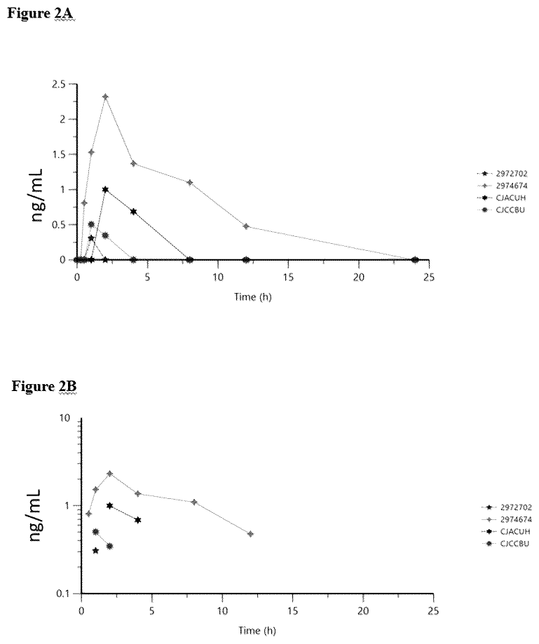

Plasma concentration data following SL administration of about 30 mg dose is displayed in Table 4 and plotted in FIGS. 2A-2B. SL administration provided the lowest concentrations of any of the 6 formulations. Bumetanide Dibenzylamide is absorbed slowly with the first measurable concentration occurring at 0.5 h after dosing in one of the dogs. All dogs have measurable concentrations at 2 hours after dosing. Concentrations are variable through the 24-hour sampling period with % CV ranging from 111.7 to 200%. For the SL route of administration, each of the male dogs displayed higher concentrations than the 2 female dogs.

TABLE 4
Summary of Plasma Bumetanide Dibenzylamide Concentration-Time Data in Dogs
Following Administration of a Single Dose of a Sublingual (SL) Formulation

| | | | Time (h) | | | | | | | | |
|---|---|---|---|---|---|---|---|---|---|---|---|
| | Dose | | 0.00 | 0.25 | 0.50 | 1.00 | 2.00 | 4.00 | 8.00 | 12.00 | 24.00 |
| Route | (mg) | Animal | Bumetanide Dibenzylamide (ng/mL) | | | | | | | | |
| SL | 30 | 2972702 | 0.00 | 0.00 | 0.00 | 0.308 | 0.00 | 0.00 | 0.00 | 0.00 | 0.00 |
| | | 2974674 | 0.00 | 0.00 | 0.809 | 1.53 | 2.32 | 1.37 | 1.10 | 0.477 | 0.00 |
| | | CJACUH | 0.00 | 0.00 | 0.00 | 0.00 | 1.00 | 0.686 | 0.00 | 0.00 | 0.00 |
| | | CJCCBU | 0.00 | 0.00 | 0.00 | 0.505 | 0.346 | 0.00 | 0.00 | 0.00 | 0.00 |
| | | Mean | 0.00 | 0.00 | 0.202 | 0.586 | 0.917 | 0.514 | 0.275 | 0.119 | 0.00 |
| | | CV % | | | 200.0 | 113.2 | 111.7 | 127.6 | 200.0 | 200.0 | |

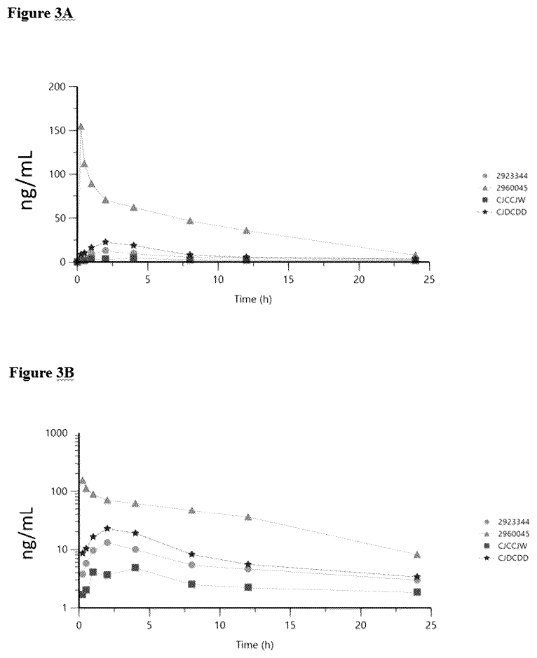

The results following administration of a 15 mg IM dose is illustrated in Table 5 and plotted in FIGS. 3A-3C. One of the male dogs (animal #1) displayed very high concentrations, beginning at 0.25 h after dosing, that continued throughout the sampling period, suggesting that the dose may have been inadvertently injected into a vein. The Tmax for the remaining dogs occurred at 2 to 4 hours indicating slower absorption from the IM injection site.

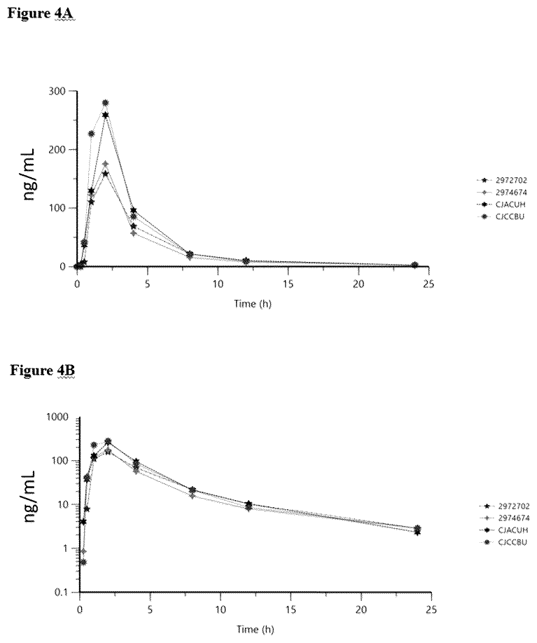

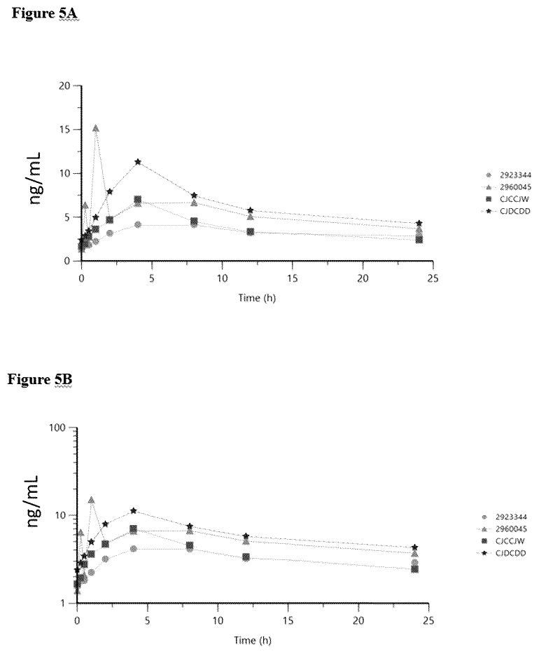

Plasma concentration data following PO administration of a 30 mg dose is illustrated in Table 6 and plotted in FIGS. 4A-4B. The Bumetanide Dibenzylamide Tmax is at 2 hours after dosing in all 4 animals and the concentrations for all are consistently higher than by any of the other routes of administration. In addition, the variability is lower than for the other formulations and decreased over time, from 51.2% at 0.5 hour to just 9.9% at 24 hours. For the PO route of administration, each of the female dogs displayed higher concentrations than the 2 male dogs.

The results following SC administration of a 15 mg dose is illustrated in Table 7 and plotted in FIGS. 5A-5B. Bumetanide Dibenzylamide concentrations were relatively consistent between dogs throughout the 24-hour sampling period but never reached concentrations one might expect following SC administration. One animal (animal #1) displayed a high concentration of 15.2 ng/mL at 1 hour after dosing that appeared to be an outlier based on the concentrations in nearby samples. With the exception of that 1 hour sample the variability in the remainder of the data is relatively low.

TABLE 5
Summary of Plasma Bumetanide Dibenzylamide Concentration-Time Data in Dogs Following
Administration of a Single Dose of an Intramuscular (IM) Formulation

| | | | Time (h) | | | | | | | | |
|---|---|---|---|---|---|---|---|---|---|---|---|
| | Dose | | 0.00 | 0.25 | 0.50 | 1.00 | 2.00 | 4.00 | 8.00 | 12.00 | 24.00 |
| Route | (mg) | Animal | Bumetanide Dibenzylamide (ng/mL) | | | | | | | | |
| IM | 15 | 2923344 | 0.00 | 3.77 | 5.77 | 9.58 | 13.1 | 10.0 | 5.43 | 4.63 | 2.99 |
| | | 2960045 | 0.00 | 155 | 112 | 89.3 | 70.9 | 62.4 | 47.2 | 36.2 | 8.23 |
| | | CJCCJW | 0.00 | 1.70 | 2.01 | 4.06 | 3.66 | 4.87 | 2.54 | 2.24 | 1.87 |
| | | CJDCDD | 0.00 | 8.59 | 10.3 | 16.4 | 22.7 | 18.9 | 8.20 | 5.54 | 3.40 |
| | | Mean | 0.00 | 42.3 | 32.5 | 29.8 | 27.6 | 24.0 | 15.8 | 12.2 | 4.12 |
| | | CV % | | 178.0 | 163.3 | 133.9 | 108.4 | 109.1 | 132.8 | 132.4 | 68.3 |

TABLE 6
Summary of Plasma Bumetanide Dibenzylamide Concentration-Time Data in Dogs
Following Administration of a Single Dose of an Oral (PO) Formulation

| | | | Time (h) | | | | | | | | |
|---|---|---|---|---|---|---|---|---|---|---|---|
| | Dose | | 0.00 | 0.25 | 0.50 | 1.00 | 2.00 | 4.00 | 8.00 | 12.00 | 24.00 |
| Route | (mg) | Animal | Bumetanide Dibenzylamide (ng/mL) | | | | | | | | |
| PO | 30 | 2972702 | 0.00 | 0.00 | 7.82 | 110 | 158 | 68.7 | 21.9 | 10.5 | 2.84 |
| | | 2974674 | 0.00 | 0.850 | 43.7 | 122 | 175 | 56.9 | 15.7 | 7.91 | 2.83 |
| | | CJACUH | 0.00 | 3.99 | 36.8 | 129 | 259 | 95.8 | 21.5 | 10.3 | 2.31 |
| | | CJCCBU | 0.00 | 0.480 | 40.6 | 227 | 280 | 85.3 | 21.2 | 8.65 | 2.86 |
| | | Mean | 0.00 | 1.33 | 32.2 | 147 | 218 | 76.7 | 20.1 | 9.34 | 2.71 |
| | | CV % | | 135.9 | 51.2 | 36.7 | 27.7 | 22.5 | 14.6 | 13.5 | 9.9 |

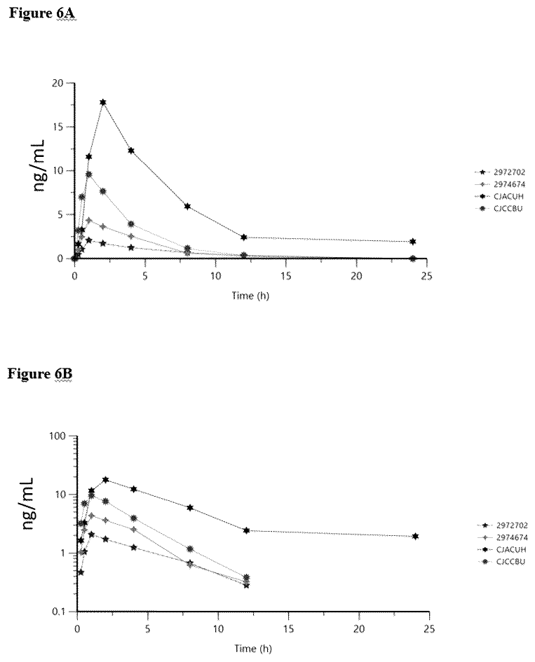

Plasma concentration data following IR administration of a 30 mg dose is illustrated in Table 8 and plotted in FIGS. 6A-6B. Mean concentrations of Bumetanide Dibenzylamide are less than 10 ng/mL at all times after dosing. Concentrations are variable through the 24-hour sampling period with % CV ranging from 64.4 to 200%. For the IR route of administration, each of the female dogs once again displayed higher concentrations than the 2 male dogs.

TABLE 7
Summary of Plasma Bumetanide Dibenzylamide Concentration-
Time Data in Dogs Following Administration of a Single
Dose of a Subcutaneous (SC) Formulation

| | | | Time (h) | | | | | | | | |
|---|---|---|---|---|---|---|---|---|---|---|---|
| | Dose | | 0.00 | 0.25 | 0.50 | 1.00 | 2.00 | 4.00 | 8.00 | 12.00 | 24.00 |
| Route | (mg) | Animal | Bumetanide Dibenzylamide (ng/mL) | | | | | | | | |
| SC | 15 | 2923344 | 1.55 | 1.78 | 1.81 | 2.24 | 3.18 | 4.15 | 4.14 | 3.22 | 2.90 |
| | | 2960045 | 1.39 | 6.40 | 2.08 | 15.2 | 4.74 | 6.61 | 6.67 | 5.09 | 3.70 |
| | | CJCCJW | 1.65 | 1.94 | 2.76 | 3.62 | 4.69 | 7.01 | 4.54 | 3.35 | 2.43 |
| | | CJDCDD | 2.39 | 2.86 | 3.42 | 4.96 | 7.90 | 11.3 | 7.47 | 5.77 | 4.30 |
| | | Mean | 1.75 | 3.25 | 2.52 | 6.51 | 5.13 | 7.27 | 5.71 | 4.36 | 3.33 |
| | | CV % | 25.4 | 66.5 | 28.7 | 90.7 | 38.7 | 40.9 | 28.4 | 29.2 | 24.9 |

TABLE 8
Summary of Plasma Bumetanide Dibenzylamide Concentration-Time Data in Dogs Following
Administration of a Single Dose of an Intrarectal (IR) Formulation

| | | | Time (h) | | | | | | | | |
|---|---|---|---|---|---|---|---|---|---|---|---|
| | Dose | | 0.00 | 0.25 | 0.50 | 1.00 | 2.00 | 4.00 | 8.00 | 12.00 | 24.00 |
| Route | (mg) | Animal | Bumetanide Dibenzylamide (ng/mL) | | | | | | | | |
| IR | 30 | 2972702 | 0.00 | 0.467 | 1.05 | 2.07 | 1.72 | 1.24 | 0.674 | 0.281 | 0.00 |
| | | 2974674 | 0.00 | 1.03 | 2.47 | 4.34 | 3.63 | 2.53 | 0.623 | 0.322 | 0.00 |
| | | CJACUH | 0.00 | 1.63 | 3.25 | 11.6 | 17.8 | 12.3 | 5.93 | 2.42 | 1.93 |
| | | CJCCBU | 0.00 | 3.18 | 6.99 | 9.56 | 7.64 | 3.92 | 1.17 | 0.383 | 0.00 |
| | | Mean | 0.00 | 1.58 | 3.44 | 6.89 | 7.70 | 5.00 | 2.10 | 0.852 | 0.483 |
| | | CV % | | 74.2 | 73.7 | 64.4 | 93.2 | 99.8 | 122.2 | 122.9 | 200.0 |

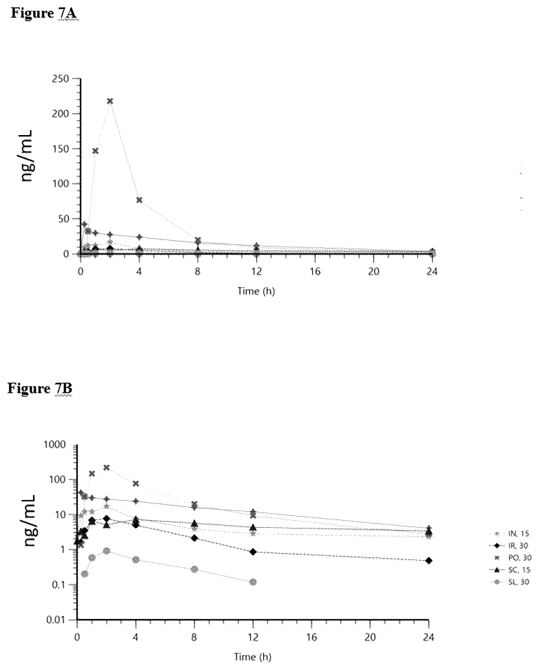

Mean concentration-time profiles for Bumetanide Dibenzylamide are plotted with formulations superimposed in FIGS. 7A-7B on linear and log-linear axes. Concentrations are substantially higher for PO administration than for any other route. The log-linear plots indicate that Bumetanide Dibenzylamide concentrations tend to decay in parallel after the 8-hour plasma sample.

Plasma Bumetanide concentration-time data are measured for all routes of administration. There are only 2 plasma samples in any sample from the study with measurable concentrations of Bumetanide. These measurements could be made in animal #7 at 2 and 4 hours after administration of the PO dose formulation and are 0.34 and 0.47 ng/ml, respectively (Table 9).

TABLE 9
Summary of Plasma Bumetanide Concentration-Time Data in Dogs Following
Administration of a Single Dose of an Oral (PO) Formulation

| | | | Time (h) | | | | | | | | |
|---|---|---|---|---|---|---|---|---|---|---|---|
| | Dose | | 0.00 | 0.25 | 0.50 | 1.00 | 2.00 | 4.00 | 8.00 | 12.00 | 24.00 |
| Route | (mg) | Animal | Bumetanide (ng/mL) | | | | | | | | |
| PO | 30 | 2972702 | 0.00 | 0.00 | 0.00 | 0.00 | 0.00 | 0.00 | 0.00 | 0.00 | 0.00 |
| | | 2974674 | 0.00 | 0.00 | 0.00 | 0.00 | 0.00 | 0.00 | 0.00 | 0.00 | 0.00 |
| | | CJACUH | 0.00 | 0.00 | 0.00 | 0.00 | 0.34 | 0.47 | 0.00 | 0.00 | 0.00 |
| | | CJCCBU | 0.00 | 0.00 | 0.00 | 0.00 | 0.00 | 0.00 | 0.00 | 0.00 | 0.00 |
| | | Mean | 0.000 | 0.000 | 0.000 | 0.000 | 0.086 | 0.119 | 0.000 | 0.000 | 0.000 |
| | | CV % | | | | | 200.0 | 200.0 | | | |

### Pharmacokinetic Data Analysis

In another embodiment described herein, pharmacokinetic (PK) parameters were derived using non-compartmental methods employing Phoenix WinNonlin® version 8.2 (Pharsight Corp; St. Louis, MO). The PK parameters defined in Table 10 are calculated by non-compartmental methods using individual animal concentration-time data. The area under the concentration-time curve from zero time (pre-dose) to the last time point is calculated by a combination of linear and logarithmic trapezoidal methods. The linear trapezoidal method is employed for all incremental trapezoids arising from increasing concentrations and the logarithmic trapezoidal method is used for those arising from decreasing concentrations (linear up/log down method). Nominal blood sampling times are used in the analysis. Concentrations of Bumetanide Dibenzylamide and Bumetanide below the lower limits of quantification (LLOQ) of 0.25 ng/mL are treated as zero for the computation of descriptive statistics for construction of mean concentration-time profiles. Individual below quantitation limits (BQL) results are set to zero for all calculations. No animals are excluded from the analysis. In one aspect, non-compartmental pharmacokinetic analysis provides observed or calculated values of Cₘₐₓ, Tmax, and AUClast for all animals and estimates of AUCinf and CL/F for those animals where a terminal phase half-life (t1/2) could be estimated.

TABLE 10
Pharmacokinetic Parameter Definitions

| | |
|---|---|
| Cmax | Maximum observed plasma concentration |
| Tmax | Time of maximum concentration, obtained directly from |
| | the observed concentration versus time data |
| C0 | Back predicted plasma concentration to time = 0 for IV |
| | bolus administration |
| AUCinf | The area under the concentration-time curve from time 0 |
| | extrapolated to infinity, calculated as AUClast + Clast/λz. |
| | Where >20% of AUCinf was derived by extrapolation, or |
| | where acceptance criteria for λz (defined below for t½) |
| | were not met, AUCinf was deemed not reportable. |
| CL | Clearance after IV dosing calculated as: |
| | Dose/AUCinf |
| Vz | Volume of distribution after IV dosing calculated as: |
| | Dose/(AUCinf)(λz) |
| t½ | Terminal phase half-life, estimated as ln(2)/λz, where λz is |
| | the elimination rate constant and was estimated by linear |
| | regression of logarithmically-transformed concentration |
| | versus time data (at least 3 measurable timepoints after |
| | Cmax). |
| Fabs | Absolute bioavailability calculated as: |
| | Fabs = (AUCpo/AUCiv) * (Dose iv/Dosepo) |

Non-compartmental pharmacokinetic parameters for Bumetanide Dibenzylamide after each route of administration are summarized in Tables 11-16. In one aspect, the 15 mg dose level data for IM, IN, and SC dosing suggests that IM provides the highest exposures with Cmax averaging 48.9 ng/mL and AUCinf of 411 h*ng/ml. In one aspect, SC dosing provides the next highest exposure with mean Cmax of 9.42 ng/ml and AUCinf of 231 h*ng/mL. IN dosing is comparable to SC displaying a higher Cmax of 22.0 ng/ml but slightly lower AUCinf of 211 h*ng/mL. In one aspect, the 30 mg dose level data for PO, IR, and SL dosing indicates that PO administration provides by far the highest exposures with Cmax averaging 218 ng/ml and a mean AUCinf of 814 h*ng/mL. In one aspect, IR dosing provides the next highest exposure with mean Cmax of 8.44 ng/ml and AUCinf of 55.1 h*ng/mL. In one aspect, SL dosing provides very low and inconsistent exposures with a mean Cmax of just 1.03 ng/ml and AUClast of only 4.23 h*ng/ml. Half-life could only be estimated in 1 of the 4 dogs following SL administration. As noted above there were only 2 measurable Bumetanide concentrations observed in the study and so no pharmacokinetic analysis was possible.

TABLE 11
Summary of Bumetanide Dibenzylamide Pharmacokinetic
Parameters in Dogs Following Administration of a
Single Dose of an Intranasal (IN) Formulation

| | Dose | | Cmax | Tmax | AUClast | t½ | AUCinf | CL/F |
|---|---|---|---|---|---|---|---|---|
| Route | (mg) | Animal | (ng/mL) | (h) | (h*ng/mL) | (h) | (h*ng/mL) | (L/h) |
| IN | 15 | 2923344 | 12.7 | 0.50 | 94.4 | 23.3 | 164 | 91.5 |
| | | 2960045 | 3.73 | 2.00 | 47.0 | 76.1 | 221 | 67.7 |
| | | CJCCJW | 41.9 | 2.00 | 165 | 19.8 | 234 | 64.1 |
| | | CJDCDD | 29.8 | 0.50 | 151 | 16.3 | 224 | 66.8 |
| | | Mean | 22.0 | 1.25 | 114 | 33.9 | 211 | 72.5 |
| | | CV % | 77.6 | 69.3 | 47.4 | 83.5 | 15.1 | 17.6 |

TABLE 12
Summary of Bumetanide Dibenzylamide Pharmacokinetic
Parameters in Dogs Following Administration of
a Single Dose of a Sublingual (SL) Formulation

| | Dose | | Cmax | Tmax | AUClast | t½ | AUCinf | CL/F |
|---|---|---|---|---|---|---|---|---|
| Route | (mg) | Animal | (ng/mL) | (h) | (h*ng/mL) | (h) | (h*ng/mL) | (L/h) |
| SL | 30 | 2972702 | 0.308 | 1.00 | 0.0770 | | | |
| | | 2974674 | 2.32 | 2.00 | 14.1 | 5.26 | 17.7 | 1690 |
| | | CJACUH | 1.00 | 2.00 | 2.17 | | | |
| | | CJCCBU | 0.505 | 1.00 | 0.547 | | | |
| | | Mean | 1.03 | 1.50 | 4.23 | 5.26 | 17.7 | 1690 |
| | | CV % | 87.7 | 38.5 | 157.4 | | | |

TABLE 13
Summary of Bumetanide Dibenzylamide Pharmacokinetic
Parameters in Dogs Following Administration of a
Single Dose of an Intramuscular (IM) Formulation

| | | | Cmax | Tmax | AUClast | t½ | AUCinf | CL/F |
|---|---|---|---|---|---|---|---|---|
| Route | Dose (mg) | Animal | (ng/mL) | (h) | (h*ng/mL) | (h) | (h*ng/mL) | (L/h) |
| IM | 15 | 2923344 | 13.1 | 2.00 | 135 | 18.7 | 215 | 69.6 |
| | | 2960045 | 155 | 0.25 | 926 | 6.16 | 999 | 15.0 |
| | | CJCCJW | 4.87 | 4.00 | 63.0 | 38.1 | 166 | 90.5 |
| | | CJDCDD | 22.7 | 2.00 | 202 | 13.4 | 268 | 56.0 |
| | | Mean | 48.9 | 2.06 | 331 | 19.1 | 412 | 57.8 |
| | | CV % | 145.3 | 74.3 | 120.8 | 71.6 | 95.5 | 55.1 |

TABLE 14
Summary of Bumetanide Dibenzylamide Pharmacokinetic Parameters in Dogs
Following Administration of a Single Dose of an Oral (PO) Formulation

| | | | Cmax | Tmax | AUClast | t½ | AUCinf | CL/F |
|---|---|---|---|---|---|---|---|---|
| Route | Dose (mg) | Animal | (ng/mL) | (h) | (h*ng/mL) | (h) | (h*ng/mL) | (L/h) |
| PO | 30 | 2972702 | 158 | 2.00 | 675 | 5.62 | 698 | 43.0 |
| | | 2974674 | 175 | 2.00 | 639 | 6.79 | 666 | 45.0 |
| | | CJACUH | 259 | 2.00 | 893 | 5.10 | 910 | 33.0 |
| | | CJCCBU | 280 | 2.00 | 956 | 5.89 | 980 | 30.6 |
| | | Mean | 218 | 2.00 | 791 | 5.85 | 814 | 37.9 |
| | | CV % | 27.7 | 0.0 | 19.9 | 12.1 | 19.1 | 18.9 |

TABLE 15
Summary of Bumetanide Dibenzylamide Pharmacokinetic Parameters in Dogs
Following Administration of a Single Dose of a Subcutaneous (SC) Formulation

| | | | Cmax | Tmax | AUClast | t½ | AUCinf | CL/F |
|---|---|---|---|---|---|---|---|---|
| Route | Dose (mg) | Animal | (ng/mL) | (h) | (h*ng/mL) | (h) | (h*ng/mL) | (L/h) |
| SC | 15 | 2923344 | 4.15 | 4.00 | 79.8 | 36.2 | 231 | 64.8 |
| | | 2960045 | 15.2 | 1.00 | 129 | 22.2 | 247 | 60.7 |
| | | CJCCJW | 7.01 | 4.00 | 91.3 | 19.1 | 158 | 94.7 |
| | | CJDCDD | 11.3 | 4.00 | 153 | 21.5 | 286 | 52.4 |
| | | Mean | 9.42 | 3.25 | 113 | 24.8 | 231 | 68.2 |
| | | CV % | 51.5 | 46.2 | 29.7 | 31.3 | 23.2 | 27.0 |

TABLE 16
Summary of Bumetanide Dibenzylamide Pharmacokinetic Parameters in Dogs
Following Administration of a Single Dose of an Intranasal (IN) Formulation

| | | | Cmax | Tmax | AUClast | t½ | AUCinf | CL/F |
|---|---|---|---|---|---|---|---|---|
| Route | Dose (mg) | Animal | (ng/mL) | (h) | (h*ng/mL) | (h) | (h*ng/mL) | (L/h) |
| IN | 15 | 2923344 | 12.7 | 0.50 | 94.4 | 23.3 | 164 | 91.5 |
| | | 2960045 | 3.73 | 2.00 | 47.0 | 76.1 | 221 | 67.7 |
| | | CJCCJW | 41.9 | 2.00 | 165 | 19.8 | 234 | 64.1 |
| | | CJDCDD | 29.8 | 0.50 | 151 | 16.3 | 224 | 66.8 |
| | | Mean | 22.0 | 1.25 | 114 | 33.9 | 211 | 72.5 |
| | | CV % | 77.6 | 69.3 | 47.4 | 83.5 | 15.1 | 17.6 |

In one embodiment, the pharmacokinetic profile of Bumetanide Dibenzylamide, when administered as a single oral gavage or as an intravenous dose to rats was evaluated.

### Study Design

Male Hsd: Sprague Dawley rats were assigned to two groups, and doses were administered as indicated in Table 17. Animals were dosed via oral gavage or intravenous injection once on Day 1 at a volume of 5 mL/kg for Group 1 and 10 mL/kg for Group 2. The vehicle was 40% (v/v) Polyethylene Glycol 400 (PEG400), 20% (v/v) Propylene Glycol, 5% (v/v) Ethanol, and 35% (v/v) water for Group 1 and the vehicle was 0.5% (w/v) Carboxymethylcellulose (medium viscosity) in reverse osmosis water for Group 2.

TABLE 17
Group Assignments and Treatment Information for Phase I

| | | No. of | | | Volume | Dose |
|---|---|---|---|---|---|---|
| | | Animals^{a, b} | | Dose Level | Dose | Concentration |
| Group | Subgroup | Males | Dose Route | (mg/kg) | (mL/kg) | (mg/mL) |
| 1 | 1 | 6 | Intravenous | 10 | 5 | 2 |
| | (Toxicokinetic) | | | | | |
| 2 | 1 | 6 | Oral Gavage | 30 | 10 | 3 |
| | (Toxicokinetic) | | | | | |

^{a}All animals were dosed on Day 1 of the dosing phase.
^{b}Phase I animals were used for the Pharmacokinetic profile only.

Oral administration was selected because it is the intended route of administration in humans. The dose levels chosen for Phase I were based on tolerability data following intravenous (IV) administration in dogs, allometrically scaled to the rat.

### Plasma Concentration Data

Blood samples (approximately 0.5 mL) were collected from nonfasted toxicokinetic animals via a jugular vein on Day 1 of the dosing phase, as indicated in Table 18

TABLE 18
Blood Collection

| | | Dosing | |
|---|---|---|---|
| Group | Set | Phase Day | Time Pointsª |
| 1 | 1st three | 1 | 5 minutes; 2, 6, and |
| | animals/ | | 12 hours postdose |
| | sex/group | | |
| 1 | 2nd three | 1 | 1, 4, 8, and 24 hours |
| | animals/ | | postdose |
| | sex/group | | |
| 2 | 1st three | 1 | 30 minutes; 2, 6, and |
| | animals/ | | 12 hours postdose |
| | sex/group | | |
| 2 | 2nd three | 1 | 1, 4, 8, and 24 hours |
| | animals/ | | postdose |
| | sex/group | | |

^{a}Blood collection times were approximate.

Blood was collected into tubes containing potassium (K2) EDTA as the anticoagulant. Samples were maintained on wet ice and were centrifuged within 30 minutes of collection. Plasma was harvested and stored on dry ice until placed in a freezer, set to maintain −60 to −80° C., until shipped on dry ice to Origin Bioanalytical Laboratory, Inc. for analysis. Plasma samples were analyzed for Bumetanide Dibenzylamide and Bumetanide content.

### Pharmacokinetic Data Analysis

Pharmacokinetic parameters were derived using non-compartmental methods employing Phoenix WinNonlin® version 8.1 (Pharsight Corp; St. Louis, MO). The PK parameters defined previously in Table 10 were calculated by non-compartmental methods using composite plasma concentration-time data obtained from 3 rats/sex/time point/dose. The area under the concentration-time curve from zero time (pre-dose) to the last time point was calculated by a combination of linear and logarithmic trapezoidal methods. The linear trapezoidal method was employed for all incremental trapezoids arising from increasing concentrations and the logarithmic trapezoidal method was used for those arising from decreasing concentrations (linear up/log down method). Nominal blood sampling times were used in the analysis. Concentrations of Bumetanide Dibenzylamide below the lower limit of quantification (LLOQ) of 0.25 ng/ml were treated as zero for the computation of descriptive statistics for construction of mean concentration-time profiles. Individual BQL results were set to zero for all calculations. No animals were excluded from the analysis.

### Plasma Bumetanide Dibenzylamide and Bumetanide Concentration-Time Data

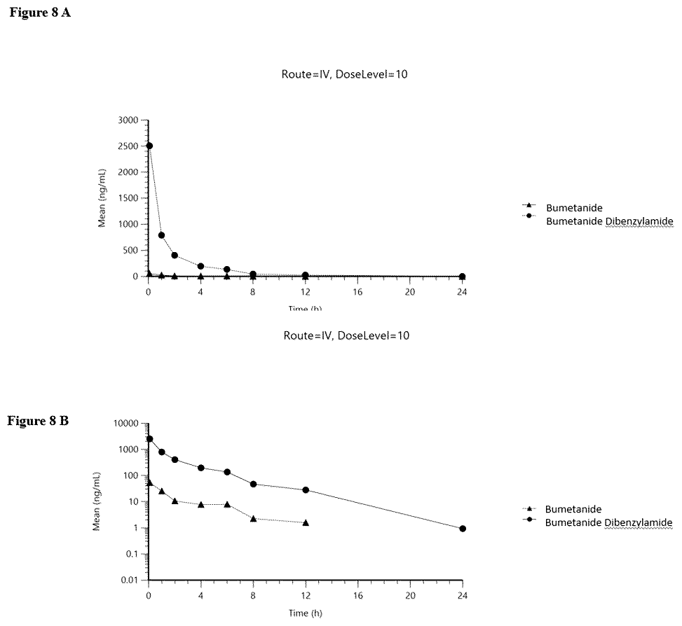

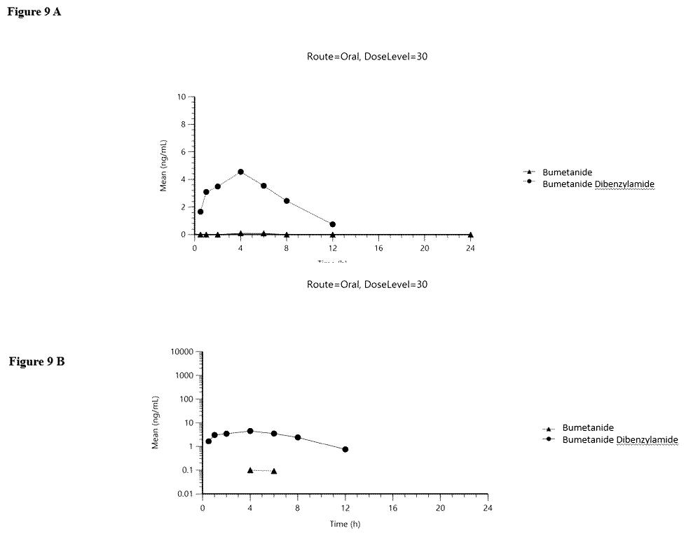

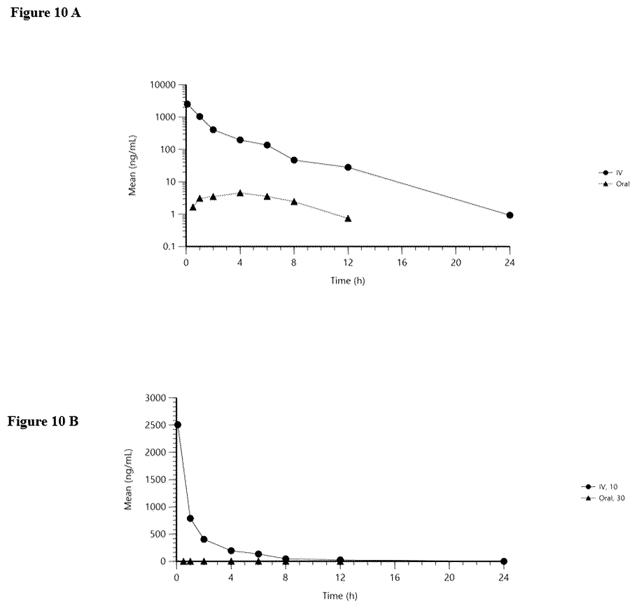

Plasma Bumetanided Dibenzylamide and Bumetanide concentration time data are summarized following IV administration of 10 mg/kg Bumetanide Dibenzylamide in Tables 19-20, respectively. Mean concentration-time profiles are illustrated with analytes overlaid in FIGS. 8A-8B on linear and log-linear axes. Observed Bumetanide Dibenzylamide concentration averaged 2510 ng/ml at 5 minutes after the IV bolus dose. Measurable concentrations in all 3 animals were observed at 24 hours after dosing. In contrast, Bumetanide concentrations were lower, averaging 53.6 ng/mL at 5 minutes and no animals had measurable concentrations at 24 hours after dosing. Plasma concentration time data was summarized following oral administration of 30 mg/kg Bumetanide Dibenzylamide in Error! Reference source not found. 21 and mean concentration-time profiles with analytes overlaid are displayed in Error! Reference source not found. The highest mean concentration of Bumetanide Dibenzylamide was 4.56 ng/ml occurring 4 hours after dosing. Bumetanide concentrations were negligible with only 2 rats having measurable concentrations. One was 0.30 ng/mL at 4 hours and the other was 0.28 ng/mL at the 6-hour sampling time.

Mean plasma Bumetanide Dibenzylamide concentrations plotted in Error! Reference source not found. reveal that Bumetanide Dibenzylamide concentrations are considerably lower after oral versus IV bolus dosing. The individual rat plasma concentration-time data used in the analysis are presented in Table 22.

TABLE 19
Summary of Plasma Bumetanide Dibenzylamide Concentrations in Male Rats
Following IV Bolus Administration of 10 mg/kg Bumetanide Dibenzylamide

| | | | Time (h) | | | | | | | |
|---|---|---|---|---|---|---|---|---|---|---|
| | Dose | | 0.08 | 1.00 | 2.00 | 4.00 | 6.00 | 8.00 | 12.00 | 24.00 |
| Route | (mg/kg) | Animal | Bumetanide Dibenzylamide (ng/mL) | | | | | | | |
| IV | 10 | R0001 | 1720 | | 317 | | 203 | | 60.8 | |
| | | R0002 | 3080 | | 477 | | 145 | | 18.6 | |
| | | R0003 | 2720 | | 421 | | 60.8 | | 4.67 | |
| | | R0004 | | 993 | | 165 | | 20.0 | | 0.257 |
| | | R0005 | | 1190 | | 205 | | 33.1 | | 0.582 |
| | | R0006 | | 924 | | 218 | | 87.3 | | 1.95 |
| | | N | 3 | 3 | 3 | 3 | 3 | 3 | 3 | 3 |
| | | Mean | 2510 | 1040 | 405 | 196 | 136 | 46.8 | 28.0 | 0.930 |
| | | CV % | 28.1 | 13.3 | 20.0 | 14.1 | 52.5 | 76.2 | 104.3 | 96.6 |

TABLE 20
Summary of Plasma Bumetanide Concentrations in Male Rats Following
IV Bolus Administration of 10 mg/kg Bumetanide Dibenzylamide

| | | | Time (h) | | | | | | | |
|---|---|---|---|---|---|---|---|---|---|---|
| | Dose | | 0.08 | 1.00 | 2.00 | 4.00 | 6.00 | 8.00 | 12.00 | 24.00 |
| Route | (mg/kg) | Animal | Bumetanide (ng/mL) | | | | | | | |
| IV | 10 | R0001 | 44.5 | | 8.33 | | 9.24 | | 3.48 | |
| | | R0002 | 60.3 | | 13.2 | | 11.6 | | 1.29 | |
| | | R0003 | 55.9 | | 10.5 | | 3.09 | | 0.00 | |
| | | R0004 | | 26.9 | | 7.36 | | 0.967 | | 0.00 |
| | | R0005 | | 24.9 | | 7.07 | | 1.35 | | 0.00 |
| | | R0006 | | 25.2 | | 9.01 | | 4.46 | | 0.00 |
| | | N | 3 | 3 | 3 | 3 | 3 | 3 | 3 | 3 |
| | | Mean | 53.6 | 25.7 | 10.7 | 7.81 | 7.98 | 2.26 | 1.59 | 0.00 |
| | | CV % | 15.2 | 4.2 | 22.9 | 13.4 | 55.1 | 84.8 | 110.6 | |

TABLE 21
Summary of Plasma Bumetanide Dibenzylamide Concentrations in Male Rats
Following Oral Administration of 30 mg/kg Bumetanide Dibenzylamide

| | | | Time (h) | | | | | | | |
|---|---|---|---|---|---|---|---|---|---|---|
| | Dose | | 0.50 | 1.00 | 2.00 | 4.00 | 6.00 | 8.00 | 12.00 | 24.00 |
| Route | (mg/kg) | Animal | Bumetanide Dibenzylamide (ng/mL) | | | | | | | |
| Oral | 30 | R0101 | 2.01 | | 4.00 | | 3.99 | | 0.780 | |
| | | R0102 | 1.29 | | 2.88 | | 2.49 | | 0.715 | |
| | | R0103 | 1.67 | | 3.60 | | 4.15 | | 0.731 | |
| | | R0104 | | 2.14 | | 3.52 | | 1.57 | | |
| | | R0105 | | 4.70 | | 5.42 | | 2.57 | | |
| | | R0106 | | 2.45 | | 4.73 | | 3.20 | | |
| | | N | 3 | 3 | 3 | 3 | 3 | 3 | 3 | 0 |
| | | Mean | 1.66 | 3.10 | 3.49 | 4.56 | 3.54 | 2.45 | 0.742 | |
| | | CV % | 21.7 | 45.1 | 16.2 | 21.1 | 25.8 | 33.6 | 4.6 | |

TABLE 22
Summary of Plasma Bumetanide Concentrations in Male Rats
Following
Oral Administration of 30 mg/kg Bumetanide Dibenzylamide

| | | | Time (h) | | | | | | | |
|---|---|---|---|---|---|---|---|---|---|---|
| | Dose | | 0.50 | 1.00 | 2.00 | 4.00 | 6.00 | 8.00 | 12.00 | 24.00 |
| Route | (mg/kg) | Animal | Bumetanide (ng/mL) | | | | | | | |
| Oral | 30 | R0101 | 0.00 | | 0.00 | | 0.00 | | 0.00 | |
| | | R0102 | 0.00 | | 0.00 | | 0.00 | | 0.00 | |
| | | R0103 | 0.00 | | 0.00 | | 0.28 | | 0.00 | |
| | | R0104 | | 0.00 | | 0.00 | | 0.00 | | 0.00 |
| | | R0105 | | 0.00 | | 0.30 | | 0.00 | | 0.00 |
| | | R0106 | | 0.00 | | 0.00 | | 0.00 | | 0.00 |
| | | N | 3 | 3 | 3 | 3 | 3 | 3 | 3 | 3 |
| | | Mean | 0.00 | 0.00 | 0.00 | 0.100 | 0.0920 | 0.00 | 0.00 | 0.00 |
| | | CV % | | | | 173.2 | 173.2 | | | |

### Pharmacokinetic Parameters for Bumetanide Dibenzylamide and Bumetanide

Non-compartmental PK parameters for Bumetanide Dibenzylamide and Bumetanide after IV dosing are summarized in Tables 23-24. The back-predicted concentration of Bumetanide Dibenzylamide at time-0 was 2780 ng/ml and the terminal phase half-life was 2.61 hours. CL was high at 2880 mL/h/kg and the Vz was large at 10800 L/kg. Bumetanide concentrations were considerably lower with a Cₘₐₓ of 53.6 ng/ml occurring at 5 minutes. The half-life was similar to that of Bumetanide Dibenzylamide at 2.91 hours. Non-compartmental PK parameters for Bumetanide Dibenzylamide after oral dosing are summarized in Table 25. The maximum concentration was low at 4.56 ng/mL occurring at 4 hours after dosing. The half-life was identical to the value obtained following IV dosing, at 2.61 hours.

TABLE 23
Pharmacokinetic Parameters for Bumetanide Dibenzylamide in
Rats following an IV bolus dose of 10 mg/kg Bumetanide
Dibenzylamide

| Dose | | | | | | CL | Vz |
|---|---|---|---|---|---|---|---|
| Level | Cmax | Tmax | C0 | t½ | AUCinf | (mL/ | (mL/ |
| (mg/kg) | (ng/ml) | (h) | (ng/ml) | (h) | (h*ng/ml) | h/kg) | kg) |
| 10 | 2510 | 0.08 | 2720 | 2.61 | 3730 | 2680 | 10100 |

TABLE 24
Pharmacokinetic Parameters for Bumetanide in Rats following an
IV bolus dose of 10 mg/kg Bumetanide Dibenzylamide

| | | Dose | Cmax | Tmax | t½ | AUCinf |
|---|---|---|---|---|---|---|
| Variable | Route | (mg/kg) | (ng/mL) | (h) | (h) | (h * ng/mL) |
| Bumetanide | IV | 10 | 53.6 | 0.08 | 2.91 | 112 |

TABLE 25
Pharmacokinetic Parameters for Bumetanide Dibenzylamide in Rats
following an Oral dose of 30 mg/kg Bumetanide Dibenzylamide

| | | Dose | Cmax | Tmax | t½ | AUCinf |
|---|---|---|---|---|---|---|
| Variable | Route | (mg/kg) | (ng/mL) | (h) | (h) | (h * ng/mL) |
| Bumetanide | Oral | 30 | 4.56 | 4.00 | 2.61 | 35.4 |
| Dibenzylamide | | | | | | |

### Absolute Bioavailability (Fabs) of Bumetanide Dibenzylamide Following Oral and IV Administration to Male Rats

Absolute bioavailability calculations for Bumetanide Dibenzylamide are summarized in Table 26. The AUCinf for Bumetanide Dibenzylamide following IV bolus dosing at a dose level of 10 mg/kg was 3470 h*ng/ml and the AUCinf following oral dosing of 30 mg/kg was 35.4 h*ng/mL. Adjusting for the doses administered the fraction of Bumetanide Dibenzylamide absorbed was 0.0034 or 0.34%.

TABLE 26
Absolute Bioavailability of Bumetanide Dibenzylamide in Rats

| | | Dose | AUCinf | Fabs | Fabs |
|---|---|---|---|---|---|
| Variable | Route | (mg/kg) | (h * ng/ml) | (ratio) | (%) |
| Bumetanide | Oral | 30 | 35.4 | 0.0032 | 0.32% |
| Dibenzylamide | IV | 10 | 3730 | | |

Though measurable concentrations of Bumetanide Dibenzylamide were observed in rats after IV and oral doses, CL and Vz values after IV administration (2680 mL/h/kg and 10100 mL/kg, respectively) indicate that Bumetanide Dibenzylamide has a high clearance and large volume of distribution in rats. The absolute bioavailability was determined to be very low in the rat, with a fraction absorbed of 0.0032 (0.32%), suggesting the possibility of very high first-pass extraction by the liver. Bumetanide Dibenzylamide Cₘₐₓ averaged 2510 ng/mL at 5 minutes after administration of the IV dose and 4.56 ng/ml at a Tmax of 4 hours following oral dosing. Measurable concentrations of Bumetanide were observed following IV dosing of Bumetanide Dibenzylamide with an average Cₘₐₓ of 53.6 ng/ml at 5 minutes after dose administration. The Bumetanide concentrations were below the lower limit of quantification of the bioanalytical method (0.25 ng/mL) in all but 2 samples following oral administration of 30 mg/kg Bumetanide Dibenzylamide. The terminal phase half-lives for Bumetanide Dibenzylamide were identical after each route of administration at 2.61 hours. The half-life of Bumetanide after IV administration of Bumetanide Dibenzylamide was 2.91 hours. CL and Vz values after IV administration were 2680 mL/h/kg and 10100 mL/kg indicating that Bumetanide Dibenzylamide has a high clearance and large volume of distribution in rats.

Oral Delivery of Unformulated Bumetanide Dibenzylamide In one embodiment, the pharmacokinetic profile and bioavailability of Bumetanide Dibenzylamide, and the formation of bumetanide as a metabolite of Bumetanide Dibenzylamide, following oral dose administration of non-formulated Bumetanide Dibenzylamide was evaluated in Beagle dogs.

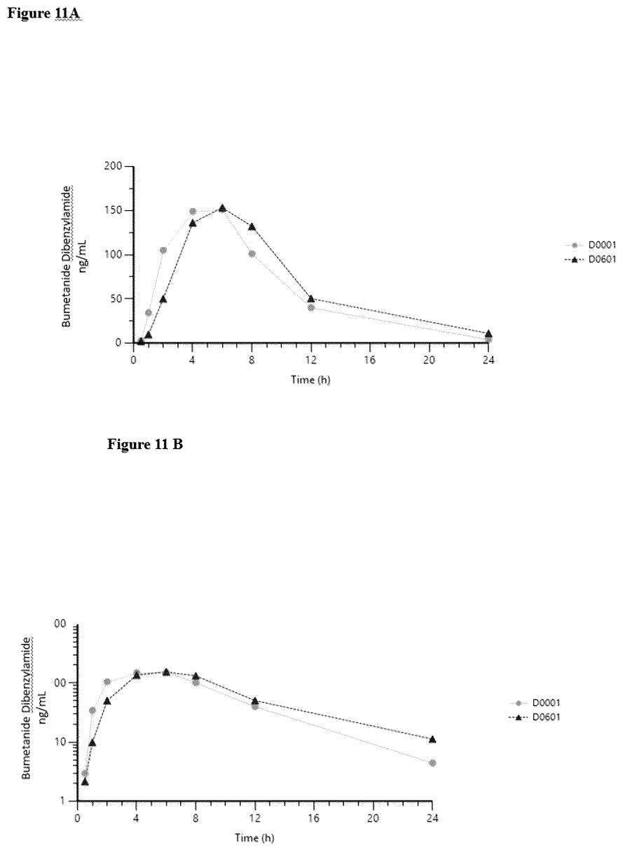

The pharmacokinetic profiles and bioavailability of Bumetanide Dibenzylamide, and the formation of Bumetanide as a metabolite of Bumetanide Dibenzylamide, following oral dose administration was evaluated in in Beagle dogs. The dose level used was about 30 mg of Bumetanide Dibenzylamide. 2 dogs were given the dosage and the Bumetanide Dibenzylamide levels (Table 27) were recorded as illustrated in FIGS. 11A-11B. Bumetanide levels were not measureable.

TABLE 27
Summary of Plasma Bumetanide Dibenzylamide Concentrations
in Dogs Following Oral Administration of Non-Formulated
30 mg/kg Bumetanide Dibenzylamide

| Dose | | Time | | | | | | | |
|---|---|---|---|---|---|---|---|---|---|
| Level | | (h) | | | | | | | |
| (mg/ | | 0.50 | 1.00 | 2.00 | 4.00 | 6.00 | 8.00 | 12.00 | 24.00 |
| kg) | Animal | Bumetanide Dibenzylamide (ng/mL) | | | | | | | |
| 30 | D0001 | 2.99 | 34.5 | 105 | 149 | 151 | 101 | 40.1 | 4.48 |
| | D0601 | 2.17 | 9.94 | 50.3 | 136 | 153 | 132 | 50.5 | 11.3 |
| | Mean | 2.58 | 22.2 | 77.7 | 143 | 152 | 117 | 45.3 | 7.89 |
| | Min | 2.17 | 9.94 | 50.3 | 136 | 151 | 101 | 40.1 | 4.48 |
| | Max | 2.99 | 34.5 | 105 | 149 | 153 | 132 | 50.5 | 11.3 |

Absolute bioavailability calculations for Bumetanide Dibenzylamide are summarized in Table 28. Absolute Bioavailability was determined to be about 5.1%

TABLE 28
Absolute Bioavailability of Bumetanide Dibenzylamide in Dogs

| | Dose | | | | | | | | |
|---|---|---|---|---|---|---|---|---|---|
| | Level | | Cmax | Tmax | AUClast | AUCinf | t½ | CL/F | Vz/F |
| Route | (mg/kg) | Animal | (ng/mL) | (h) | (h * ng/mL) | (h * ng/mL) | (h) | (L/h/kg) | (L/kg) |
| Oral | 30 | D0001 | 151 | 6.00 | 1340 | 1360 | 3.61 | 22.0 | 115 |
| Oral | 30 | D0601 | 153 | 6.00 | 1450 | 1520 | 4.72 | 19.7 | 134 |
| | | Mean | 152 | 6.00 | 1390 | 1440 | 4.16 | 20.8 | 124 |
| | | Min | 151 | 6.00 | 1340 | 1360 | 3.61 | 19.7 | 115 |
| | | Max | 153 | 6.00 | 1450 | 1520 | 4.72 | 22.0 | 134 |

It will be apparent to one of ordinary skill in the relevant art that suitable modifications and adaptations to the compositions, formulations, methods, processes, reactions, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any and all variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof. In one embodiment, the the concentrations of Bumetanide Dibenzylamide and Bumetanide in monkey plasma (K2EDTA) by LC-MS/MS was analyzed using a qualified bioanalytical method.

### Study Design

A pharmacokinetic study was done in cynomolgus monkeys following single oral (nasogastric) gavage, sublingual, and intravenous administration. Briefly, Bumetanide Dibenzylamide and Bumetanide and the respective internal standards, Bumetanide Dibenzylamide-d5 and Bumetanide-d5, were extracted from 100 μL of monkey plasma using protein precipitation extraction. Calibration curves, blanks, and analytical QCs were prepared in monkey plasma (K2EDTA). The extracts were evaporated to dryness, reconstituted, and then analyzed by LC-MS/MS. The calibration range was 0.250-250 ng/mL for both analytes. Analytical runs in this study were evaluated for acceptance based on the criteria listed in Table 29.

TABLE 29
Acceptance Criteria for Calibration Curves

| Item | Acceptance Criteria |
|---|---|
| Calibration | Accuracy within ± 15% (±20% at the LLOQ) |
| Curve: | ≥75% of standards within accuracy criteria |
| Analytical | Accuracy within ± 20% |
| QCs: | ≥2/3 of QCs within accuracy criteria |
| | ≥50% of QCs within accuracy criteria at each level |
| Dilution | Accuracy within ± 20% |
| QCs: | ≥2/3 of QCs within accuracy criteria for each |
| | dilution scheme |

If the criteria for either the calibration curve or the analytical QCs were not met, the analytical run was rejected. If the dilution QCs for a given dilution scheme did not meet criteria, then all of the study samples associated with that dilution scheme were rejected. No deviations were found during the bioanalytical phase of the study conducted by Origin. Calibration curves were derived from the individual analyte to internal standard peak area ratios using the least squared regression of the ratio versus the nominal concentration of the calibration standard, prepared in duplicate. The regression was then used to back-calculate the concentration of each calibration standard, QC sample and study sample. Quadratic (1/x²) regression was used for Bumetanide Dibenzylamide and Linear (1/x²) regression was used for Bumetanide. Analyst® Software Version 1.6.3 (Applied Biosystems-MDS Sciex), operated with Windows® (Microsoft), was used for instrument control, data acquisition, and peak integrations. Peak area ratios, standard curve regressions, and sample concentration/accuracy values were calculated with Analyst® Software Version 1.6.3. Other results (e.g., mean statistics) were calculated with Excel® (Microsoft).

### Pharmacokinetic and Bioavailability Data

Noncompartmental pharmacokinetic parameters for Bumetanide Dibenzylamide at a 30 mg/kg dose level data for PO administration of a first formulation comprising about 3% Bumetanide Dibenzylamide, Caprylocaproyl Polyoxylglycerides (Labrasol ALF), Propylene Glycol, Polyethylene Glycol and water suggested good bioavailability with both males exhibiting higher concentrations than the females. The Cmax and AUCinf values for the 30 mg/kg dose for NG (Single Oral Nasogastric Gavage) administration of a second formulation comprising about 1.73 w/w % Bumetanide Dibenzylamide, about 32.4 w/w % Polyoxyl 35 Castor Oil (Kolliphor EL), about 31.32 w/w %), Glyceryl Monolinoleate (Maisine CC), about 31.32 w/w % Soybean Oil, and about 3.24 w/w % Ethanol, which is SEDDS/oral lymphatic targeted formulation, were much higher, with an 8-fold higher Cmax and a 6-fold higher AUCinf. The concentration-time profiles were different from those observed for the first formulation since Bumetanide Dibenzylamide displayed a later median Tmax and a shorter half-life. There was no apparent sex difference for the second formulation. The pharmacokinetic parameters for Bumetanide Dibenzylamide following an IV bolus a third formulation-comprising about 0.2% Bumetanide Dibenzylamide, Ethanol, Propylene Glycol, 40% Polyethylene Glycol 400, water, administered at a dose level of 2 mg/kg, were used to provide estimates of the absolute bioavailability of Bumetanide Dibenzylamide after PO dosing and values of the true CL and Vz values in monkeys. The absolute bioavailability (% Fabs) calculations for the PO formulations based on Cₘₐₓ and AUCinf indicated that the second formulation/SEDDS displayed a 6-fold increase in % Fabs than the first formulation (from 2.5% to 15%). There were only a limited number of measurable plasma samples containing bumetanide following PO or IV administration. There were no measurable concentrations of Bumetanide after SL dosing. A fourth formulation comprising the composition B listed in Table 54 was also administered in monheys. Tables 30-33 summarize plasma Bumetanide Dibenzylamide concentrations in monkeys following administration of the various formulations.

TABLE 30
Plasma Bumetanide Dibenzylamide Concentrations in Monkey
Following Single Oral (Nasogastric) Gavage of Non-Formulated 30 mg/kg Bumetanide
Dibenzylamide (First Formulation).

| | | | | | Time | | | | | | | | |
|---|---|---|---|---|---|---|---|---|---|---|---|---|---|
| | | | | | (h) | | | | | | | | |
| | | | | | 0.00 | 0.25 | 0.50 | 1.00 | 2.00 | 4.00 | 8.00 | 24.00 | 32.00 |
| | | Dose | | | Bumetanide Dibenzylamide | | | | | | | | |
| Form | Route | (mg/kg) | Animal | Sex | (ng/mL) | | | | | | | | |
| 1 | NG | 30 | 23153 | M | 0.00 | 0.932 | 3.97 | 12.1 | 157 | 182 | 99.9 | 21.6 | 9.75 |
| | | | 23154 | M | 0.00 | 14.9 | 16.8 | 29.7 | 74.0 | 51.4 | 45.1 | 7.47 | 6.13 |
| | | | 23155 | F | 0.00 | 0.992 | 1.66 | 8.08 | 11.1 | 9.83 | 17.5 | 3.28 | 0.790 |
| | | | 23156 | F | 0.00 | 0.398 | 1.18 | 3.01 | 8.35 | 23.8 | 17.0 | 2.02 | 1.37 |
| | | | | N | 4 | 4 | 4 | 4 | 4 | 4 | 4 | 4 | 4 |
| | | | | Mean | 0.00 | 4.31 | 5.90 | 13.2 | 62.6 | 66.8 | 44.9 | 8.59 | 4.51 |
| | | | | SD | 0.00 | 7.07 | 7.37 | 11.6 | 69.8 | 78.7 | 39.0 | 8.98 | 4.23 |
| | | | | CV % | | 164.2 | 124.8 | 87.7 | 111.6 | 118.0 | 86.8 | 104.5 | 93.9 |
| | | | | Min | 0.00 | 0.398 | 1.18 | 3.01 | 8.35 | 9.83 | 17.0 | 2.02 | 0.790 |
| | | | | Median | 0.00 | 0.962 | 2.82 | 10.1 | 42.6 | 37.6 | 31.3 | 5.38 | 3.75 |
| | | | | Max | 0.00 | 14.9 | 16.8 | 29.7 | 157 | 182 | 99.9 | 21.6 | 9.75 |

TABLE 31
Plasma Bumetanide Dibenzylamide Concentrations in Monkey
Following Single Oral (Nasogastric) Gavage of Non-Formulated 30 mg/kg Bumetanide
Dibenzylamide (Second Formulation).

| | | | | | Time | | | | | | | | |
|---|---|---|---|---|---|---|---|---|---|---|---|---|---|
| | | | | | (h) | | | | | | | | |
| | | | | | 0.00 | 0.25 | 0.50 | 1.00 | 2.00 | 4.00 | 8.00 | 24.00 | 32.00 |
| | | Dose | | | Bumetanide Dibenzylamide | | | | | | | | |
| Form | Route | (mg/kg) | Animal | Sex | (ng/mL) | | | | | | | | |
| 3 | NG | 30 | 23153 | M | 0.00 | 0.250 | 1.55 | 7.56 | 16.8 | 126 | 442 | 26.4 | 6.41 |
| | | | 23154 | M | 0.00 | 0.00 | 3.16 | 11.3 | 82.5 | 410 | 785 | 8.13 | 2.08 |
| | | | 23155 | F | 0.00 | 0.619 | 4.98 | 20.2 | 79.3 | 140 | 457 | 9.30 | 1.63 |
| | | | 23156 | F | 0.00 | 0.261 | 1.71 | 7.09 | 37.8 | 247 | 674 | 10.7 | 1.62 |
| | | | | N | 4 | 4 | 4 | 4 | 4 | 4 | 4 | 4 | 4 |
| | | | | Mean | 0.00 | 0.283 | 2.85 | 11.5 | 54.1 | 231 | 590 | 13.6 | 2.94 |
| | | | | SD | 0.00 | 0.255 | 1.59 | 6.07 | 32.1 | 131 | 168 | 8.58 | 2.33 |
| | | | | CV % | | 90.1 | 55.9 | 52.6 | 59.4 | 56.8 | 28.5 | 62.9 | 79.3 |
| | | | | Min | 0.00 | 0.00 | 1.55 | 7.09 | 16.8 | 126 | 442 | 8.13 | 1.62 |
| | | | | Median | 0.00 | 0.256 | 2.44 | 9.43 | 58.6 | 194 | 566 | 10.0 | 1.86 |
| | | | | Max | 0.00 | 0.619 | 4.98 | 20.2 | 82.5 | 410 | 785 | 26.4 | 6.41 |

TABLE 32
Plasma Bumetanide Dibenzylamide Concentrations in Monkey
Following Intravenous Administration of 30 mg/kg Bumetanide Dibenzylamide (Third
Formulation).

| | | | | | Time | | | | | | | |
|---|---|---|---|---|---|---|---|---|---|---|---|---|
| | | | | | (h) | | | | | | | |
| | | | | | 0.08 | 0.50 | 1.00 | 2.00 | 4.00 | 8.00 | 24.00 | 32.00 |
| | | Dose | | | Bumetanide Dibenzylamide | | | | | | | |
| Form | Route | (mg/kg) | Animal | Sex | (ng/mL) | | | | | | | |
| 4 | IV | 2 | 23153 | M | 1860 | 717 | 538 | 332 | 149 | 51.4 | 3.95 | 1.47 |
| | | | 23154 | M | 1430 | 757 | 536 | 316 | 87.8 | 26.6 | 0.942 | 0.303 |
| | | | 23155 | F | 1160 | 555 | 472 | 278 | 129 | 27.7 | 8.10 | 3.80 |
| | | | 23156 | F | 1310 | 626 | 385 | 214 | 89.3 | 17.2 | 1.28 | 0.363 |
| | | | | N | 4 | 4 | 4 | 4 | 4 | 4 | 4 | 4 |
| | | | | Mean | 1440 | 664 | 483 | 285 | 114 | 30.7 | 3.57 | 1.48 |
| | | | | SD | 301 | 90.9 | 72.0 | 52.5 | 30.3 | 14.6 | 3.31 | 1.63 |
| | | | | CV % | 20.9 | 13.7 | 14.9 | 18.4 | 26.6 | 47.4 | 92.7 | 110.1 |
| | | | | Min | 1160 | 555 | 385 | 214 | 87.8 | 17.2 | 0.942 | 0.303 |
| | | | | Median | 1370 | 672 | 504 | 297 | 109 | 27.2 | 2.62 | 0.917 |
| | | | | Max | 1860 | 757 | 538 | 332 | 149 | 51.4 | 8.10 | 3.80 |

TABLE 33
Plasma Bumetanide Dibenzylamide Concentrations in Monkey
Following Sublingual Administration of 30 mg/kg Bumetanide Dibenzylamide (Fourth
Formulation).

| | | | | | Time | | | | | | | | |
|---|---|---|---|---|---|---|---|---|---|---|---|---|---|
| | | | | | (h) | | | | | | | | |
| | | | | | 0.00 | 0.25 | 0.50 | 1.0 | 2.0 | 4.0 | 8.0 | 24.0 | 32.0 |
| | | Dose | | | Bumetanide Dibenzylamide | | | | | | | | |
| Form | Route | (mg/kg) | Animal | Sex | (ng/mL) | | | | | | | | |
| 2 | SL | 5 | 23153 | M | 0.00 | 0.00 | 0.00 | 0.00 | 0.00 | 0.00 | 0.380 | 0.363 | 0.00 |
| | | | 23154 | M | 0.00 | 0.00 | 0.00 | 0.00 | 0.621 | 0.457 | 0.629 | 0.629 | 0.321 |
| | | | 23155 | F | 0.00 | 0.00 | 0.00 | 0.00 | 0.00 | 0.00 | 0.00 | 0.00 | 0.00 |
| | | | 23156 | F | 0.00 | 0.00 | 0.00 | 0.00 | 0.00 | 0.00 | 0.632 | 0.00 | 0.00 |
| | | | | N | 4 | 4 | 4 | 4 | 4 | 4 | 4 | 4 | 4 |
| | | | | Mean | 0.00 | 0.00 | 0.00 | 0.00 | 0.155 | 0.114 | 0.410 | 0.248 | 0.0803 |
| | | | | SD | 0.00 | 0.00 | 0.00 | 0.00 | 0.311 | 0.229 | 0.298 | 0.306 | 0.161 |
| | | | | CV % | | | | | 200.0 | 200.0 | 72.6 | 123.5 | 200.0 |
| | | | | Min | 0.00 | 0.00 | 0.00 | 0.00 | 0.00 | 0.00 | 0.00 | 0.00 | 0.00 |
| | | | | Median | 0.00 | 0.00 | 0.00 | 0.00 | 0.00 | 0.00 | 0.505 | 0.182 | 0.00 |
| | | | | Max | 0.00 | 0.00 | 0.00 | 0.00 | 0.621 | 0.457 | 0.632 | 0.629 | 0.321 |

### EXAMPLES

### Example 1: Synthesis of Bumetanide Dibenzylamide

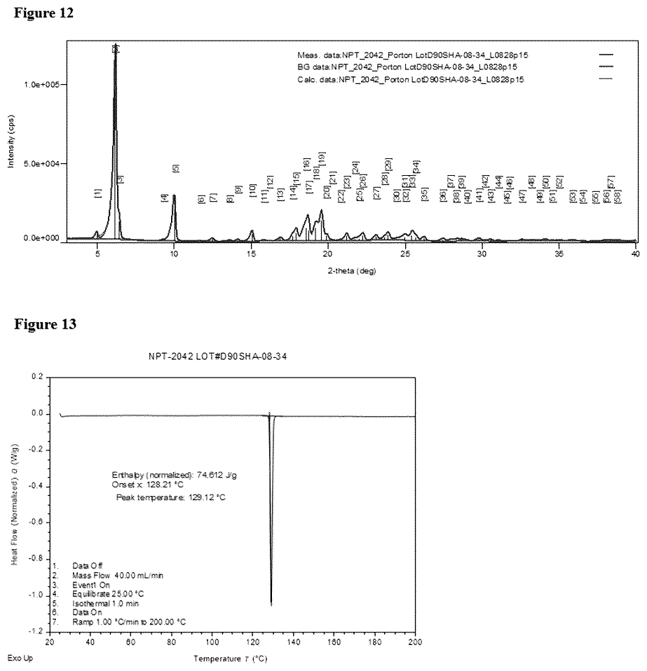

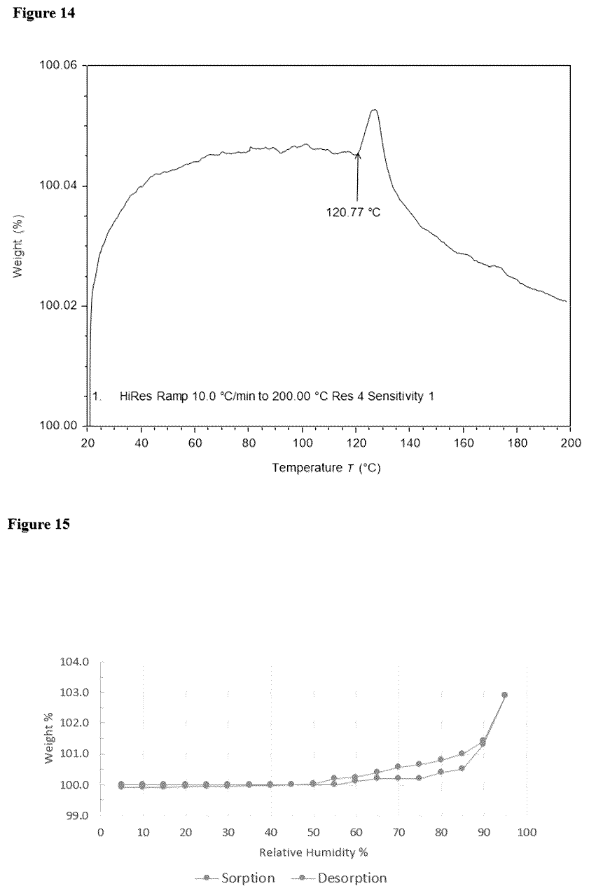

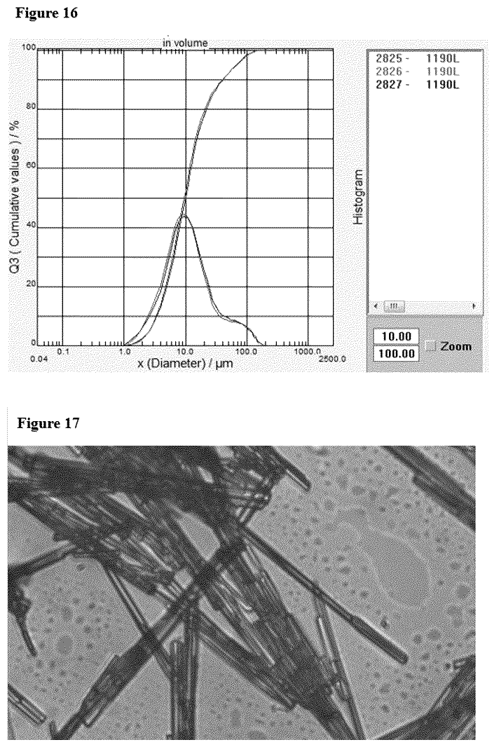

Bumetanide Dibenzylamide was synthesized as described below. Bumetanide (960 mg, 2.6 mmol) was dissolved in dimethyformamide (DMF, 10 mL) and 1-ethyl-3-(3-dimethy-laminopropyl) carbodiimide (EDC, 560 mg, 3.6 mmol) was added. After about 10 minutes, 1-hydroxybenzotriazole (HOBt, 392 mg, 2.9 mmol) was added and the solution was stirred for an additional 10 minutes. Dibenzylamine (1 mL, 5.2 mmol) was added and the reaction was stirred for 2 hours, at which time the reaction was complete by LC/MS. The reaction was poured into saturated ammonium chloride (20 mL) and extracted with ethyl acetate (2×100 mL). The ethyl acetate, was washed with saturated sodium bicarbonate, water, and brine, and dried over anhydrous magnesium sulfate. The ethyl acetate was removed under reduced pressure to yield 1.0 g (75%) of N, N-dibenzyl 3-aminosulfonyl-5-butylamino-4-phenoxybenzamide (Bumetanide Dibenzylamide) as white solid. FIG. 12 shows the X-ray diffractogram analysis of bumetanide dibenzylamide. FIG. 13 shows the DSC analysis of bumetanide dibenzylamide. FIG. 14 shows the TGA analysis of bumetanide dibenzylamide. FIG. 15 shows the DVS analysis of bumetanide dibenzylamide. FIG. 16 shows the PSD analysis of bumetanide dibenzylamide. FIG. 17 shows the morphology of Bumetanide Dibenzylamide under 40× magnification.

### Example 2: Oral Capsule

### Solubility Evaluation

The development of the oral capsule formulation was conducted in two parts. First, the basic solubility of Bumetanide Dibenzylamide in various common solubilizers was determined. Then compositions were constructed to obtain a self-emulsifying drug delivery system (SEDDS) using a mixture of medium chain triglycerides, long chain triglycerides, solubilizers, surfactants, and co-solvents.

TABLE 34
Common SEDDS excipients

| | | Range | |
|---|---|---|---|
| Class/Uses | Excipient examples | (% w/w) | |
| Non-ionic | Caprylocaproyl polyoxylglycerides (Labrasol ALF and | 10 | 45 |
| surfactant/ | Sopftigen), Lauroyl polyoxylglycerides (Gelucire 44/14 | | |
| solubilizer, | or 48/16), Linoleoyl polyoxylglycerides (Labrafil | | |
| emulsifying | M2125CS), Oleoyl polyoxylglycerides (Labrafil | | |
| agent | M1944CS), Stearoyl polyoxylglycerides (Gelucire 50/ | | |
| | 13), Polyoxyl 35 Castor Oil (Kolliphor EL) or | | |
| | Cremophor RH40 (polyoxyl castor oil), vitamin E | | |
| | TPGS, Span 20 (Sorbitan oleate), Tween 20 or 80 | | |
| | (polysorbate), Poloxamers, Solutol HS-15, | | |
| Oil Phase | Arachis oil, soybean oil, castor oil, corn oil, safflower | 15 | 65 |
| | oil, olive oil, apricot kernel oil, sesame oil, cotton seed | | |
| | oil, sunflower seed oil, palm seed oil, coconut oil, | | |
| | rapeseed oil, peanut oil,p eppermint oil, hydrogenated | | |
| | soybean oil, hydrogenated vegetable oils, Maisine | | |
| | 35-1 or Maisine CC or Peceol (glyceryl monolinoleate), | | |
| | Beeswax, oleic acid, soy fatty acids, Vitamin E, | | |
| | Labrafac, Miglyol, Crodamol, Capmuyl, Imwitor, | | |
| | Captex and other medium chain triglycerides (MCTs) | | |
| Co-solvent | Propylene Glycol, Capryol ™ 90, Lauroglycol ™ 90, | 0 | 15 |
| | Glycerin, Polyethylene glycol, ethanol, tetraglycol | | |
| Antioxidant | alpha tocopherol, ascorbyl palmitate, ascorbic acid, | 0 | 0.15 |
| | butylatedhydroxyanisole or butylated hydroxytoluene | | |

The excipients listed in Table 34 are proposed based on the functional category and prior history of use. The ranges provided are estimates based on the available data obtained during the initial development, prior experience, and literature research. These ranges represent the space in which a suitable self-emulsifying drug delivery system may be obtained in combination with the other excipients in ratios adjusted to total 100% w/w.

The solubility of Bumetanide Dibenzylamide at 50° C. was determined in multiple solubilizers by visual inspection in common SEDDS excipients. Bumetanide Dibenzylamide was added stepwise to 2 grams of solubilizer that was heated to 50° C. and allowed to stir at 800 RPM on a stir plate until the Bumetanide Dibenzylamide no longer dissolved in a 10-minute period. The results are presented in Table 35. Bumetanide Dibenzylamide had the highest solubility in Lauroyl Plyoxyl-32 Glycerides (Gelucire 44/14), Caprylocaproyl Polyoxylglycerides (Labrasol ALF), Sorbitan Ester (Span 80), and Polyoxyl 35 Castor Oil (Kolliphor EL), and thus, these were chosen as candidate solubilizers in SEDDS development.

TABLE 35
Bumetanide Dibenzylamide solubility
in common SEDDS excipients

| | Bumetanide |
|---|---|
| | Dibenzylamide, in |
| Excipient/Solubilizer | solution (mg/ml |
| Kolliphor EL (Polyoxyl 35 | 13.5 |
| castor oil) | |
| Captex 300 (Caprylic/Capric | <1* |
| Triglyceride, Medium Chain | |
| Triglyceride) | |
| Labrasol ALF | 63 |
| (Caprylocaproyl | |
| Polyoxylglycerides) | |
| Labrafac Lipophile WL 1349 | 3 |
| (Medium Chain Triglyceride) | |
| Drakeol 6VR LT (Mineral Oil) | <1* |
| Labrafil M 1944CS (Oleoyl | 4.5 |
| polyoxyl 1-6 glycerides) | |
| Propylene Glycol | 2 |
| Soybean Oil | 2 |
| Oleyl Alcohol | <1* |
| Lauroyl Plyoxyl-32 | 106 |
| Glycerides (Gelucire 44/14) | |
| Sorbitan Ester (Span 80) | 250 |
| Oleic Acid | <1* |
| Polyethylene Glycol 400 | 8.6 |

* = Did not visually go into solution

### Capsule Development and Analysis

Prototype capsule formulations were developed at a less than 2-gram scale. The solubilizers/surfactants were heated up to 60° C. while stirring at ˜800 RPM using a magnetic stir bar and adding Bumetanide Dibenzylamide until it dissolved. The oil phase was then added to the formulations and stirred until the solution became clear. The solutions were cooled to room temperature while stirring and co-solvent was then added (if applicable). Compositions containing long chain triglycerides and increasing amounts of ethanol as a co-solvent in the range (0 to 4.68% w/w) with the maximum solubility of Bumetanide Dibenzylamide determined to be about 1.8% w/w were investigated as illustrated in Table 37 and Table 38. The comparator compositions are listed in Table 36. For complete solubilization of Bumetanide Dibenzylamide, the minimum amount of required ethanol was determined to be about 3.24% (composition 6).

TABLE 36
Prototype capsule compositions

| | Composition 1 | | | Composition 2 | | | Composition 3 | | |
|---|---|---|---|---|---|---|---|---|---|
| Excipient | % w/w | mg/cap | grams | % w/w | mg/cap | grams | % w/w | mg/cap | grams |
| Bumetanide | 1.59 | 10.03 | 0.0318 | 1.98 | 10.00 | 0.032 | 1.98 | 10.00 | 0.032 |
| Dibenzylamide | | | | | | | | | |
| Phosphatidylcholine | 22.00 | 138.60 | 0.4400 | | | | | | |
| (Phosal 75 SA) | | | | | | | | | |
| Caprylocaproyl | 70.00 | 441.00 | 1.4000 | | | | 39.72 | 200.94 | 0.643 |
| Polyoxylglycerides | | | | | | | | | |
| (Labrasol ALF) | | | | | | | | | |
| Lauroyl Plyoxyl-32 | | | | 19.39 | 98.13 | 0.314 | | | |
| Glycerides | | | | | | | | | |
| (Gelucire 44/14) | | | | | | | | | |
| Sorbitan Ester | | | | 37.62 | 190.31 | 0.609 | 25.94 | 131.25 | 0.420 |
| (Span 80) | | | | | | | | | |
| Caprylic/Capric | 6.41 | 40.37 | 0.1282 | | | | | | |
| Triglyceride | | | | | | | | | |
| (Captex 300) | | | | | | | | | |
| Soybean oil | | | | 41.01 | 207.50 | 0.664 | 49.78 | 251.88 | 0.806 |
| Total | 100.00 | 630.00 | 2.00 | 100.00 | 505.94 | 1.62 | 117.42 | 505.94 | 1.90 |

TABLE 37
Prototype capsule compositions

| | Composition 4 | | | Composition 5 | | |
|---|---|---|---|---|---|---|
| Excipient | % w/w | mg/cap | grams | % w/w | mg/cap | grams |
| Bumetanide | 1.79 | 11.25 | 0.032 | 1.76 | 11.06 | 0.032 |
| Dibenzylamide | | | | | | |
| Polyoxyl 35 | 33.48 | 210.94 | 0.600 | 32.93 | 207.46 | 0.600 |
| Castor Oil | | | | | | |
| (Kolliphor EL) | | | | | | |
| Glyceryl | 32.37 | 203.91 | 0.580 | 31.83 | 200.55 | 0.580 |
| Monolinoleate | | | | | | |
| (Maisine CC) | | | | | | |
| Soybean oil | 32.37 | 203.91 | 0.580 | 31.83 | 200.55 | 0.580 |
| Ethanol | | | | 1.65 | 10.37 | 0.030 |
| Total | 100.00 | 630.00 | 1.79 | 100.00 | 630.00 | 1.82 |

TABLE 38
Prototype capsule compositions

| | Composition 6 | | | Composition 7 | | |
|---|---|---|---|---|---|---|
| Excipient | % w/w | mg/cap | grams | % w/w | mg/cap | grams |
| Bumetanide | 1.73 | 10.89 | 0.032 | 1.70 | 10.72 | 0.032 |
| Dibenzylamide | | | | | | |
| Polyoxy1 35 | 32.40 | 204.10 | 0.600 | 31.91 | 201.06 | 0.600 |
| Castor Oil | | | | | | |
| (Kolliphor | | | | | | |
| EL) | | | | | | |
| Glyceryl | 31.32 | 197.30 | 0.580 | 30.85 | 194.36 | 0.580 |
| Monolinoleate | | | | | | |
| (Maisine CC) | | | | | | |
| Soybean oil | 31.32 | 197.30 | 0.580 | 30.85 | 194.36 | 0.580 |
| Ethanol | 3.24 | 20.41 | 0.060 | 4.68 | 29.49 | 0.088 |
| Total | 100.00 | 630.00 | 1.85 | 100.00 | 630.00 | 1.88 |

To test if the composition created macro-dispersions or micro-dispersions, about 30 mg of formulation was added into 1.0 g of water and lightly swirled. The resultant dispersion was visually characterized. SEDDS (macroemulsions) are typically characterized as an opaque milky white uniform liquid, and SEDDS (microemulsions) are characterized by a clear, isotropic, transparent solution with opalescence. Compositions 4-6 created a functioning SEDDS for Bumetanide Dibenzylamide. Compositions 4 and 6 were selected for future development work, and the intermediate stability of those compositions was evaluated as described below.

### Intermediate Stability

To determine the chemical stability over a short-term duration, compositions 4 (Table 37) and 6 (Table 38) were scaled to a 100 g batch size, encapsulated and stored in 40° C./75% RH conditions for 2 weeks. The antioxidant, butylated hydroxytoluene (BHT) was added to each composition at 0.03% w/w.

### Stability and Storage Analysis

The capsules were manually packaged into HDPE bottles and heat sealed. There was no significant change in the appearance of the capsules over the first 2 weeks of the stability study as shown in Table 39.

TABLE 39
Appearance Results

| | Time | | |
|---|---|---|---|
| Batch | Point | Appearance Specification | Results |
| Com- | Initial | Off-white capsule with | Conforms |
| position 6 | Weeks | no visible breaks, leaks | Conforms |
| 40° C./ | 2 | or defects on the surface | |
| 75% RH | | | |
| Com- | Initial | Off-white capsule with | Conforms |
| position 4 | 2 Weeks | no visible breaks, leaks | Conforms |
| 40° C./ | | or defects on the surface | |
| 75% RH | | | |

The results for assay and related substances are illustrated in Table 40. From these results, it is evident that both drug product lots: compositions 4 and 6, do not produce a significant amount of degradation products when stored at 40° C./75% RH conditions.

TABLE 40
Assay and Related Substance results

| | | Composition 6 | | Composition 4 | |
|---|---|---|---|---|---|
| Formulation | | T = | T = | T = | T = |
| Peak ID | | 0 wks | 2 wks | 0 wks | 2 wks |
| UNK RRT | 0.803 | 0.086 | 0.126 | 0.081 | 0.117 |
| UNK RRT | 0.861 | | 0.056 | | |
| UNK RRT | 0.901 | | 0.064 | | 0.063 |
| UNK RRT | 0.932 | | 0.106 | | 0.096 |
| Assay RRT | 1.0 | 104.9 | 101.4 | 101.9 | 101.4 |
| UNK RRT | 1.064 | 0.116 | 0.111 | 0.121 | 0.112 |
| UNK RRT | 1.082 | 1.002 | 1.016 | 1.045 | 1.085 |
| UNK RRT | 1.095 | 0.121 | 0.09 | 0.13 | 0.131 |
| UNK RRT | 1.112 | 2.831 | 0.072 | | |
| UNK RRT | 1.166 (BHT) | | 2.819 | | 2.86 |
| UNK RRT | 1.184 | | 0.059 | 2.917 | 0.057 |
| UNK RRT | 1.191 | | | 0.051 | 2.14 |
| UNK RRT | 1.224 | | 2.174 | 0.262 | |

| | | | | |
|---|---|---|---|---|
| Total % Area | 4.2 | 6.7 | 4.6 | 6.7 |
| Total % Area - Placebo | 0.145 | 2.649 | 0.378 | 2.514 |

Based on this data, formulation Capsule of composition 6 with about 3.24% Ethanol (shown in Table 38) was selected for use in the animal studies. As shown in Table 41, Bumetanide Dibenzylamide concentration may be adjusted from about 0 to about 1.75% w/w with corresponding adjustment of Polyoxyl 35 Castor Oil (Kolliphor EL) % w/w while maintaining ratio of all other excipients for a total weight of 100%.

TABLE 41
Bumetanide Dibenzylamide Capsule Composition

| | Composition 6 | | |
|---|---|---|---|
| Excipient | mg/cap | % w/w | grams |
| Bumetanide Dibenzylamide | 10.03 | 1.75 | 1.750 |
| Polyoxyl 35 Castor Oil | 185.45 | 32.36 | 32.365 |
| (Kolliphor EL) | | | |
| Glyceryl Monolinoleate | 179.35 | 31.30 | 31.300 |
| (Maisine CC) | | | |
| Soybean oil | 179.35 | 31.30 | 31.300 |
| Ethanol | 18.62 | 3.25 | 3.250 |
| Butylated hydroxytoluene | 0.20 | 0.03 | 0.035 |
| Total | 573.00 | 100.00 | 100.00 |

### Example 3: Nasal Solution

### Solubility Evaluation

A study was conducted to evaluate the solubility of Bumetanide Dibenzylamide in various solvent/co-solvent systems (Table 43). The solvents were chosen from a group consisting of Caprylocaproyl Polyoxylglycerides (Labrasol ALF), propylene glycol, PEG-400, vitamin E TPGS, ethanol, water, and any combination thereof.

The excipients listed in Table 42 are proposed based on the functional category and prior history of use. The ranges provided are estimates based on the available data obtained during the initial development, prior experience, and literature research. These ranges represent the space in which a suitable solution for nasal delivery may be obtained in combination with the other excipients in ratios adjusted to total 100% w/w.

TABLE 42
Common excipients for nasal solution

| | | Range | |
|---|---|---|---|
| Class/Uses | Excipient examples | (% w/w) | |
| Non-ionic | Caprylocaproyl polyoxylglycerides (Labrasol | 1 | 80 |
| surfactant/ | ALF), Lauroyl polyoxylglycerides (Gelucire | | |
| solubilizer, | 44/14 or 48/16), Linoleoyl polyoxylglycerides | | |
| emulsify- | (Labrafil M2125CS), Oleoyl | | |
| ing agent | polyoxylglycerides (Labrafil M1944CS), | | |
| | Stearoyl polyoxylglycerides (Gelucire 50/13), | | |
| | Kolliphor EL (polyoxyl 35 castor oil), | | |
| | vitamin E TPGS, Span 20, 40, 80 (Sorbitan | | |
| | oleate), Tween 20 or 80 (polysorbate) | | |
| Co-solvent | Propylene Glycol, Capryol ™ 90, | 0 | 80 |
| | Lauroglycol ™ 90, Glycerin, Polyethylene | | |
| | Glycol, ethanol | | |
| Diluent | Water | 5 | 100 |

Based on the solubility data and the chemical properties of Bumetanide Dibenzylamide, a low percentage of water is required to achieve reasonable solubility. Therefore, PEG-400 was selected as the primary diluent.

TABLE 43
Solubility Evaluation Experimental Design and Composition Summary

| | Solvent System (% w/w) | | | | | |
|---|---|---|---|---|---|---|
| | Caprylocaproyl | | | | | |
| | Polyoxylglycerides | Propylene | | Vitamin E | | |
| Run | (Labrasol ALF) | Glycol | PEG-400 | TPGS | Ethanol | Water |
| Composition 8 | 100.00 | | | | | 0.00 |
| Composition 9 | 50.00 | | | | | 50.00 |
| Composition 10 | | 12.00 | 80.00 | | | 8.00 |
| Composition 11 | 10.00 | 12.00 | 70.00 | | | 8.00 |
| Composition 12 | | 20.00 | 70.00 | 1.00 | | 9.00 |
| Composition 13 | 10.00 | 20.00 | 57.00 | 1.00 | 2.00 | 10.00 |

50 g stock solutions of each solvent system were prepared. The active solutions 3.0 mL of each solvent was aliquoted to separate 8 mL clear glass vials. 30 mg±2 of Bumetanide Dibenzylamide was added to each vial and the samples are mixed on a wrist action shaker for 8 hours. Every 30 minutes the solutions were inspected. If no visible Bumetanide Dibenzylamide was present, an additional 10 mg was added and the mixing was continued. This process was repeated until visible saturation of all solvent systems was obtained. Prior to analysis, the samples were centrifuged at 5,000 RPM for 3 minutes and filtered through a 0.45 μm nylon syringe filter to remove excess solids. The samples were quantitated using an HPLC method with UV absorbance detection Sample measurement was performed in triplicate, and the average results was used to determine the solubility of Bumetanide Dibenzylamide in each system.

Comparison of the results indicated that the solubility of the drug substance might be significantly affected by the amount of water in the system (Table 44). While Bumetanide Dibenzylamide readily dissolved in Caprylocaproyl Polyoxylglycerides (Labrasol ALF), up to ˜66 mg/mL (Composition 8), the solubility was reduced by slightly less than 10× in 50:50 Caprylocaproyl Polyoxylglycerides (Labrasol ALF), and water. The solubility appears to decrease in correlation to each % w/w increase of water in compositions 11-13. When sufficient solubility was achieved, the composition was evaluated for use as a nasal spray. The solubility of Bumetanide Dibenzylamide in 100% Propylene Glycol (PEG 400) and 100% Caprylocaproyl Polyoxylglycerides (Labrasol ALF), (Composition 8) was also evaluated.

TABLE 44
Summary of Solubility Results and Estimated Dose Calculations

| | μg/ | Dil. Vol. | Dil | mg/ | 75% | per dose | |
|---|---|---|---|---|---|---|---|
| Run | mL¹ | (mL) | (mL) | mL² | Sat | 0.4 mL | 1.0 mL |
| Composition 8 | 796.6 | 0.3 | 25.0 | 66.4 | 49.8 | 19.9 | 49.8 |
| Composition 9 | 240.9 | 1.0 | 25.0 | 6.0 | 4.5 | 1.8 | 4.5 |
| Composition 10 | 497.4 | 1.0 | 25.0 | 12.4 | 9.3 | 3.7 | 9.3 |
| Composition 11 | 832.0 | 0.5 | 25.0 | 41.6 | 31.2 | 12.5 | 31.2 |
| Composition 12 | 633.8 | 0.5 | 25.0 | 31.7 | 23.8 | 9.5 | 23.8 |
| Composition 13 | 485.3 | 0.5 | 25.0 | 24.3 | 18.2 | 7.3 | 18.2 |
| 100% PEG | 342.8 | 1.0 | 25.0 | 8.6 | 6.4 | 2.6 | 6.4 |
| 400 | | | | | | | |

¹measured concentration in the analytical dilution
²concentration of Bumetanide Dibenzylamide in the solvent system

Saturated solubility of Bumetanide dibenzylamide in the compositions described in Table 42 are in the range of about 0.6% to about 6.64%. The dose calculations included in Table 42 are based on 75% of the concentration at saturation. This is to reduce the risk of precipitation/crystallization due to environmental fluctuations. Additionally, two dose volumes (0.2/0.5 mL) were selected in accordance with the standard range for nasal delivery. The total dose illustrated is based on the equivalent volume delivered to each nostril, 0.4-1.0 mL total per dose.

A primary solvent system based on composition-11 was selected for further evaluation. To maximize the amount of water in the formulation an additional set of 3 samples were prepared based on this composition and the effect of the water content from about 10% w/w to about 20% w/w was evaluated.

### Effect of Water Content

The amount of water in the formulation may have a significant impact on the solubility of Bumetanide Dibenzylamide. For this reason, 3 additional small scale formulations (F1-F3) were prepared according to the compositions presented in Table 45. Increasing amounts of PVP and Poloxamer were added to compositions F1-F3 to inhibit rapid precipitation upon dilution in water.

TABLE 45
Effect of Water Content - Formulation Compositions

| Item | | % w/w | | |
|---|---|---|---|---|
| No. | Ingredient | F1 | F2 | F3 |
| 1 | Bumetanide Dibenzylamide | Excess | Excess | Excess |
| 2 | Caprylocaproyl | 10.00 | 10.00 | 10.00 |
| | Polyoxylglycerides | | | |
| | (Labrasol ALF) | | | |
| 3 | Propylene Glycol | 9.15 | 9.15 | 9.15 |
| 4 | PEG-400 | 62.58 | 59.58 | 52.58 |
| 5 | Polyvinylpyrrolidone (PVP) | 3.00 | 2.50 | 3.00 |
| 6 | Poloxamer 407 (P407) | 3.00 | 2.50 | 3.00 |
| 7 | Purified Water | 10.00 | 14.00 | 20.00 |
| | Total | 100.00 | 100.00 | 100.00 |

An excess of Bumetanide Dibenzylamide was added to each formulation while mixing at 40-45° C. The solutions were then cooled to ambient room temperature and mixed for ˜4 hours prior to filtering and being evaluated by an assay.

The solubility data for this evaluation was compared to the results obtained from composition-11 that had a similar composition without PVP and Poloxamer (Table 46). These results support the hypothesis that the solubility of Bumetanide Dibenzylamide decreases non-linearly as the water content increases, or as the water:organic solvent ratio changes.

TABLE 46
Results for Effect of Water Content

| | % w/w | mg/ml in |
|---|---|---|
| Formulation | water | Solution |
| Composition | 8 | 41.6 |
| 11 | | |
| F1 | 10 | 30.4 |
| F2 | 14 | 25.3 |
| F3 | 20 | 23.9 |

The solubility of Bumetanide Dibenzylamide was evaluated and a solvent or solvent system that results in the highest possible concentration (mg/mL) of the composition in solution was identified. The solutions were found to comprise from about 20 mg/mL to about 40 mg/mL of Bumetanide Dibenzylamide. The results show that the optimum composition (bumetanide dibenzylamide concentration of at least 30 mg/mL) may comprise water in the range from about 0% w/w to about 10% w/w. Two separate compositions were selected for short-term stability analysis.

### Intermediate Stability

Based on the results of the solubility study, a short-term stability assessment was conducted for two proposed compositions of Bumetanide Dibenzylamide nasal solution. Formulation of the composition of the nasal solution was based on solubility evaluation.

The concentration of Bumetanide Dibenzylamide in the solution was about 30 mg/mL. The short-term stability assessment was performed over two weeks accelerated storage (40° C./75% RH). Testing was conducted at time zero (T=0) and 2 weeks. An overview of the compositions are illustrated in Tables 47-48.

TABLE 47
First Formulation - Nasal
Solution 30 mg/mL Composition-A

| Item# | Ingredient | % w/w | % w/v | mg/ml |
|---|---|---|---|---|
| 1 | Bumetanide | 2.73 | 3.00 | 30.00 |
| | Dibenzylamide | | | |
| 2 | Caprylocaproyl | 10.00 | 11.00 | 110.00 |
| | Polyoxylglycerides | | | |
| | (Labrasol ALF) | | | |
| 3 | Propylene Glycol | 12.05 | 13.26 | 132.60 |
| 4 | Polyethylene Glycol | 67.22 | 73.94 | 739.40 |
| | 400 | | | |
| 5 | Water | 8.00 | 8.80 | 88.00 |
| | Total | 100.00 | 110.00 | 1100.00 |

TABLE 48
Second Formulation - Nasal
Solution 30 mg/mL Composition-B

| Item# | Ingredient | % w/w | % w/v | mg/ml |
|---|---|---|---|---|
| 1 | Bumetanide | 2.82 | 3.13 | 31.27 |
| | Dibenzylamide | | | |
| 2 | Caprylocaproyl | 4.65 | 5.17 | 51.67 |
| | Polyoxylglycerides | | | |
| | (Labrasol ALF) | | | |
| 3 | Propylene Glycol | 13.01 | 14.44 | 144.44 |
| 4 | Polyethylene Glycol 400 | 67.97 | 75.45 | 754.49 |
| 5 | Polyvinylpyrrolidone | 0.92 | 1.03 | 10.25 |
| | (K30) | | | |
| 6 | Water | 10.62 | 11.79 | 117.89 |
| | Total | 100.00 | 111.00 | 1110.00 |

The prepared solutions were filtered through a 0.45 μm nylon filter and packaged into clear glass vials. The samples were stored upright in glass serum vials at 40° C./75% RH for 2 weeks. Analysis was performed using an HPLC method with UV detection.

There were no observable changes in the samples at the end of the study, and no visible precipitation/particulates. The results (Table 49) demonstrated that the change in assay for a composition was less than the analytical precision (+2.0%).

TABLE 49
Nasal Solution Stability - Assay Results

| | % Assay of | | |
|---|---|---|---|
| | Bumetanide | | |
| | Dibenzylamide | | % |
| Batch # | T = 0 | T = 2 Wks | Diff |
| Composition | 104.8 | 103.8 | 1.0 |
| A | | | |
| Composition | 105.0 | 98.2 | 6.8 |
| B | | | |

The data for related compounds are reported as percent area relative to the Bumetanide Dibenzylamide peak in the sample (Table 50). These results did not indicate any significant individual or total amount of impurity at time 0 (T=0) or 2 week accelerated stability.

TABLE 50
Related Compounds Results

| Impurity | Composition A | | Composition B | |
|---|---|---|---|---|
| (by RRT) | T = 0 | T = 2Wks | T = 0 | T = 2Wks |
| 0.67 | 0.05 | <LOQ | <LOQ | <LOQ |
| 0.78 | 0.17 | 0.14 | 0.07 | 0.07 |
| 0.81 | 0.11 | 0.15 | 0.11 | 0.12 |
| 0.86 | — | <LOQ | — | <LOQ |
| 1.03 | <LOQ | <LOQ | <LOQ | <LOQ |
| Total Imp % | 0.33 | 0.29 | 0.18 | 0.19 |

— Not Detected

Both compositions demonstrated suitable short-term stability as it pertained to the appearance and related compounds. Based on these data composition A was selected for use in the animal studies. For composition A, Bumetanide dibenzylamide concentration may be adjusted from about 20 mg/mL to about 40 mg/mL (2 to 4%) with corresponding range of water content 8 to 10% maintaining ratio of all other excipients for a total weight of 100%.

### Example 5: Rectal Gel and Rectal Paste

Two separate variations were prepared from the nasal solution solvent system to produce a gel (primary formulation) and paste (alternative formulation). Ranges for excipients as shown in Table 51 are the same as for the nasal solution with additional gelling and thickening agents, and water is limited to about 20%. Since a rectal gel formulation comprises about 20% water, the maximum concentration of Bumetanide Dibenzylamide in a rectal gel is about 2.4%. Since a rectal paste formulation does not comprise water, the maximum concentration of Bumetanide Dibenzylamide in a rectal paste is about 4.16%.

TABLE 51
Common excipients for rectal gel and paste

| | | Range | |
|---|---|---|---|
| Class/Uses | Excipient examples | (% w/w) | |
| Non-ionic | Caprylocaproyl polyoxylglycerides (Labrasol ALF), Lauroyl | 1 | 80 |
| surfactant/ | polyoxylglycerides (Gelucire 44/14 or 48/16), Linoleoyl | | |
| solubilizer, | polyoxylglycerides (Labrafil M2125CS), Oleoyl polyoxyl- | | |
| emulsifying | glycerides (Labrafil M1944CS), Stearoyl polyoxylglycerides | | |
| agent | (Gelucire 50/13), Kolliphor EL (polyoxyl 35 castor oil), | | |
| | vitamin E TPGS, Span 20, 40, 80 (Sorbitan oleate), Tween | | |
| | 20 or 80 (polysorbate) | | |
| Co-solvent | Propylene Glycol, Capryol ™ 90, Lauroglycol ™ 90, | 0 | 80 |
| | Glycerin, Polyethylene Glycol, ethanol | | |
| Diluent | Water | 0 | 20 |
| Gelling and | Carbomer, Pemulen (synthetic polymers of acrylic acid) | 0.4 | 10 |
| Thickening | Hydroxypropyl cellulose, carboxymethylcellulose, | | |
| agent | hydroxyethyl cellulose, ethylcellulose, xylulose (cellulose | | |
| | based polymers) | | |
| | Xanthan gum, gellan gum, guar gum, pectin, gelatin, starch, | | |
| | alginate, tragacanth, bentonite, chitosan, carrageenan, acacia | | |
| | (natural polymers) | | |
| | Polyvinyl alcohol, polyethylene oxide, Poloxamer or Pluronic, | | |
| | polyvinyl pyrrolidone or povidone, Polyethylene glycol 3350 | | |
| | (synthetic polymers) | | |

For the rectal gel (Table 52), a separate solution of sodium carboxymethyl cellulose (CMC) was prepared and combined with the non-aqueous phase to produce a clear-to-slightly opaque, viscous gel. The amount of CMC selected was based on prior experience and the resulting gel was concluded to be suitable to support the initial animal studies.

TABLE 52
Rectal Gel Formulation Composition

| Item# | Ingredient | % w/w | mg/g |
|---|---|---|---|
| 1 | Bumetanide | 0.60 | 6.00 |
| | Dibenzylamide | | |
| 2 | Caprylocaproyl | 10.00 | 100.00 |
| | Polyoxylglycerides | | |
| | (Labrasol ALF) | | |
| 3 | Propylene Glycol | 9.15 | 91.50 |
| 4 | Polyethylene Glycol 600 | 53.84 | 538.42 |
| 5 | Polyvinylpyrrolidone | 3.60 | 36.00 |
| | (K30) | | |
| 6 | Poloxamer 407 (P407) | 2.40 | 24.00 |
| 7 | Sodium CMC | 0.41 | 4.08 |
| 8 | Purified Water | 20.00 | 200.00 |
| | Total | 100.00 | 1000.00 |

For the initial formulation for the rectal paste, a stock solution of the solvent phase (items 1-6 in Table 53) was prepared. PEG-3350 was added stepwise/quantitatively to this solution until a semi-solid paste was obtained, ˜7.5% w/w. The amount of Bumetanide Dibenzylamide in the formulation was then adjusted to obtain the target concentration (6 mg/g).

TABLE 53
Rectal Paste Formulation Composition

| Item# | Ingredient | % w/w | mg/g |
|---|---|---|---|
| 1 | Bumetanide | 0.60 | 6.00 |
| | Dibenzylamide | | |
| 2 | Caprylocaproyl | 10.00 | 100.00 |
| | Polyoxylglycerides | | |
| | (Labrasol ALF) | | |
| 3 | Propylene Glycol | 9.15 | 91.50 |
| 4 | Polyethylene Glycol 600 | 66.75 | 667.50 |
| 5 | Polyvinylpyrrolidone | 3.60 | 36.00 |
| | (K30) | | |
| 6 | Poloxamer 407 (P407) | 2.40 | 24.00 |
| 7 | Polyethylene Glycol 3350 | 7.50 | 75.00 |
| | Total | 100.00 | 1000.00 |

### Intermediate Stability

The gel and paste formulations were then analysed for short-term stability. A short-term stability assessment was conducted for two proposed formulations of the Bumetanide Dibenzylamide rectal dose 6 mg/mL. This study was performed over two weeks accelerated storage (40° C./75% RH).

Each formulation batch was prepared by weight and about 50 g was packaged in 5 separate 4 oz jars with PTFE lined caps for stability/storage.

### Stability Storage and Analysis

The samples were stored upright at 40° C./75% RH for 2 weeks. Analysis was performed using an HPLC method with UV detection. The appearance of the rectal gel was viscous, clear to opaque colorless gel. The appearance of the rectal paste was viscous/flowable white-to-off-white opaque paste.

There were no observable changes in the appearance of (Table 54) the samples at the end of the study, and no visible precipitation/particulates. Both compositions were determined to be close to 100% of the expected assay of (6 mg/g). But, at 2 weeks, there was a slight decrease in the assay of the rectal paste. Also, the gel formulation exhibited a significant drop in the assay value. However, there was no observed increase in the related compounds. Therefore, it is possible that the change in assay may be due to non-uniformity resulting from precipitation of the Bumetanide Dibenzylamide. Also due to the hygroscopicity of the preparations and absorbed water in the high humidity storage conditions may be the cause of the significant decrease in assay.

TABLE 54
Rectal Dose Stability - Results for Assay

| | % Assay of | | |
|---|---|---|---|
| | Bumetanide | | |
| | Dibenzylamide | | % |
| Batch # | T = 0 | T = 2 Wks | Diff |
| Composition | 99.8 | 97.4 | 2.4 |
| C | | | |
| Composition | 98.4 | 89.1 | 9.3 |
| D | | | |

There were no significant changes in the related compounds between T=0 and 2 weeks accelerated stability (Table 55). The only impurity detected above 0.10% at relative retention time (RRT) 0.86, was present in all of the samples, and did not change over the course of the study, so this impurity is not likely to be a degradant. From the results for the short-term stability study, composition C was selected for use in the animal studies.

TABLE 55
Rectal Dose Stability - Results for Related Compounds

| Impurity | Composition C | | Composition D | |
|---|---|---|---|---|
| (by RRT) | T = 0 | T = 2 Wks | T = 0 | T = 2 Wks |
| 0.80 | — | 0.05 | — | — |
| 0.86 | 0.35 | 0.33 | 0.35 | 0.33 |
| 1.17 | 0.08 | 0.07 | 0.08 | 0.06 |
| Total Imp | 0.43 | 0.45 | 0.43 | 0.39 |

— Not Detected

### Example 6: Tablet-Buccal or Sublingual Administration

Two tablet compositions were developed targeting a dose strength of up to about 30 mg Bumetanide Dibenzylamide per tablet or capsule. In some instances, the dose strength is about 10 mg Bumetanide Dibenzylamide per tablet. Solubility studies similar to those done for the oral capsule compositions, as described earlier, were done. The solubility studies identified both drug solubility and wettability to be of concern in aqueous media. Additionally, the oral cavity rarely contains more than about 2 ml of saliva available for solubilization/oral disintegration. These issues surrounding oral disintegration and solubilization were accounted for during the formulation process.

A traditional direct blend tablet complete with a wetting agent and solubilizer was developed. Disintegration was evaluated by continual immersion of the tablets with repeated vertical motion in approximately 700 ml of 37° C. water in a low form 1000 ml beaker until complete disintegration is observed.

TABLE 56
Common excipients for dry powder

| | | Range | |
|---|---|---|---|
| Class/Uses | Excipient examples | (% w/w) | |
| Diluent/ | Ceolus KG 802 (Microcrystalline Cellulose), Mannogem EZ (Spray | 50 | 90 |
| Filler | Dried Mannitol), Lactose, Xylitol, carboxymethylcellulose, starch, | | |
| | calcium phosphate, sucrose, dextrin, maltose, sorbitol, maltodextrin, | | |
| | calcium carbonate, hydroxypropylcellulose, ethylcellulose, | | |
| | hydroxypropyl methylcellulose | | |
| Binder | Polyvinylpyrrolidone, starch, sucrose, dextrin, maltose, sorbitol, | 0 | 10 |
| | polyvinylacetate, copovidone, maltodextrin, acacia, gum agar, | | |
| | carboxymethylcellulose, polymethyacrylate | | |
| Disintegrate | Polyplasdone XL (Super Disintegrate), microcrystalline cellulose, | 0 | 10 |
| | natural clays, aligns, gums and surfactants, sodium starch glycolate | | |
| Lubricant | Magnesium Stearate, talc, metal stearates, stearic acid, boric acid, | 0 | 2 |
| | sodium chloride, benzoate and acetate sodium or magnesium lauryl | | |
| | sulfate, carbowax, cornstarch, sodium lauryl sulfate | | |
| Substrate/ | Neusilin US2 (Magnesium Aluminometasilicate), Ceolus KG 802 | 0 | 50 |
| absorbant | (Microcrystalline Cellulose), dibasic calcium phosphate (Fujicalin), | | |
| | maltodextrin, ordered mesoporous silicates, Aerosil, calcium | | |
| | silicate, natural clays | | |
| Glidant | Cabosil M5P (Fumed Silica), colloidal silica, magnesium stearate, | 0 | 2 |
| | purified talc | | |
| Non-ionic | Caprylocaproyl polyoxylglycerides (Labrasol and Sopftigen), | 0 | 35 |
| surfactant/ | Lauroyl polyoxylglycerides (Gelucire 44/14 or 48/16), Linoleoyl | | |
| solubilizer, | polyoxylglycerides (Labrafil M2125CS), Oleoyl polyoxylglycerides | | |
| emulsifying | (Labrafil M1944CS), Stearoyl polyoxylglycerides (Gelucire 50/13), | | |
| agent, | Polyoxyl 35 Castor Oil (Kolliphor EL) or Cremophor RH40 | | |
| wetting | (polyoxyl castor oil), vitamin E TPGS, Span 20 (Sorbitan oleate), | | |
| agent | Tween 20 or 80 (polysorbate), Poloxamers, Solutol HS-15, | | |
| Solubilizer/ | Arachis oil, soybean oil, castor oil, corn oil, safflower oil, olive oil, | 0 | 35 |
| Fixed Oil | apricot kernel oil, sesame oil, cotton seed oil, sunflower seed oil, | | |
| | palm seed oil, coconut oil, rapeseed oil, peanut oil, peppermint oil, | | |
| | hydrogenated soybean oil, hydrogenated vegetable oils, Maisine | | |
| | 35-1 or Maisine CC or Peceol (glyceryl monolinoleate), Beeswax, | | |
| | oleic acid, soy fatty acids, Vitamin E, Labrafac, Miglyol, | | |
| | Crodamol, Capmuyl, Imwitor, Captex and other medium chain | | |
| | triglycerides (MCTs) | | |
| Co-solvent | Propylene Glycol, Capryol™ 90, Lauroglycol™ 90, Glycerin, | 0 | 15 |
| | Polyethylene Glycol | | |

### Dry Powder Blend Development

A dry powder blend may be compressed into a tablet or filled into a capsule. In one embodiment, the dry powder powder is compressed into a tablet for sublingual administration. The tablet may also be used for buccal administration. The tablet may also be swallowed (via oral administration). In another embodiment, a dry powder filled capsule may only be used for oral administration. Table 56 lists common excipients for dry powder which may be used for compression into tablets or filling capsules. Table 57 illustrates the formulation utilized for a tablet prototype batch A created by direct compression. All items were passed through a sieve screen and blended by appropriate means.

TABLE 57
Tablet Prototype Batch A Composition

| | | | Dose weight |
|---|---|---|---|
| Item # | Ingredient | % w/w | (mg) |
| 1 | Bumetanide Dibenzylamide | 15.0% | 30.0 |
| 2 | Ceolus KG 802 | 20.0% | 40.0 |
| | (Microcrystalline Cellulose) | | |
| 3 | Mannogem EZ (Spray Dried | 51.0% | 102.0 |
| | Mannitol) | | |
| 4 | Polyplasdone XL (Super | 7.0% | 14.0 |
| | Disintegrate) | | |
| 5 | Poloxamer 407 (Wetting | 3.0% | 6.0 |
| | Agent) | | |
| 6 | Citric Acid Monohydrate | 1.5% | 3.0 |
| 7 | Cabosil M5P (Fumed Silica) | 1.0% | 2.0 |
| 8 | Magnesium Stearate | 1.5% | 3.0 |
| | Total | 100.0% | 200.0 |

After successful completion of the dry blend tablet formulation, efforts were made to attempt to enhance solubilization of the drug. Upon successful creation of the tablet prototype batch A, the tablet prototype batch B was developed wherein Bumetanide Dibenzylamide solubilizers were incorporated directly into the dry blend tablet formulation. Visual solubility analysis provided knowledge that an approximate ratio of 1:2 Bumetanide Dibenzylamide to solubilizers were needed to dissolve the desired quantity of Bumetanide Dibenzylamide into 2 mL of water. The 1:1 ratio of Lauroyl Polyoxyl-32 Glycerides (Gelucire 44/14), to Sorbitan Ester (Span 80), was determined by combined melting point, taste, viscosity, and solubilizing ability. The ratio provided a semi-solid, which may be liquefied upon application of low heat. Utilizing a technique known as hot-melt granulation, the surfactants were liquefied at 60° C. followed by addition of the necessary quantity of Bumetanide Dibenzylamide and Poloxamer 407. This mixture was kept under constant stirring to ensure complete even dispersal of ingredients. Continuing under constant heating and mixing, Neusilin US2 (Magnesium Aluminometasilicate) was slowly added to adsorb the viscous mixture onto its particles rough surface. Upon complete adsorption the heat was removed with continued mixing until room temperature was reached. The cooled, hot-melt granulation was then sieved and blended with sieved Polyplasdone XL (Super Disintegrate), Cabosil M5P (fumed silica), and Citric Acid Monohydrate, and Magnesium Stearate. Table 58 illustrates the formulation employed for a sublingual tablet prototype batch B, which was utilized for canine studies.

TABLE 58
Sublinual Tablet Prototype Batch B Composition

| | | | Dose weight |
|---|---|---|---|
| Item # | Ingredient | % w/w | (mg) |
| 1 | Bumetanide Dibenzylamide | 7.4% | 30.0 |
| 2 | Gelucire 44/14 (Lauroyl Plyoxyl-32 | 9.9% | 40.0 |
| | Glycerides, a Water Dispersible | | |
| | Surfactant) | | |
| 3 | Span 80 (Sorbitan Ester, a Water | 9.9% | 40.0 |
| | Dispersible Surfactant) | | |
| 4 | Poloxamer 407 (Wetting Agent) | 0.5% | 0.8 |
| 5 | Neusilin US2 (Magnesium | 14.8% | 60.0 |
| | Aluminometasilicate) | | |
| 6 | Cabosil M5P (Fumed Silica) | 2.0% | 8.0 |
| 7 | Polyplasdone XL (Super Disintegrate) | 54.3% | 220.0 |
| 8 | Citric Acid Monohydrate | 0.7% | 3.0 |
| 9 | Magnesium Stearate | 0.5% | 2.0 |
| | Total | 100.0% | 405.0 |

A comparison of density analysis of both batches is provided in Tables 59-60. Visual monitoring of the tableting process revealed no weight fluctuations or die filling issues were present. Sublingual tablet prototype batch B flowdex score of 9 mm revealed that the flow of this composition is better than suggested that by the density analysis data. Contrasting this is the flowdex score of 26 mm recorded for the sublingual tablet prototype batch A, which indicates that the true dry blend flow may be worse than that suggested by the Carr's index or Hausner ratio. This issue regarding flowability may be remedied upon reduction of Bumetanide Dibenzylamide required for any human targeted dose strength (i.e., 10 mg dose).

TABLE 59
Dry Blend Physical Analysis
Sublingual Tablet Prototype Batch A

| Bulk Density | 0.344 g/mL | Tapped Density | 0.472 g/mL |
|---|---|---|---|
| Carr's Index | 27.0% | Hausner Ratio | 1.37 |

| | |
|---|---|
| Flowdex Score | 26 mm |

Prototype Batch B

| | | | |
|---|---|---|---|
| Bulk Density | 0.298 g/mL | Tapped Density | 0.355 g/mL |
| Carr's Index | 16.0% | Hausner Ratio | 1.19 |

| | |
|---|---|
| Flowdex Score | 9 mm |

TABLE 60
Dry Blend Physical Analysis Reference Table

| Flowability | Carr's Index (%) | Hausner Ratio |
|---|---|---|
| Excellent | 0-10 | 1.00-1.11 |
| Good | 10-15 | 1.12-1.18 |
| Fair | 16-20 | 1.19-1.25 |
| Slightly Poor | 21-25 | 1.26-1.34 |
| Poor | 26-31 | 1.35-1.45 |

### Sublingual Tableting Physical Comparisons

Due to their relatively small sizes, batches were not compressed by means of rotary tablet compression. Instead, a single station flexi-tab apparatus utilizing a feed shoe that fills a single die by means of gravity was employed. Tablets were purposely made soft with the understanding that packaging will ultimately be comprised of foil-to-foil blisters. Wetting refers to the time needed for blue dyed water to completely permeate through a tablet. Disintegration refers to the time needed for a tablet to be void of a hard core, determined by probing with a thin metal spatula. Table 61 lists the lists the physical properties of the two formulations.

TABLE 61
Comparison of Tablet Physical Parameters

| | | | | | |
|---|---|---|---|---|---|
| Tablet | Compression | Tablet | Tablet | Tablet | Wetting/ |
| Tooling | Force | Thickness | Weight | Hardness | Disintegration |

Sublingual Tablet Prototype Batch A

| | | | | | |
|---|---|---|---|---|---|
| 3/8” SC | 4 kN | 3.5 mm | 201 mg | 5.6 kP | 22 sec/32 sec |

Sublingual Tablet Prototype Batch B

| | | | | | |
|---|---|---|---|---|---|
| 1/2” SC | 3.5 kN | 5.2 mm | 410 mg | 3.2 kP | 29 sec/96 sec |

### Intermediate Stability

The two separate sublingual tablet formulations were monitored over a 2-week time period and monitored by visual appearance, assay, and related compound data across a 2 week 40° C./75% RH stability study. Specifics regarding tablet formulation composition, and physical characteristics may be referenced below. Both compositions consisted of tablets containing about 30 mg dosage strengths of Bumetanide Dibenzylamide designed to be delivered to the sublingual region. Tables and procedures listed outline the compositions and preparations utilized for the creation of both stability batches.

The items for preparation of the sublingual tablet prototype batch B composition are outlined in Table 58. Items #2-4 were heated in an appropriately sized vessel to 60° C. under constant stirring. Upon complete liquidation of items #2-4, item #1 was added to the vessel continuing constant temperature and mixing for 5-minutes. Item #5 was slowly added to the vessel with continuing constant temperature and mixing. Heat was removed and the granulation were mixed until items were reduced to room temperature. The granulation was sieved through a #40 mesh (420 micron) screen. Items #6-7 were sieved through a #40 mesh (420 micron) screen. Item #8 was sieved through a #20 mesh (840 micron) screen. The granulation and items #6-8 were added to an appropriately sized vessel. The vessel was about 60% charged with material. A 3D shake mixer (turbula) was used to blend items #1-8 for about 20-minutes. Item #9 was sieved through a #40 mesh (420 micron) screen. Item #9 was added to the vessel containing items #1-8 and blended by 3D shake mixer for about 5-minutes. 405 mg tablets were created using a ½″ standard concave tooling with 3.5 kN of force.

The items for preparation of the sublingual tablet prototype batch A composition are outlined in Table 57. Items #1-5 were sieved through a #40 mesh (420 micron) screen. Item 6 was sieved through a #20 mesh (840 micron) screen. Items 1-6 were added to an appropriately sized vessel. The vessel was about 60% charged with material. A 3D shake mixer (turbula) was used to blend items #1-6 for about 20-minutes. Item #7 was sieved through a #40 mesh (420 micron) screen. Item #7 was added to the vessel containing items #1-6, and blended by a 3D shake mixer for 5-minutes. Item #8 was sieved through a #40 mesh (420 micron) screen. Item #8 was added to the vessel containing items #1-7 and blended by 3D shake mixer for about 5-minutes. 200 mg tablets were created using ⅜″ standard concave tooling with 4 kN of force.

### Stability Storage and Analysis

The study was conducted with tablets stored at 40° C./75% RH. Tablets were stored at 30 count per bottle complete with coil, and a 1 gram desiccant in heat sealed 60 cc HDPE bottles. Tablets were monitored for their respective physical appearance, assay values, and related compounds.

Samples were stored and tested as per Table 62. Analysis was done at time zero (T=0) and at 2 weeks, wherein the compositions were kept at 40° C./75% RH. The target criteria were 90.0-110.0% of LC.

TABLE 62
Stability Testing Criteria

| | Time | | |
|---|---|---|---|
| | Point | | |
| | (weeks) | | |
| Test | T − 0 | 2 wk | Target Criteria |
| Assay of Bumetanide | X | A | 90.0-110.0% of LC |
| Dibenzylamide | | | |
| Related Compounds | X | A | Report Results > |
| | | | 0.05% |
| Appearance | X | A | Report Results |

X: Time Zero
A: 40° C./75% RH

The results were recorded over the 2-week stability period monitored. Analysis was broken down by sections into Visual Appearance Results, Assay Results, and Related Compounds Results. All tablets conformed to the appearance specification and there was no significant change in the appearance of the tablets for each batch over the first 2 weeks of the stability study. The appearance results are presented in Table 63. All time points analyzed were well within stability criteria (Tables 64). Assay values appear to have decreased over the stability period. However, due to the lack of increase in related compounds over the same time it may be concluded that the drop in assay values are extremely unlikely to have been caused by degradation. It is important to note that due to the minimal time points analyzed a trend cannot be established.

TABLE 63
Appearance Results for Stability Batches

| | Time | | |
|---|---|---|---|
| Batch | Point | Appearance Specification | Results |
| Batch A | Initial | Off-white tablet with no | Conforms |
| 40° C./ | 2 Weeks | visible erosion, discoloration, | Conforms |
| 75% RH | | or defects on the surface | |
| Batch B | Initial | Off-white tablet with no | Conforms |
| 40° C./ | 2 Weeks | visible erosion, discoloration, | Conforms |
| 75% RH | | or defects on the surface | |

TABLE 64
Sublingual Tablet Assay Stability Results

| Formulation | Time Point | Assay |
|---|---|---|
| Secondary Prototype | T = 0 weeks | 99.4 |
| Batch#184A-2010 | T = 2 weeks | 94.2 |
| Primary Prototype | T = 0 weeks | 107.7 |
| Batch#184A-2009 | T = 2 weeks | 101.3 |

Neither batch monitored over the 2-week stability period accrued any notable increases to related compounds (Table 65). The only related compounds recorded had negligible change over the time analyzed. This provides evidence that all processing methods utilized between both batches had little effect on the chemical stability of the Bumetanide Dibenzylamide, providing confidence that a hot melt granulation technique may be performed without extreme concern of degradation. Thermodynamic results gathered form DSC analysis provides further confidence that temperature exposure up to 60° C. is tolerable.

TABLE 65
Related Compound Results for Stability Batches

| | | Total % | | |
|---|---|---|---|---|
| | | Area | Related | Related |
| | Time | Related | Compound | Compound |
| Formulation | Point | Compound | @ RRT0.803 | @ RRT0.861 |
| Secondary | T = 0 weeks | 0.12 | 0.08 | 0.04 |
| Prototype | T = 2 weeks | 0.13 | 0.09 | 0.04 |
| Batch#184A-2010 | | | | |
| Primary Prototype | T = 0 weeks | 0.14 | 0.09 | 0.05 |
| Batch#184A-2009 | T = 2 weeks | 0.13 | 0.09 | 0.04 |

Results confirm both formulations analyzed to be stable and acceptable for animal studies within the time-period monitored. The hot melt granulation did not completely solubilize all the Bumetanide Dibenzylamide, instead forming a slurry or suspension. The slurry would have allowed for a multitude of these agglomerates to persist throughout processing, similar to a direct blend technique.

Remedies to these issues include increasing batch size and milling to obtain a more uniform particle size. Specific to the hot melt granulation procedure, additional surfactants, and solubilizers may be added to completely solubilize the Bumetanide Dibenzylamide. This may result in additional dry substrate to sorb the increased liquid/semi-solid concentration resulting in larger tablets that are suitable for oral administration. The maximum amount of bumetanide dibenzylamide in the dry powder formulation is based on the solubility in the solubilizer reduced by the amount of absorbent and about half the total weight of the dosage form. The sublingual tablet composition B Table 54 utilizes a 1:1 mixture of Gelucire 44/14 and Span 80 as solubilizer and Neusilin US2 absorbent. Since the solubility of bumetanide dibenzylamide in the 1:1 mixture was determined to be approximately 500 mg/g and the amount of Neusilin US2 absorbent required was about 15%, the maximum dose for the sublingual tablet composition B Table 54 is up to about 70 mg or about 17.5%. For sublingual administration the tablet size should be minimized but larger tablets and capsules up to about 1 gram are acceptable for oral administration. For oral administration the tablet or capsule may be formulated up to about 175 mg (17.5%) per dose. However, due to the known 3× decrease in dosage strength which will accompany human needs such an increase in excipients may still likely result in a tablet formulation below about 250 mg per dose.

### Example 7

### Injection Formulation for SC, IM, IV Bolus, IV Infusion

The excipients listed in Table 66 are proposed based on the functional category and prior history of use. The ranges provided are estimates based on the available data obtained during the initial development, prior experience and literature research. These ranges represent the space in which a suitable solution for injection may be obtained in combination with the other excipients in ratios adjusted to total 100% w/w.

TABLE 66
Common excipients for injection

| | | Range | |
|---|---|---|---|
| Class/Uses | Excipient examples | (% w/w) | |
| Non-ionic | Polyoxyl 35 Castor Oil (Kolliphor EL) or | 0 | 65 |
| surfactant/ | Cremophor RH40 (polyoxyl castor oil), | | |
| solubilizer, | Tween 80 (polysorbate), Solutol HS-15 | | |
| emulsifying | | | |
| agent | | | |
| Water - | soybean oil, castor oil, sesame oil, cotton | 0 | 100 |
| insoluble | seed oil, safflower seed oil, olive oil, | | |
| Solvent | coconut oil, Vitamin E, cholesterol, | | |
| | medium chain triglycerides (MCTs) | | |
| Water soluble | Propylene Glycol, Glycerin, Polyethylene | 0 | 90 |
| Solvent | Glycol 300/400, ethanol, N-Methyl | | |
| | pyrrolidone, dimethylacetamide, dimethyl | | |
| | sulfoxide, benzylalcohol | | |
| Other | Hydroxypropyl-b-cyclodextrin, | 0 | 30 |
| solubilization | sulfobutylether-b-cyclodextrin, a- | | |
| agent | cyclodextrin, | | |
| Phospholipids | Hydrogenated soy phosphatidylcholine, | 0 | 20 |
| | distearoylphosphatidylglycerol, L-a- | | |
| | dimyristoylphosphatidylcholine, L-a- | | |
| | dimyristoylphosphatidylglycerol, Lecithin, | | |
| | Phosal75A | | |

### Sample Preparation

A study was conducted to evaluate the solubility of Bumetanide Dibenzylamide in various solvent/co-solvent systems. Multiple vehicle compositions were investigated, resulting in a clear dosing solution or suspension at the time of the preparation or shortly after the preparation. Vehicles tested included 55% Polyethylene glycol 400 (PEG-400) in water, 41% PEG-400, 12% Ethanol, 47% water, 45% PEG-400, 10% DMSO, 45% Water, 31% PEG-400, 31% Tetraglycol, 15% Caprylocaproyl Polyoxylglycerides (Labrasol ALF), 23% water, 30% PEG-400, 10% N-Methyl Pyrrolidone, 10% DMSO, 50% water, 25% PEG-400, 10% DMSO, 20% Tetraglycol, 45% water, 20% Ethanol in water, 20% Hydroxypropyl-β-Cyclodextrin in water, 11% DMSO, 22% PEG-400, 22% Tetraglycol, 44% water, 44% PEG-400, 0.4% Poloxamer-188, 22% N-Methyl Pyrrolidone, 33% water, and 20% PEG-400, 15% Hydroxypropyl-β-Cyclodextrin in water.

Multiple vehicle compositions were tested, however all compositions tested resulted in a suspension sample either at the time of the preparation or shortly after the preparation. The final concentration of bumetanide dibenzylamide obtained for the above-mentioned vehicles was calculated at the point of precipitation ranged between 1.25 to 5.56 mg/mL. Unexpectedly, a vehicle composition of 40% PEG-400, 20% Propylene glycol, 5% Ethanol, 35%, water resulted in a clear solution for a period of at least about 3 hours at room temperature. After filtration with a 0.22 μm PVDF filter, the sample stayed clear for up to 7 days. The compositions selected for animal studies are described in Table 67 and Table 68.

TABLE 67
Injection 2.5 mg/ml

| Item # | Ingredient | % w/w | % w/v | mg/mL |
|---|---|---|---|---|
| 1 | Bumetanide | 0.23 | 0.25 | 2.5 |
| | dibenzylamide | | | |
| 2 | Propylene Glycol | 20 | 22 | 220 |
| 3 | Polyethylene | 40 | 44 | 440 |
| | Glycol 400 | | | |
| 4 | Ethanol | 5 | 5.5 | 55 |
| 5 | Water | 35 | 38.5 | 385 |
| | Total | 100.00 | 111.00 | 1100.00 |

TABLE 68
Injection 30 mg/mL

| Item# | Ingredient | % w/w | % w/v | mg/mL |
|---|---|---|---|---|
| 1 | Bumetanide | 2.73 | 3.00 | 30.00 |
| | dibenzylamide | | | |
| 2 | Caprylocaproyl | 10.00 | 11.00 | 110.00 |
| | Polyoxylglycerides | | | |
| | (Labrasol ALF) | | | |
| 3 | Propylene Glycol | 12.05 | 13.26 | 132.60 |
| 4 | Polyethylene Glycol | 67.22 | 73.94 | 739.40 |
| | 400 | | | |
| 5 | Water | 8.00 | 8.80 | 88.00 |
| | Total | 100.00 | 110.00 | 1100.00 |

For injection compositions, Bumetanide Dibenzylamide concentration may be adjusted from about 20 mg/mL to about 40 mg/mL (2 to 4%) with corresponding range of water content 8 to 20% maintaining ratio of all other excipients for a total weight of 100%. For injection compositions with >20% water content, Bumetanide dibenzylamide concentration is limited to up to about 2.5 mg/mL (0.25%).

### Example 8: Oral Liquid (Solution and Suspension)

The excipients listed in Table 69 are proposed based on the functional category and prior history of use. The ranges provided are estimates based on the available data obtained during the initial development, prior experience, and literature research. These ranges represent the space in which a suitable solution or suspension for oral delivery may be obtained in combination with the other excipients in ratios adjusted to total 100% w/w.

TABLE 69
Common excipients for oral solution and suspension

| | | Range | |
|---|---|---|---|
| Class/Uses | Excipient examples | (% w/w) | |
| Non-ionic | Caprylocaproyl polyoxylglycerides (Labrasol ALF and Sopftigen), Lauroyl | 0 | 80 |
| surfactant/ | polyoxylglycerides (Gelucire 44/14 or 48/16), Linoleoyl polyoxylglycerides | | |
| solubilizer, | (Labrafil M2125CS), Oleoyl polyoxylglycerides (Labrafil M1944CS), | | |
| emulsifying | Stearoyl polyoxylglycerides (Gelucire 50/13), Polyoxyl 35 Castor Oil | | |
| agent | (Kolliphor EL) or Cremophor RH40 (polyoxyl castor oil), vitamin E | | |
| | TPGS, Span 20 (Sorbitan oleate), Tween 20 or 80 (polysorbate), | | |
| | Poloxamers, Solutol HS-15, | | |
| Water- | Arachis oil, soybean oil, castor oil, corn oil, safflower oil, olive oil, apricot | 0 | 80 |
| insoluble | kernel oil, sesame oil, cotton seed oil, sunflower seed oil, palm seed oil, | | |
| Solvent | coconut oil, rapeseed oil, peanut oil, peppermint oil, hydrogenated soybean | | |
| | oil, hydrogenated vegetable oils, Maisine 35-1 or Maisine CC (glyceryl | | |
| | monolinoleate), Beeswax, oleic acid, soy fatty acids, Vitamin E, Labrafac, | | |
| | Miglyol, Crodamol, Capmuyl, Imwitor, Captex and other medium chain | | |
| | triglycerides (MCTs) | | |
| Water | Propylene Glycol, Capryol ™ 90, Lauroglycol ™ 90, Glycerin, | 0 | 90 |
| soluble | Polyethylene Glycol 300/400/600, ethanol, N-Methyl pyrrolidone, | | |
| Solvent | tetraglycol, water, dimethylacetamide, dimethyl sulfoxide | | |
| Other | Hydroxypropyl-b-cyclodextrin, sulfobutylether-b-cyclodextrin, a- | | |
| solubilization | cyclodextrin | | |
| agent | | | |
| Phospho- | Hydrogenated soy phosphatidylcholine, distearoylphosphatidylglycerol, | | |
| lipids | L-a-dimyristoylphosphatidylcholine, L-a-dimyristoylphosphatidylglycerol | | |
| Thickening | Carbomer, Pemulen (synthetic polymers of acrylic acid) Hydroxypropyl | 0 | 10 |
| agent | cellulose, carboxymethylcellulose, hydroxyethyl cellulose, ethylcellulose, | | |
| | xylulose (cellulose based polymers) | | |
| | Xanthan gum, gellan gum, guar gum, pectin, gelatin, starch, alginate, | | |
| | tragacanth, bentonite, chitosan, carrageenan, acacia (natural polymers) | | |
| | Polyvinyl alcohol, polyethylene oxide, Poloxamer or Pluronic, polyvinyl | | |
| | pyrrolidone or povidone, Polyethylene glycol 3350 (synthetic polymers) | | |

### Sample Preparation

Sample was prepared as previously described under sample development for injections under Example 7. In addition, the compositions shown in Table 47 and Table 48 (used for nasal administration) are also oral solution formulations for bumetanide dibenzylamide.

The solution compositions selected for animal studies are described in Table 70 and Table 71.

TABLE 70
Oral Solution 2.5 mg/mL

| Item# | Ingredient | % w/w | % w/v | mg/ml |
|---|---|---|---|---|
| 1 | Bumetanide | 0.23 | 0.25 | 2.5 |
| | dibenzylamide | | | |
| 2 | Propylene Glycol | 20 | 22 | 220 |
| 3 | Polyethylene Glycol | 40 | 44 | 440 |
| | 400 | | | |
| 4 | Ethanol | 5 | 5.5 | 55 |
| 5 | Water | 35 | 38.5 | 385 |
| | Total | 100.00 | 111.00 | 1100.00 |

TABLE 71
Oral Solution 30 mg/mL

| Item# | Ingredient | % w/w | % w/v | mg/ml |
|---|---|---|---|---|
| 1 | Bumetanide | 2.82 | 3.13 | 31.27 |
| | Dibenzylamide | | | |
| 2 | Caprylocaproyl | 4.65 | 5.17 | 51.67 |
| | Polyoxylglycerides | | | |
| | (Labrasol ALF) | | | |
| 3 | Propylene Glycol | 13.01 | 14.44 | 144.44 |
| 4 | Polyethylene Glycol 400 | 67.97 | 75.45 | 754.49 |
| 5 | Polyvinylpyrrolidone | 0.92 | 1.03 | 10.25 |
| | (K30) | | | |
| 6 | Water | 10.62 | 11.79 | 117.89 |
| | Total | 100.00 | 111.00 | 1110.00 |

TABLE 72
Oral Suspension 20 mg/mL

| Item# | Ingredient | % w/w | % w/v | mg/ml |
|---|---|---|---|---|
| 1 | Bumetanide | 1.82 | 2 | 20 |
| | Dibenzylamide | | | |
| 2 | Carboxymethylcellulose | 0.45 | 0.5 | 5 |
| | (medium viscosity) | | | |
| 3 | Water | 97.7 | 107.5 | 117.89 |
| | Total | 100.00 | 111.00 | 1110.00 |

For oral solution compositions, Bumetanide dibenzylamide concentration may be adjusted from about 20 mg/mL to about 40 mg/mL (2 to 4%) with corresponding range of water content 8 to 20% maintaining ratio of all other excipients for a total weight of 100%. For compositions with >20% water content, Bumetanide dibenzylamide concentration is limited to up to about 2.5 mg/mL (0.25%). For oral suspension compositions, Bumetanide dibenzylamide concentration may be adjusted up to about 40 mg/mL (4%) with the addition of a wetting agent or surfactant. One embodiment of an oral suspension composition selected for animal studies is shown in Table 72. A suspension intended for oral gavage administration) was 0.5% w/v Carboxymethylcellulose (medium viscosity) in purified water.

### Example 9: Permeability of Test Compounds in Caco-2 Assay

Compounds of the present disclosure were tested in a gold standard permeability assay. Unexpectedly, the results demonstrate the tested compounds would not be expected to provide improved permeability. Table 73 demonstrates the unpredictable nature of drug product development.

TABLE 73
Permeability Assay for Different Compounds

| | | | | Mean % Solution | | Rank | |
|---|---|---|---|---|---|---|---|
| | Mean Pₐₚₚ (10⁻⁶ cm/s) | | Efflux | Recovery | | | Efflux Transporter |
| Compound | A to B | B to A | Ratio | A to B | B to A | Pₐₚₚ | Substrate |
| Nadolol | 0.0983 | ND | — | 97.0 | ND | Low | — |
| Metoprolol | 19.2 | ND | — | 94.7 | ND | High | — |
| Digoxin | 0.0223 | 13.1 | 586 | 94.7 | 108.1 | Low | Likely |
| Bumetanide | <0.480 | 0.396 | >0.825 | <4.9 | 25.4 | Low | Poor or non- |
| Dibenzylamide | | | | | | | |
| unformulated | | | | | | | |
| Bumetanide | 0.709 | 31.0 | 43.8 | 87.4 | 99.6 | Moderate | Likely |
| morpholinoamide | | | | | | | |
| unformulated | | | | | | | |
| Bumetanide | <0.332 | 0.199 | >0.600 | <58.3 | 86.3 | Low | Poor or non- |
| Dibenzylamide | | | | | | | |
| SEDDS | | | | | | | |
| Bumetanide | 0.787 | 33.8 | 42.9 | 98.2 | 102.2 | Moderate | Likely |
| morpholinoamide | | | | | | | |
| SEDDS | | | | | | | |

TABLE 73
Permeability Assay for Different Compounds

| | | | | | | Rank | |
|---|---|---|---|---|---|---|---|
| | | | | Mean % Solution | | | Efflux |
| | Mean Pₐₚₚ (10⁻⁶ cm/s) | | Efflux | Recovery | | | Transporter |
| Compound | A to B | B to A | Ratio | A to B | B to A | Pₐₚₚ | Substrate |
| Nadolol | 0.0518 | ND | — | 98.5 | ND | Low | — |
| Metoprolol | 15.5 | ND | — | 95.7 | ND | High | — |
| Digoxin | 0.0154 | 12.3 | 800 | 97.4 | 103.4 | Low | Likely |
| Bumetanide | 1.33 | 42.0 | 31.6 | 77.4 | 88.5 | Moderate | Likely |
| Diethylamide | | | | | | | |
| Bumetanide | 1.48 | 32.0 | 21.6 | 79.3 | 90.2 | Moderate | Likely |
| Diethylamde | | | | | | | |
| SEDDS | | | | | | | |

The signal responses of Bumetanide Dibenzylamide in A to B receiver samples were undetectable. The signal responses of Bumetanide Dibenzylamide SEDDS in one A to B receiver sample was undetectable. For the convenience of calculation, 1/300 of the measured peak area ratio (PAR) of the relevant TO samples were used as the PAR values in these receiver samples. As will be appreciated, using an assumption of zero as the peak area in above conditions, the solution recovery of Bumetanide Dibenzylamide SEDDS was greater than the acceptable cut-off value of 50.0, so the recovery of Bumetanide Dibenzylamide SEDDS was sufficient. The insufficient recovery (% Solution Recovery values <50.0) observed might complicate the results of Bumetanide Dibenzylamide, therefore, the results generated in this study should be interpreted combining with other in vitro and/or in vivo data as presented herein. The efflux phenomena (efflux ratio >2.00) observed might complicate the prediction of permeability. Therefore, the permeability results of Bumetanide Morpholinoamide and Bumetanide Morpholinoamide SEDDS generated in this study should be interpreted in combination with the application conditions. The efflux phenomena (efflux ratio >2.00) observed might complicate the prediction of permeability. Therefore, the permeability results of Bumetanide Diethylamide and Bumetanide Diethylamide SEDDS generated in this study should be interpreted in combination with the application conditions. ND means not determined. Binning Criteria: Low permeability: Pₐₚₚ≤0.500 (×10⁻⁶ cm/s); Moderate permeability: 0.500<Pₐₚₚ<2.50 (×10⁻⁶ cm/s); High permeability: Pₐₚₚ≥2.50 (×10⁻⁶ cm/s); and *The binning criteria of permeability are proposed based on Creative Bioarray routine Caco-2 permeability assay conditions (2.00 UM dosing concentration and 120 minutes incubation). The boundaries for low and high permeability binning are equivalent to 50% and 80% of the “calculated Fa” in human; Substrate Potential **; likely: ERa≥2.00; Poor or non: ERa<2.00.

### Methodology

### Caco-2 Culture

Caco-2 cells purchased from ATCC were seeded onto polyethylene membranes (PET) in 96-well Corning Insert plates at 1×10⁵ cells/cm², and refreshed medium every 4˜5 days until to the 21^{st} to 28^{th} day for confluent cell monolayer formation. Table 74 lists the compound information.

TABLE 74
Compound Information

| Serial Number | Compound ID FW | Batch No | MW |
|---|---|---|---|
| CPD1 | Bumetanide | 60495-25-34 | 543.68 |
| | Dibenzylamide | | |
| | 543.68 | | |
| CPD2 | Bumetanide | 60646-07-34 | 433.53 |
| | Dibenzylamide | | |
| | 433.53 | | |
| CPD3 | Bumetanide | 32920-03-28 | 543.68 |
| | Dibenzylamide | | |
| | SEDDS | | |
| | 543.68 | | |
| CPD4 | Bumetanide | 32920-04-16 | 433.53 |
| | Dibenzylamide | | |
| | SEDDS | | |
| | 433.53 | | |

TABLE 74′
Compound Information

| Serial Number | Compound ID | Batch No | MW |
|---|---|---|---|
| CPD1 | Bumetanide | 60646-09-18 | 419.54 |
| | Diethylamde | | |
| CPD2 | Bumetanide | 32920-06-04 | 419.54 |
| | Diethylamde | | |
| | SEDDS | | |

### Transport-Methods

The transport buffer in the study was HBSS with 10.0 mM HEPES at pH 7.40±0.05. Test compound was tested at 2.00 μM bi-directionally in duplicate. Digoxin was tested at 10.0 μM bi-directionally in duplicate, while nadolol and metoprolol were tested at 2.00 μM in A to B direction in duplicate. Final DMSO concentration was adjusted to less than 1%. The plate was incubated for 2 hours in CO₂ incubator at 37±1° C., with 5% CO₂ at saturated humidity without shaking. And all samples after mixed with acetonitrile containing internal standard were centrifuged at 3200×g for 10 min. For nadolol and metoprolol, 200 μL supernatant solution was diluted with 600 μL ultra-pure water for LC-MS/MS analysis. For digoxin and test compounds, 200 μL supernatant solution was diluted with 200 μL ultra-pure water for LC-MS/MS analysis. Concentrations of test and control compounds in starting solution, donor solution, and receiver solution were quantified by LC-MS/MS methodologies, using peak area ratio of analyte/internal standard. After transport assay, lucifer yellow rejection assay was applied to determine the Caco-2 cell monolayer integrity.

### Data Analysis

The apparent permeability coefficient Pₐₚₚ (cm/s) was calculated using the equation:

$${P}_{app} = ( d {C}_{r} / d t ) \times {V}_{r} / ( A \times {C}_{0} )$$

Where dCₚ/dt is the cumulative concentration of compound in the receiver chamber as a function of time (μM/s); Vᵣ is the solution volume in the receiver chamber (0.075 mL on the apical side, 0.25 mL on the basolateral side); A is the surface area for the transport, i.e. 0.0804 cm² for the area of the monolayer; Co is the initial concentration in the donor chamber (μM). The efflux ratio was calculated using the equation:

$$Efflux Ratio = {P}_{app} ( B A ) / {P}_{app} ( AB )$$

Percent recovery was calculated using the equation:

$$\% Solution Recovery = 100 \times [ ( {V}_{r} \times {C}_{r} ) + ( {V}_{d} \times {C}_{d} ) ] / ( {V}_{d} \times {C}_{0} )$$

Where V_{d} is the volume in the donor chambers (0.075 mL on the apical side, 0.25 mL on the basolateral side); Ca and Cr are the final concentrations of transport compound in donor and receiver chambers, respectively.

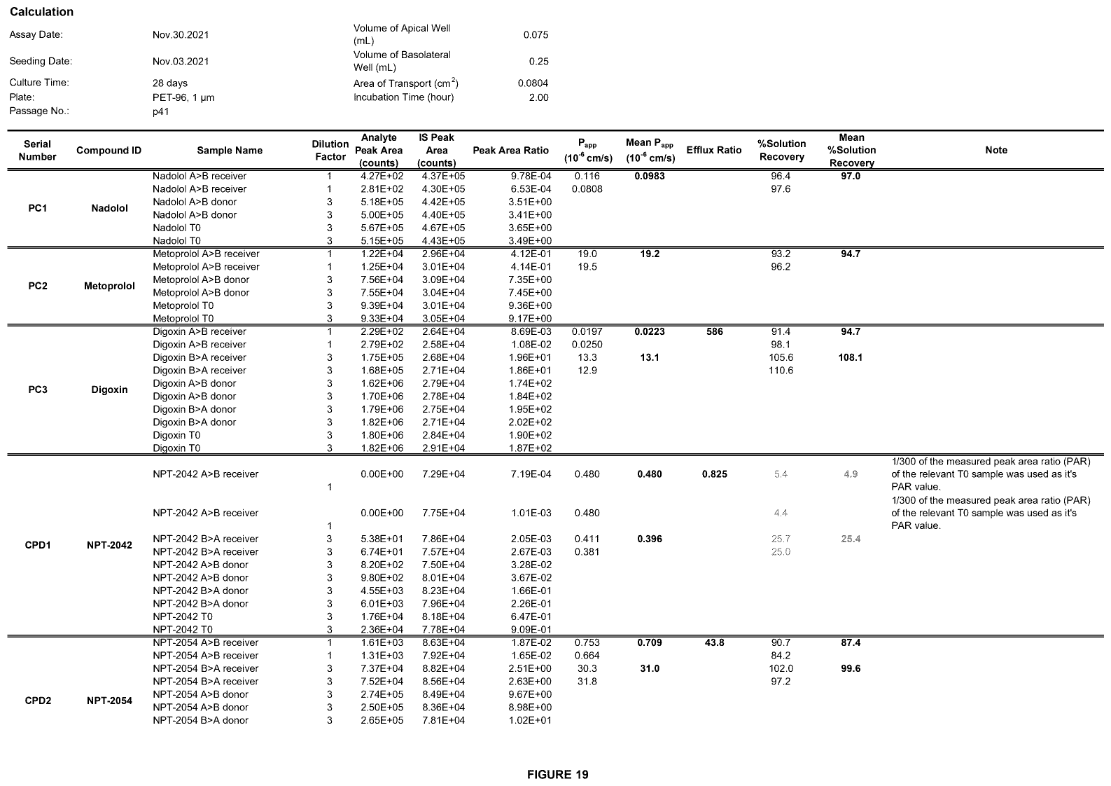

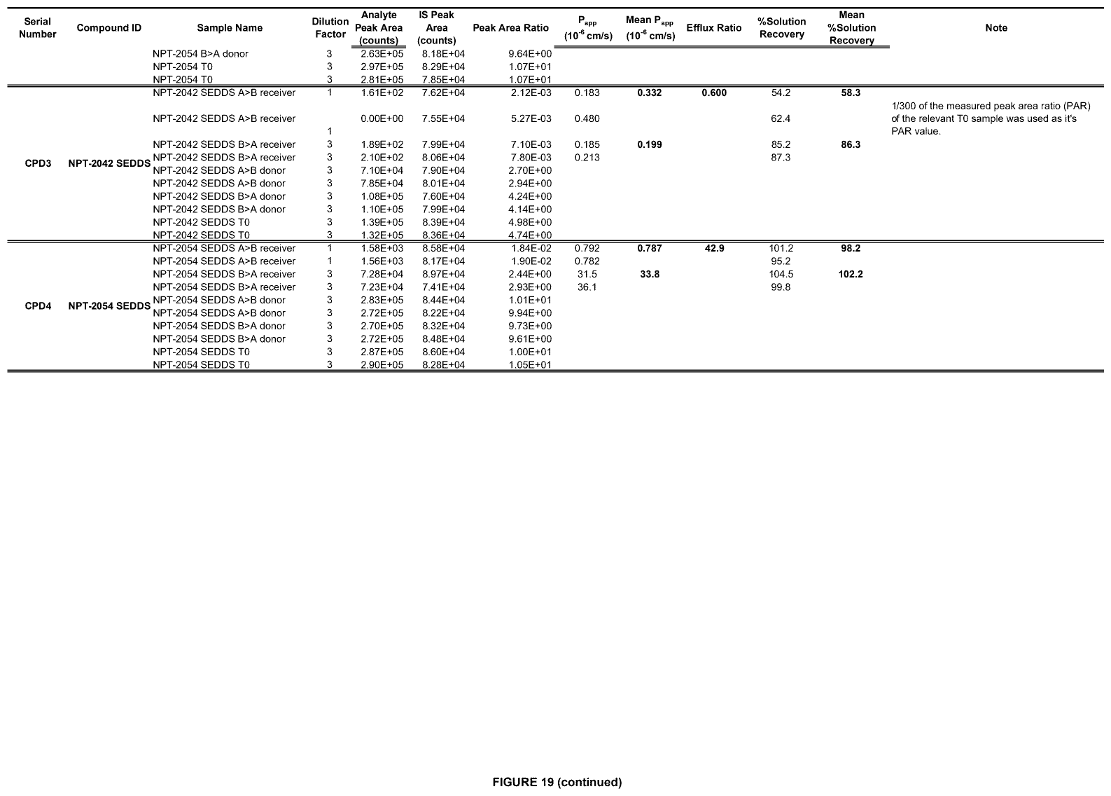

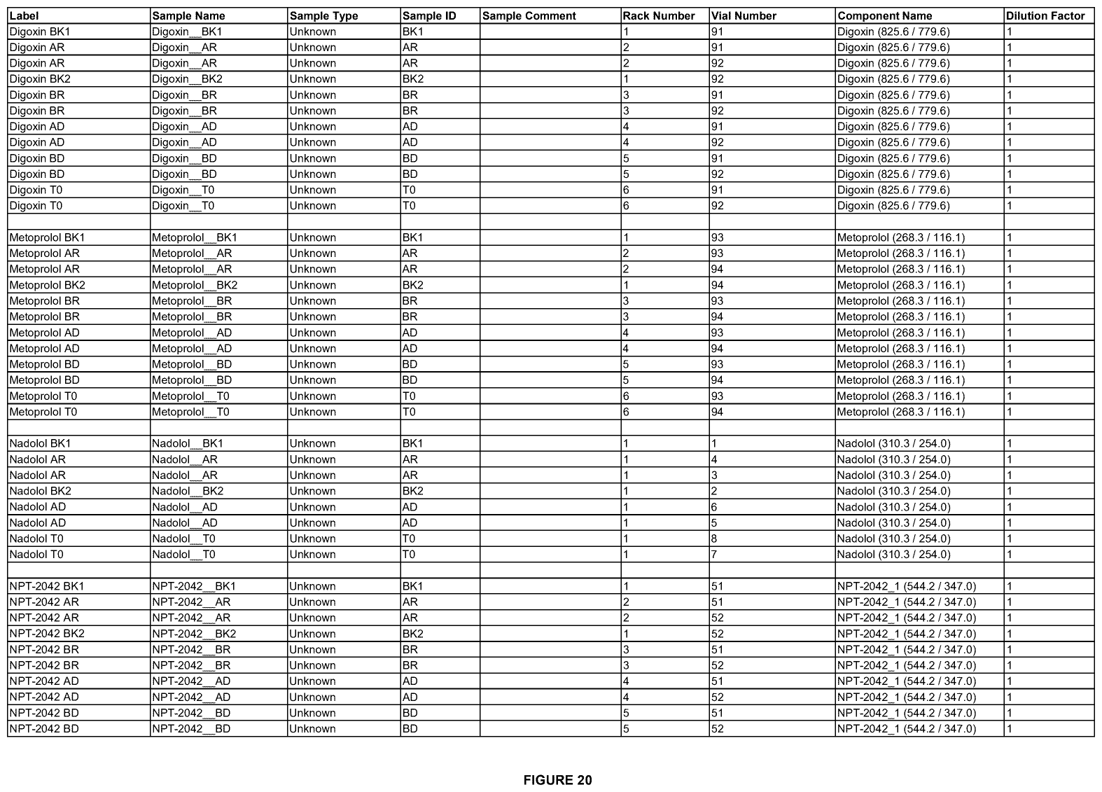

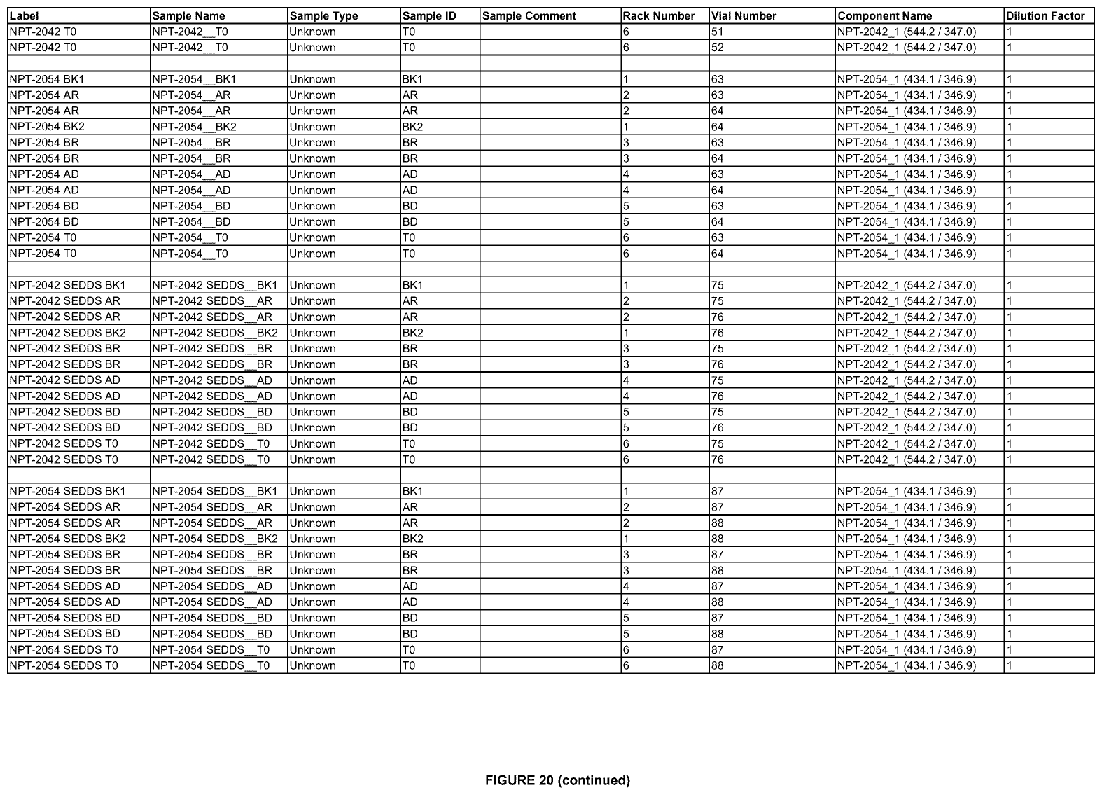

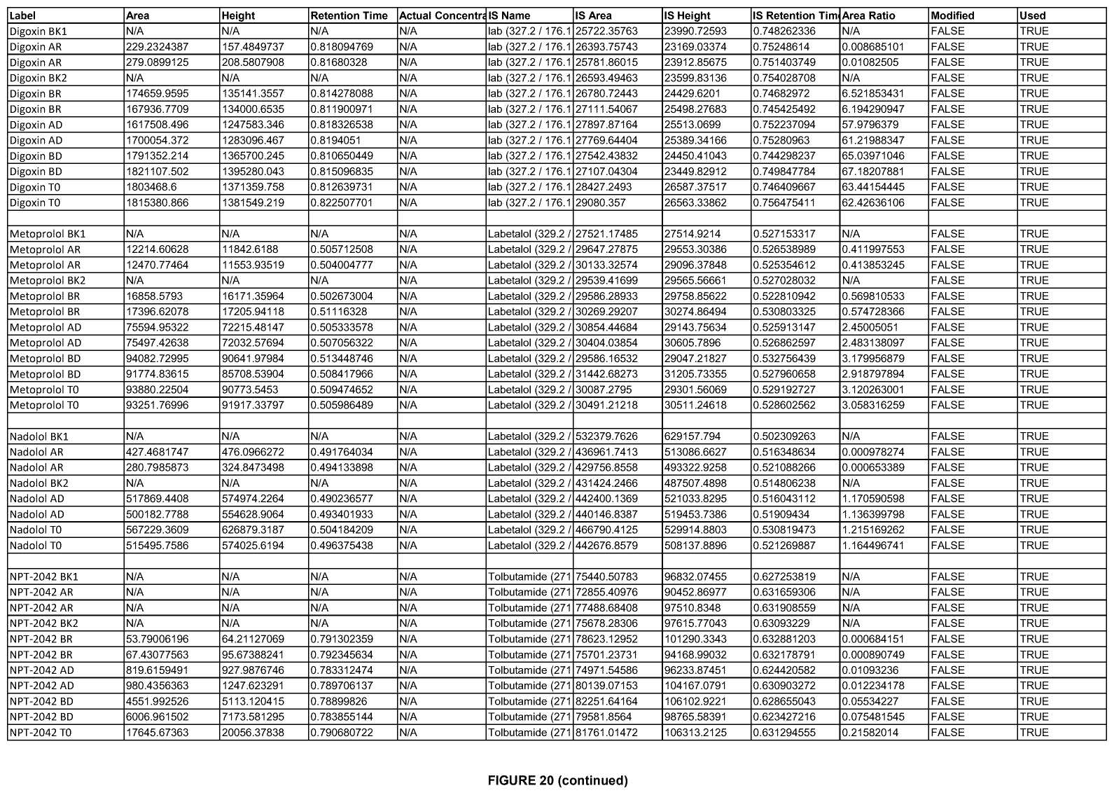

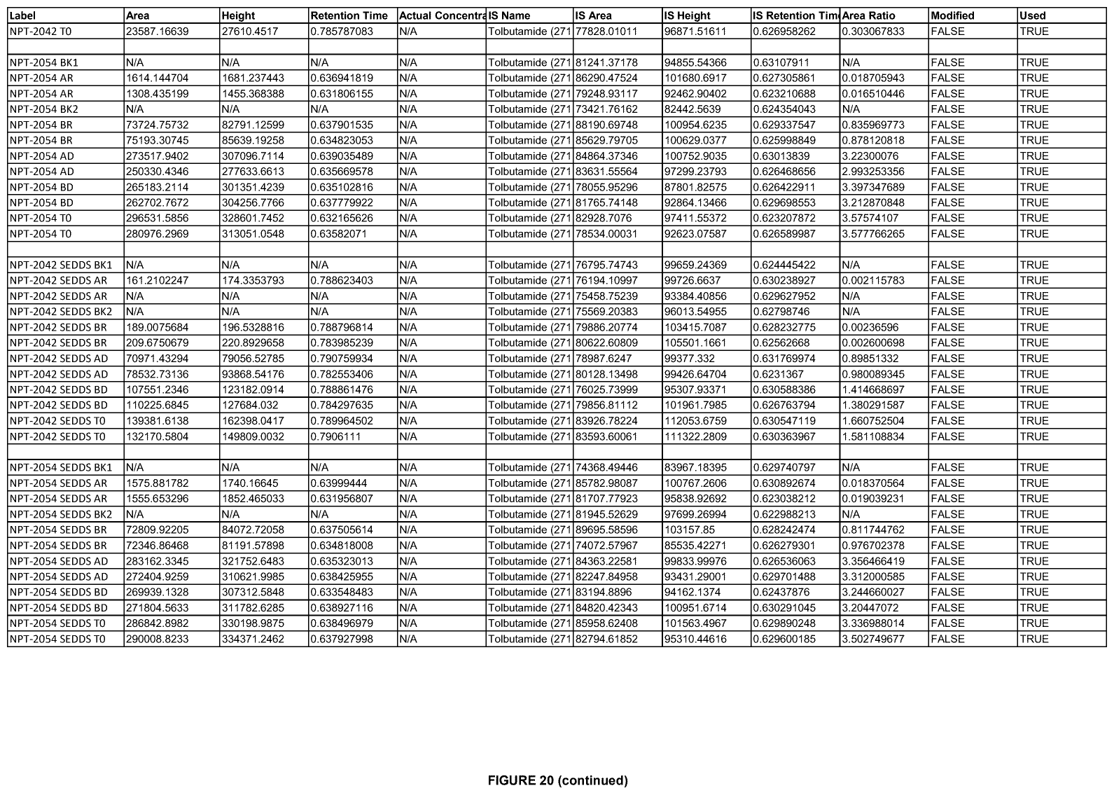

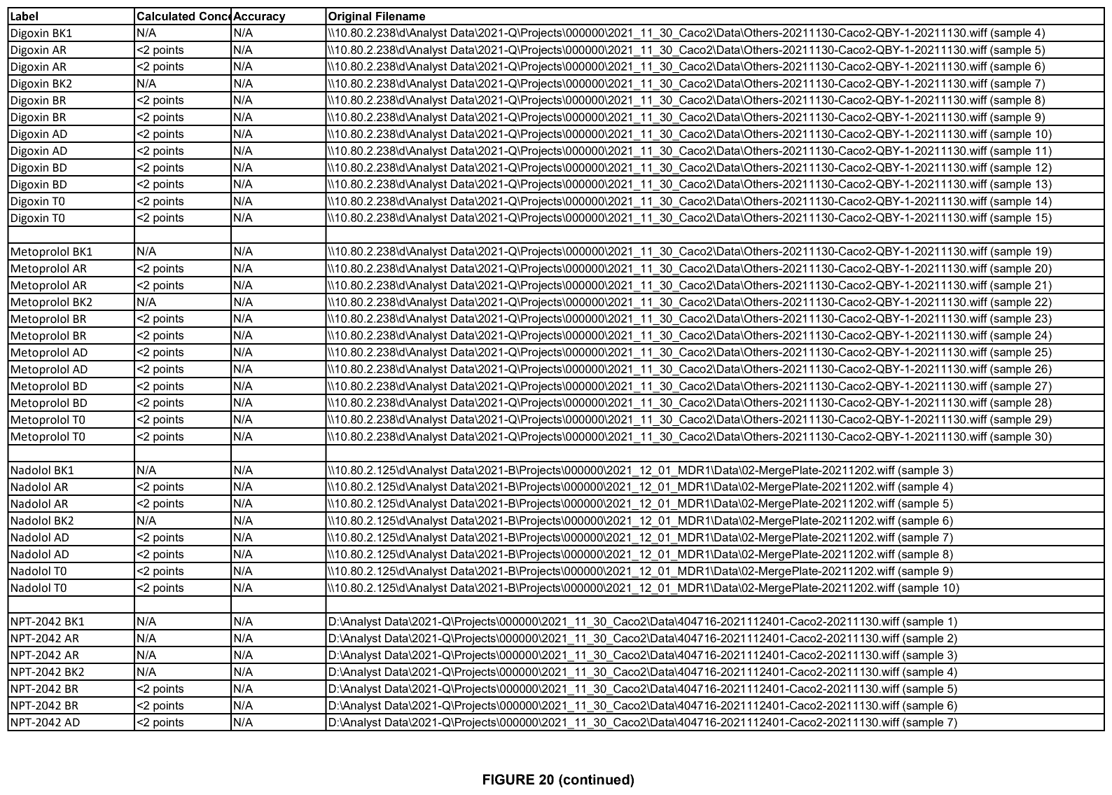

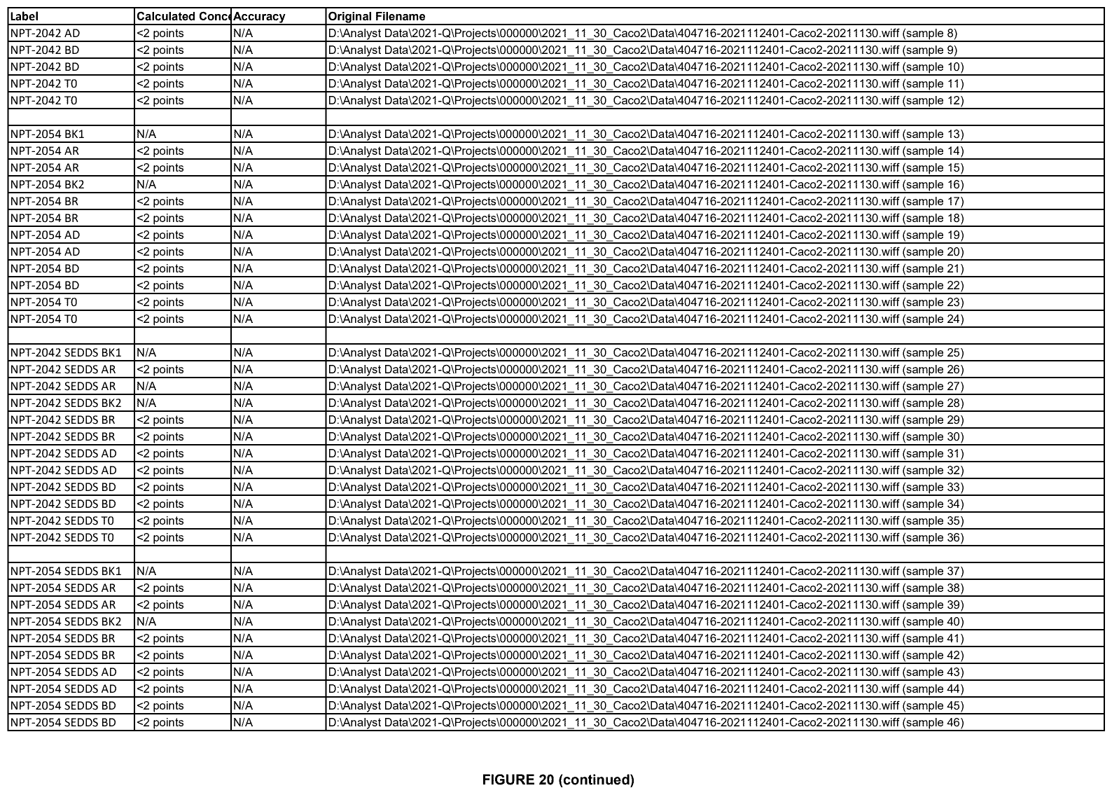

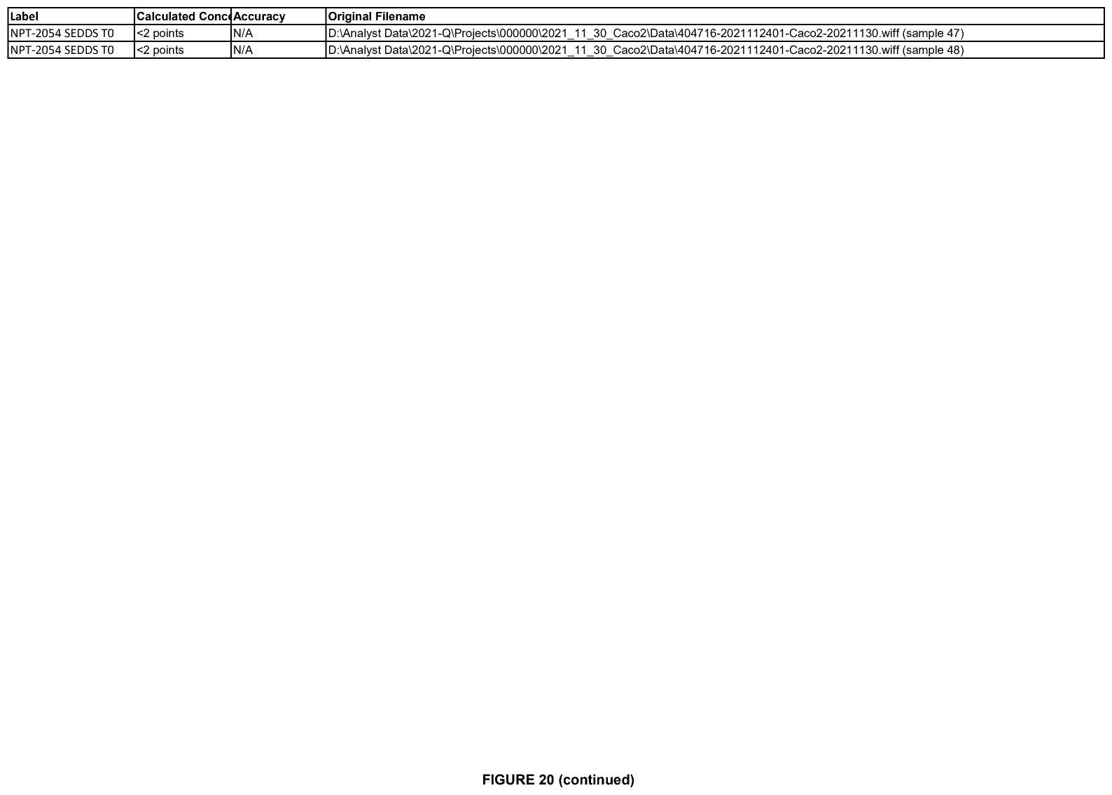

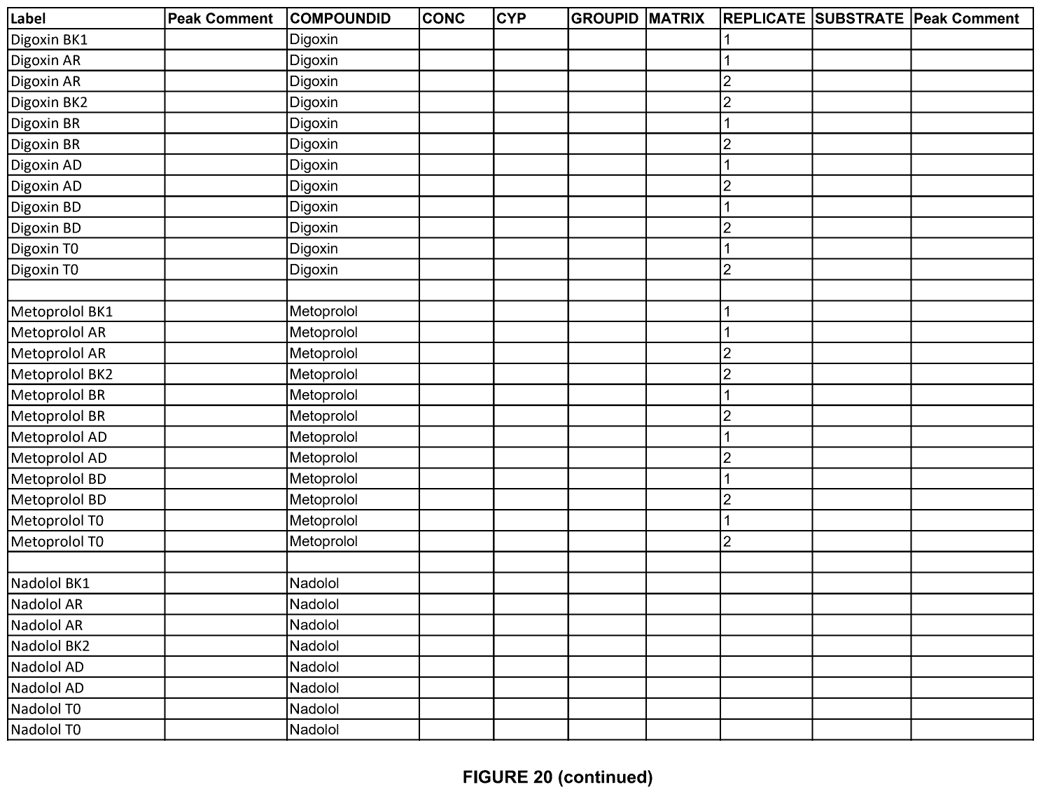

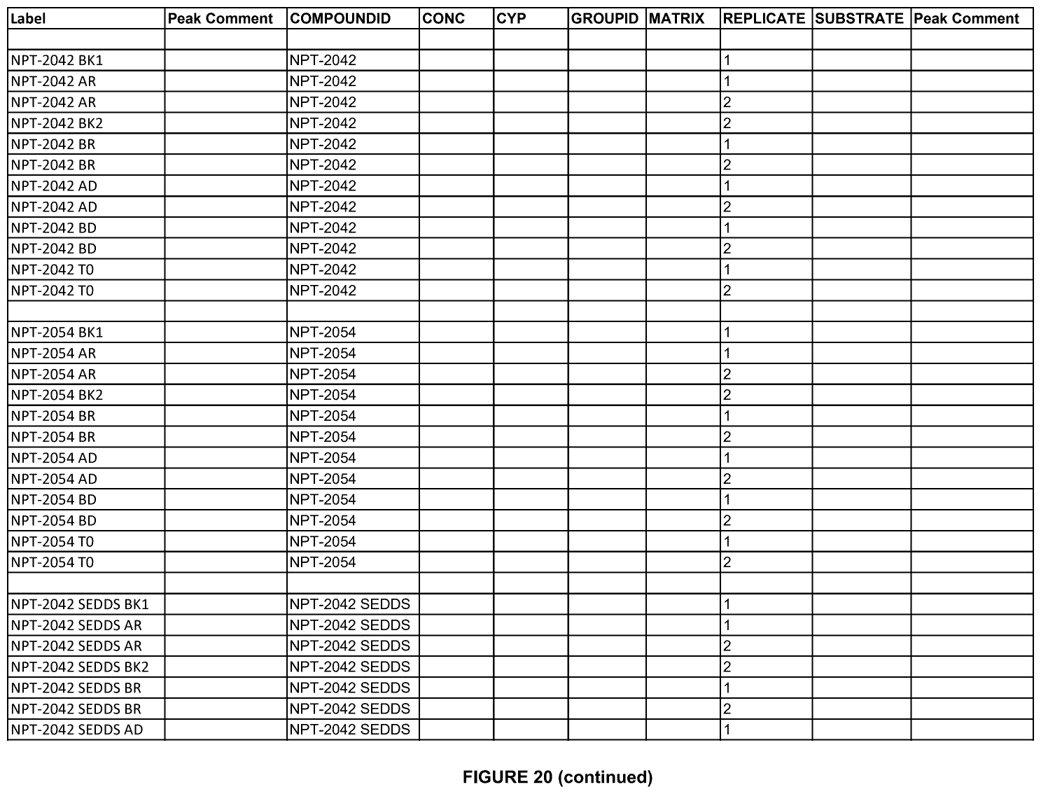

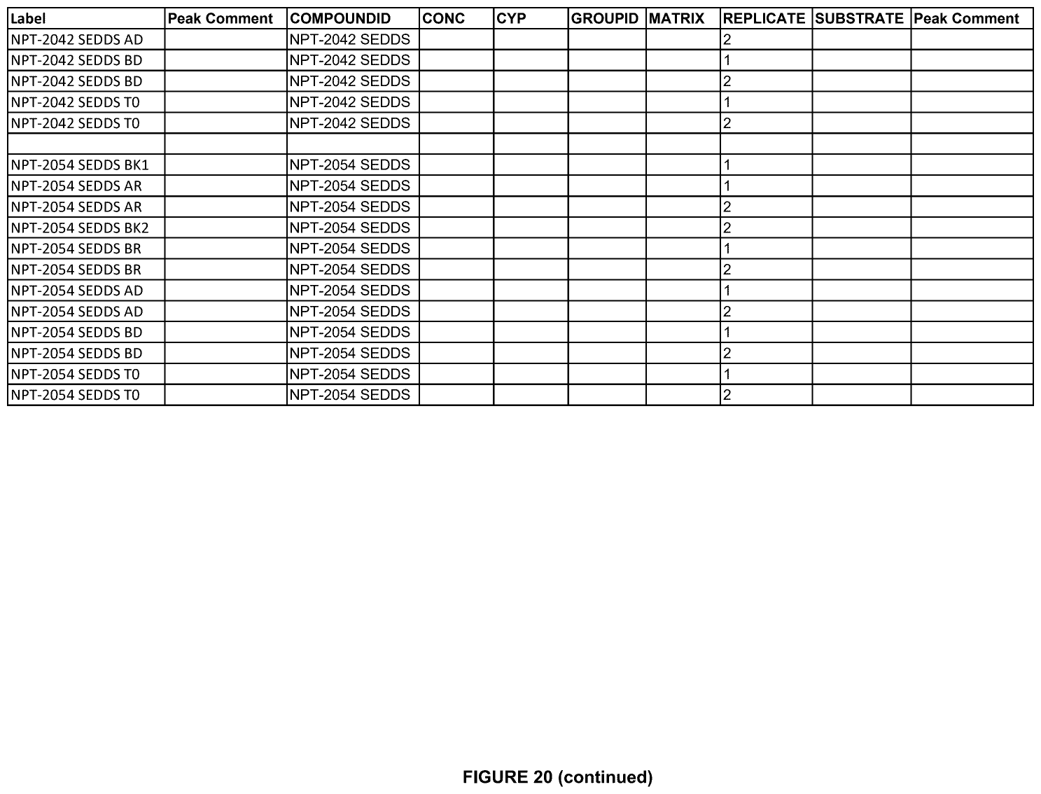

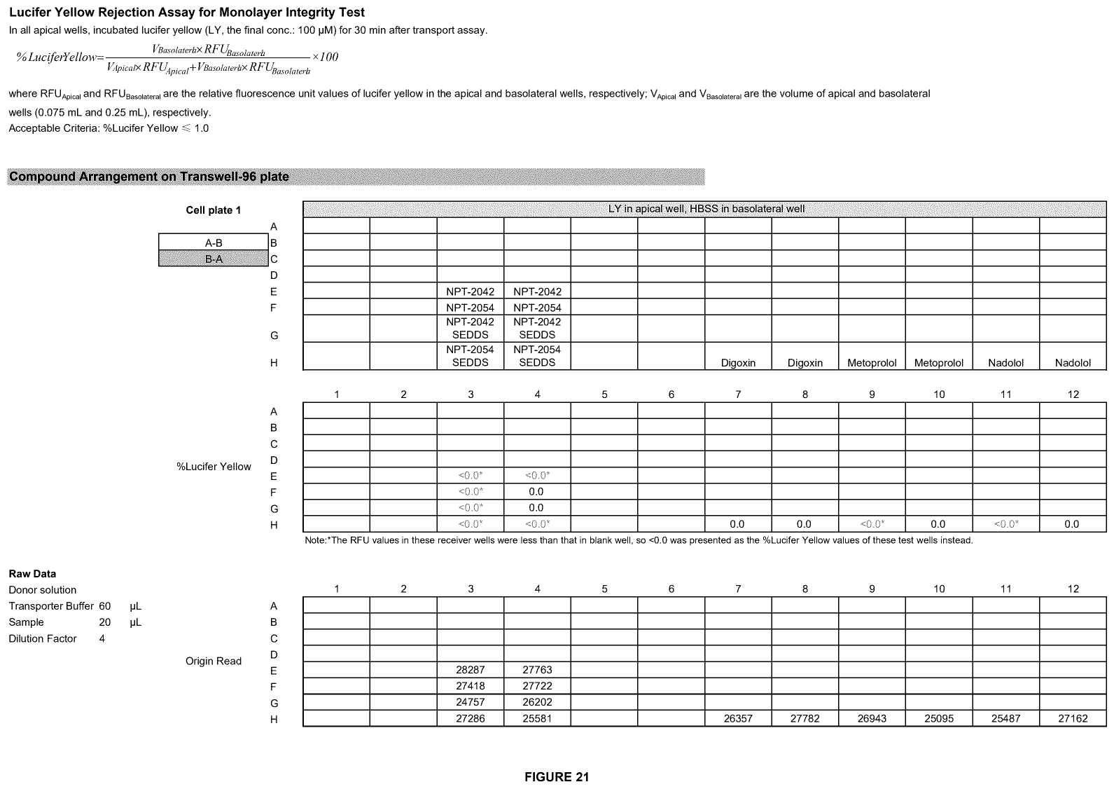

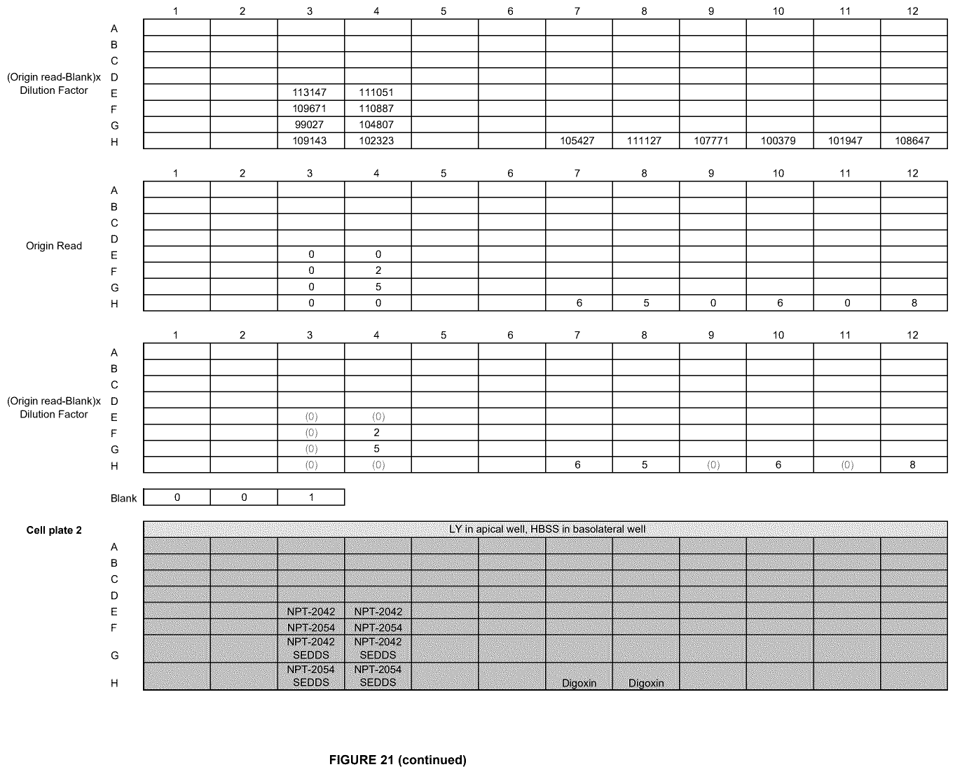

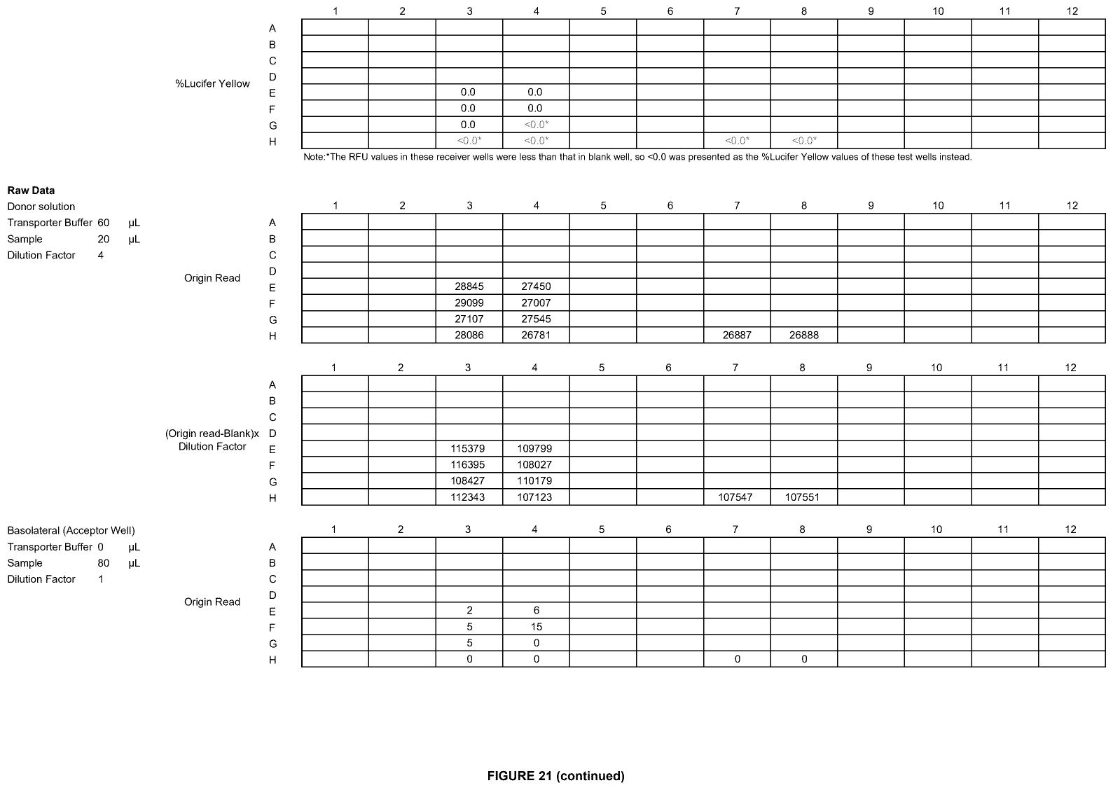

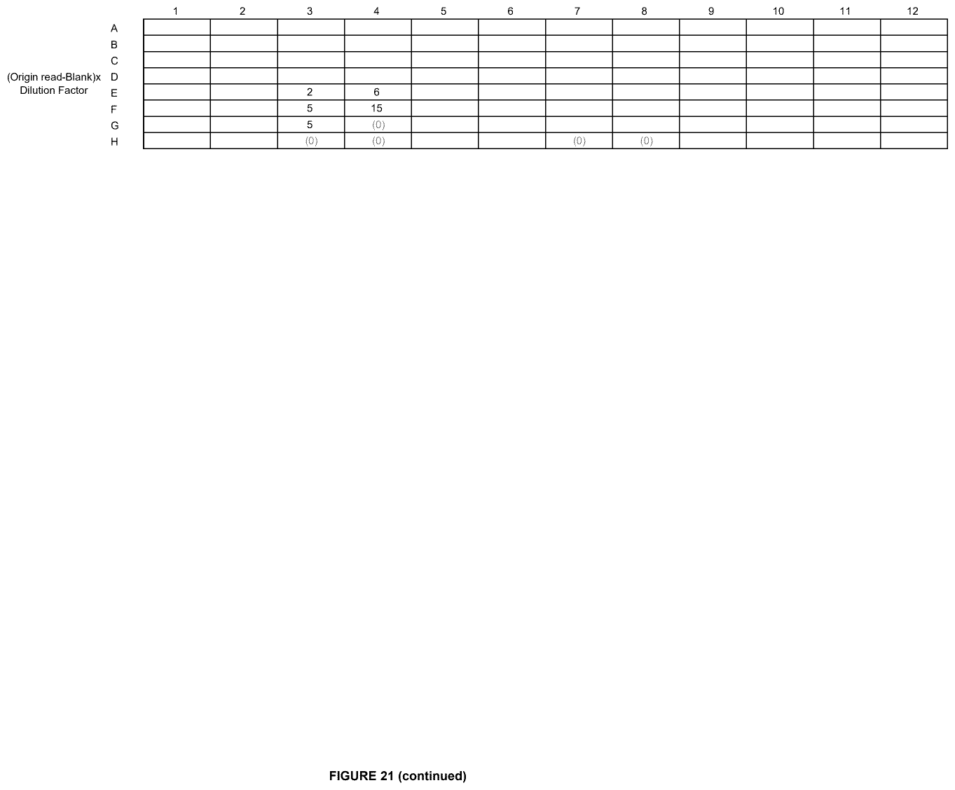

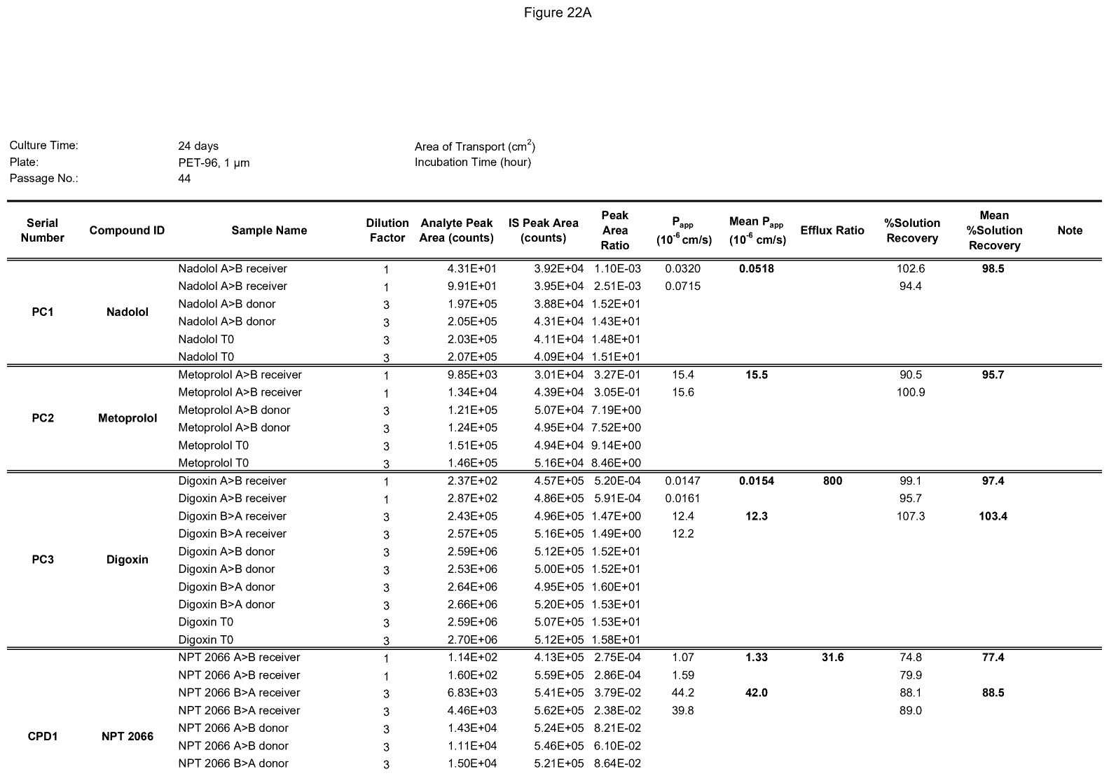

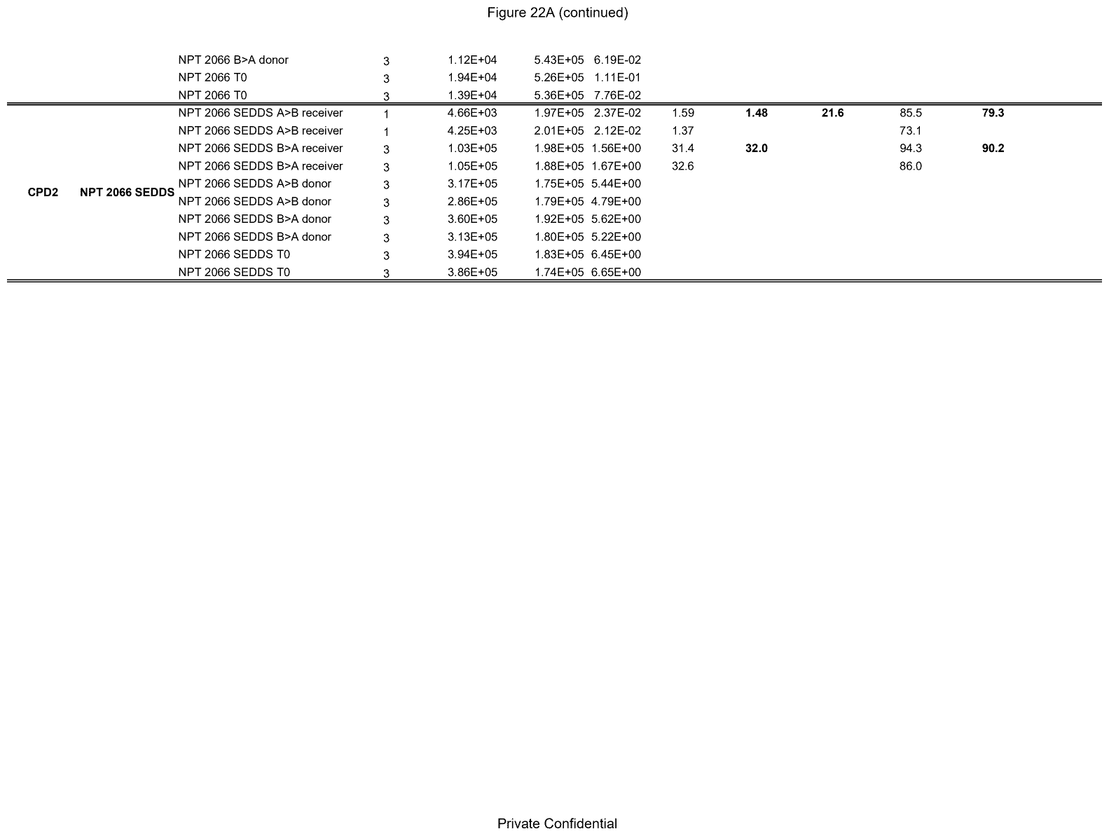

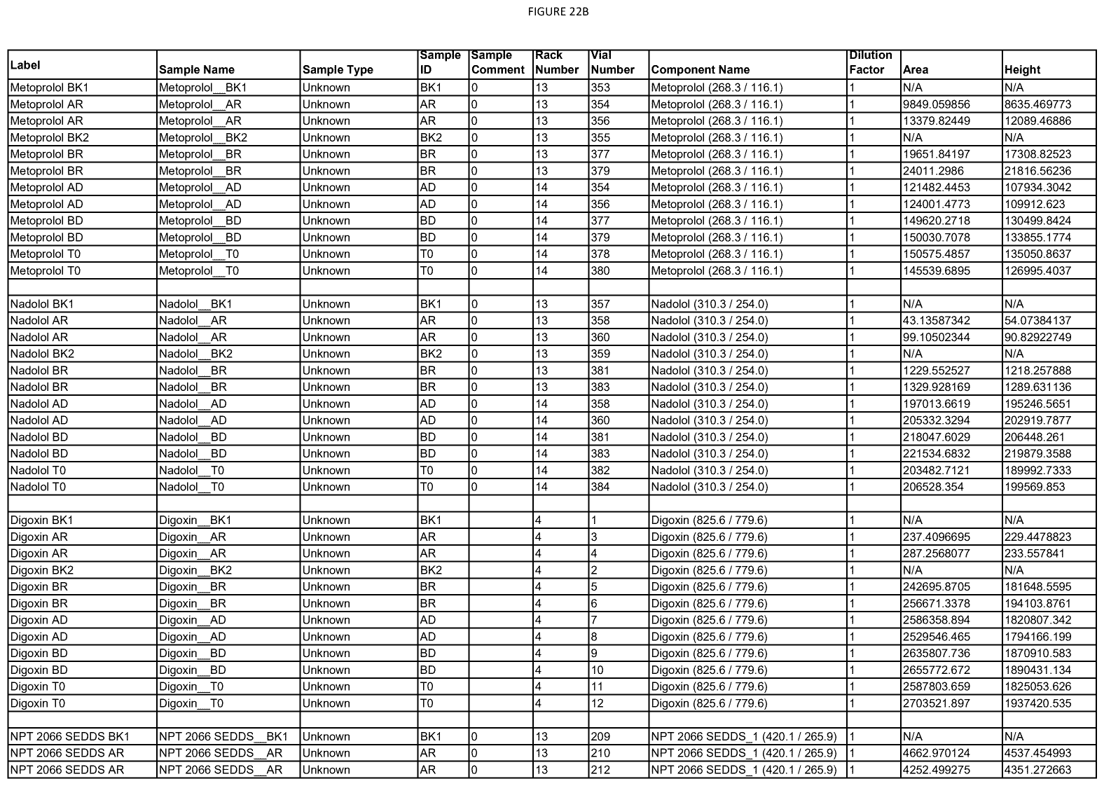

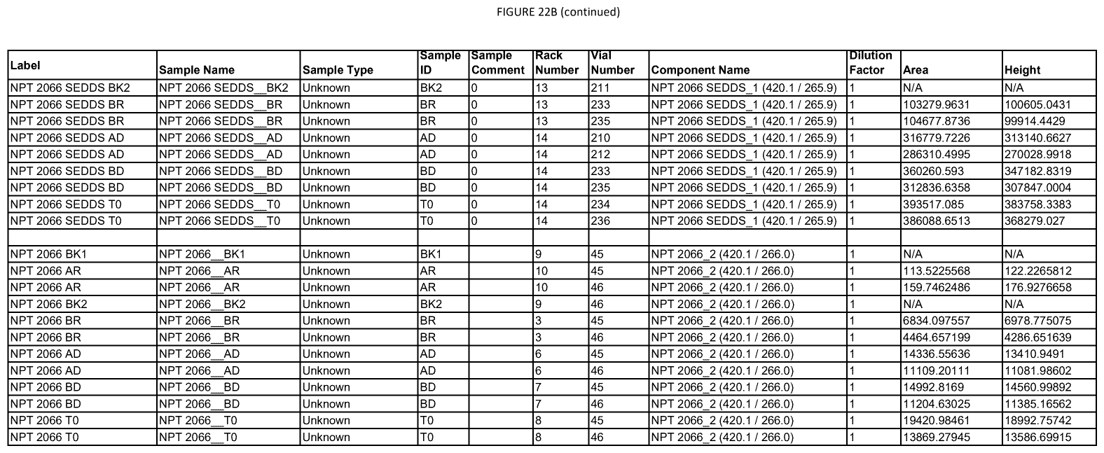

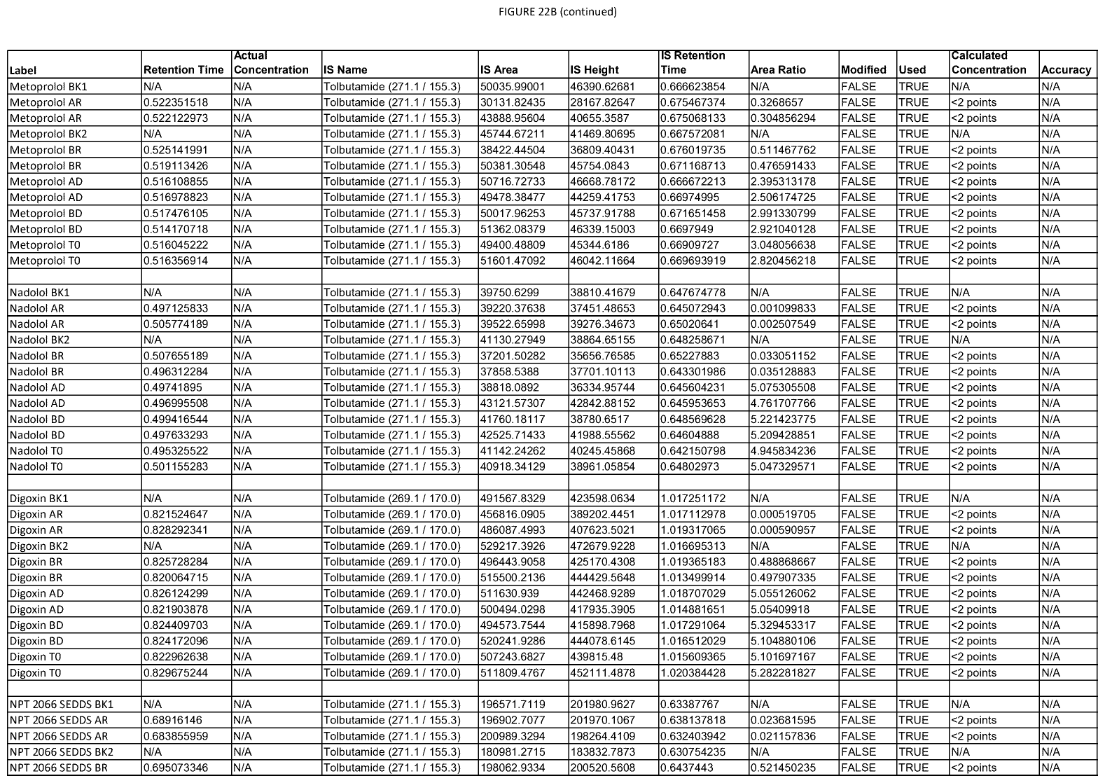

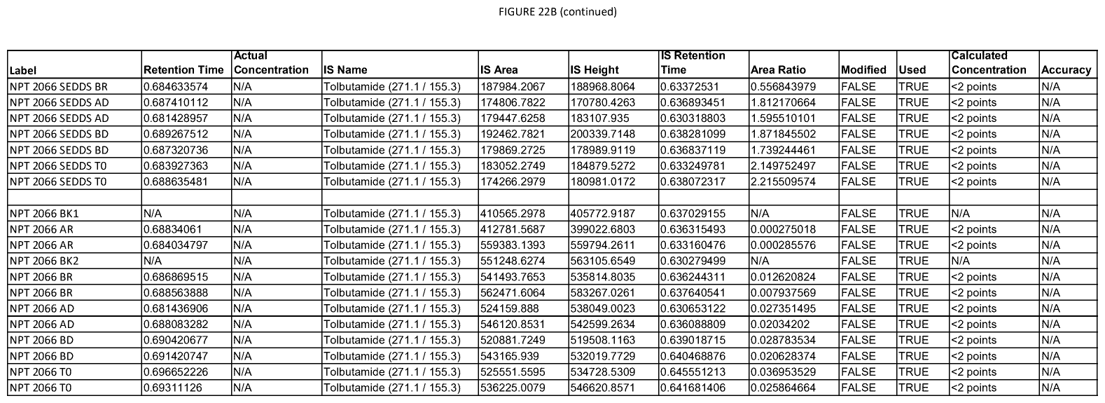

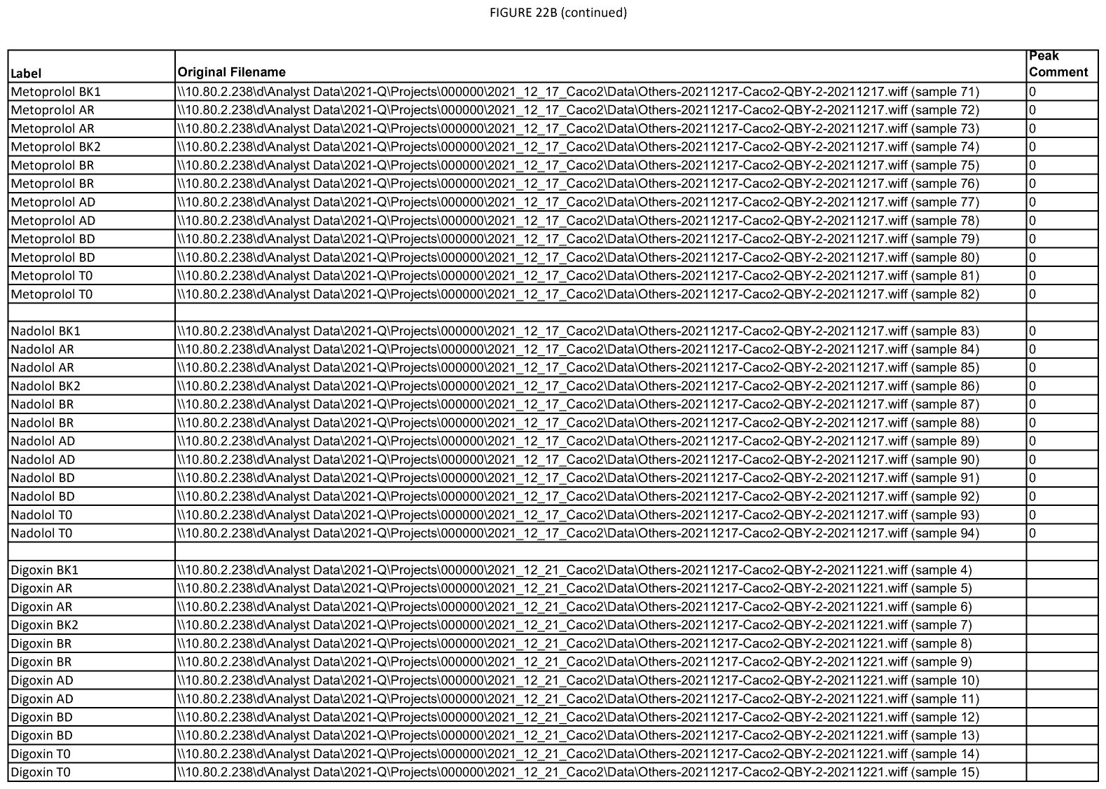

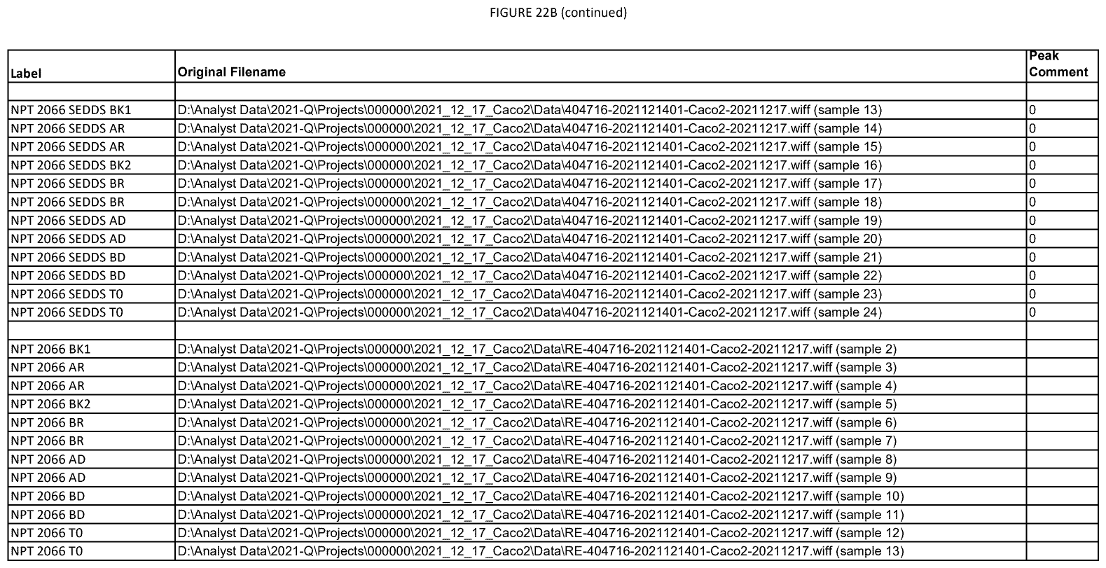

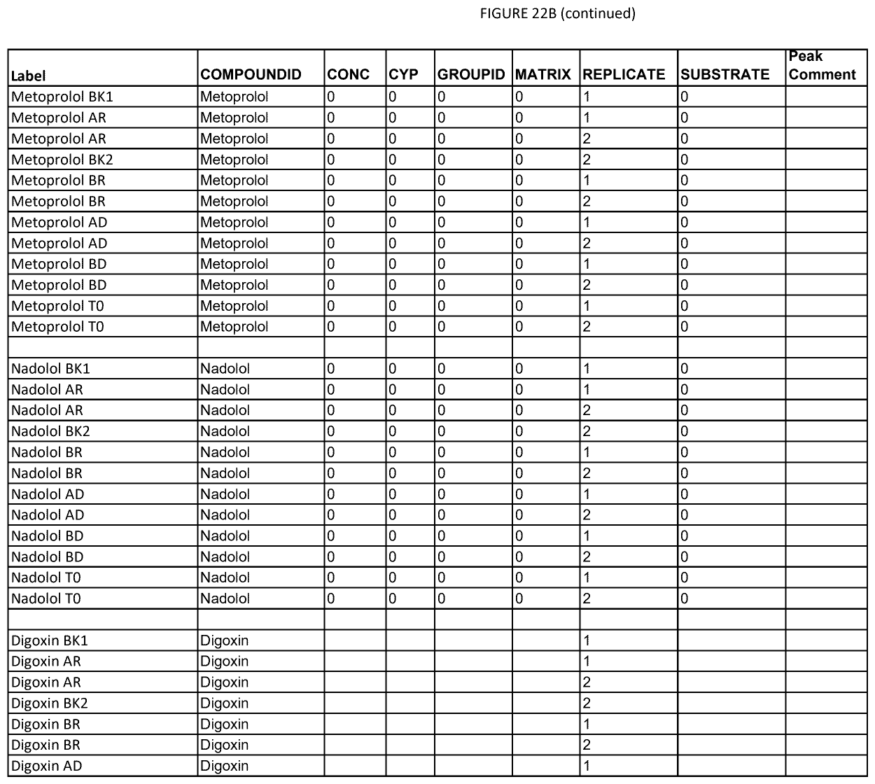

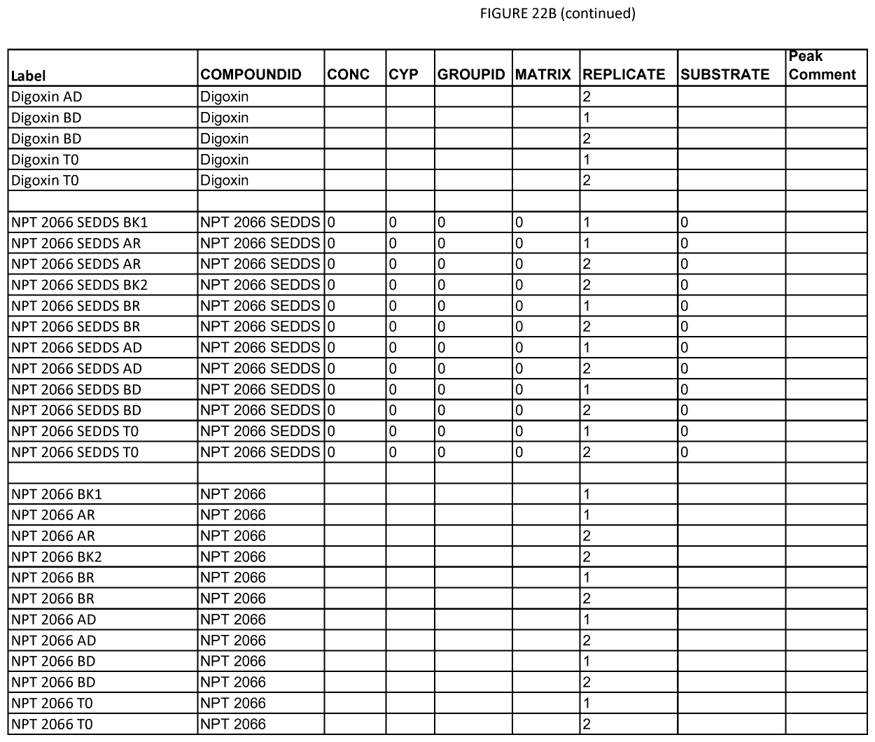

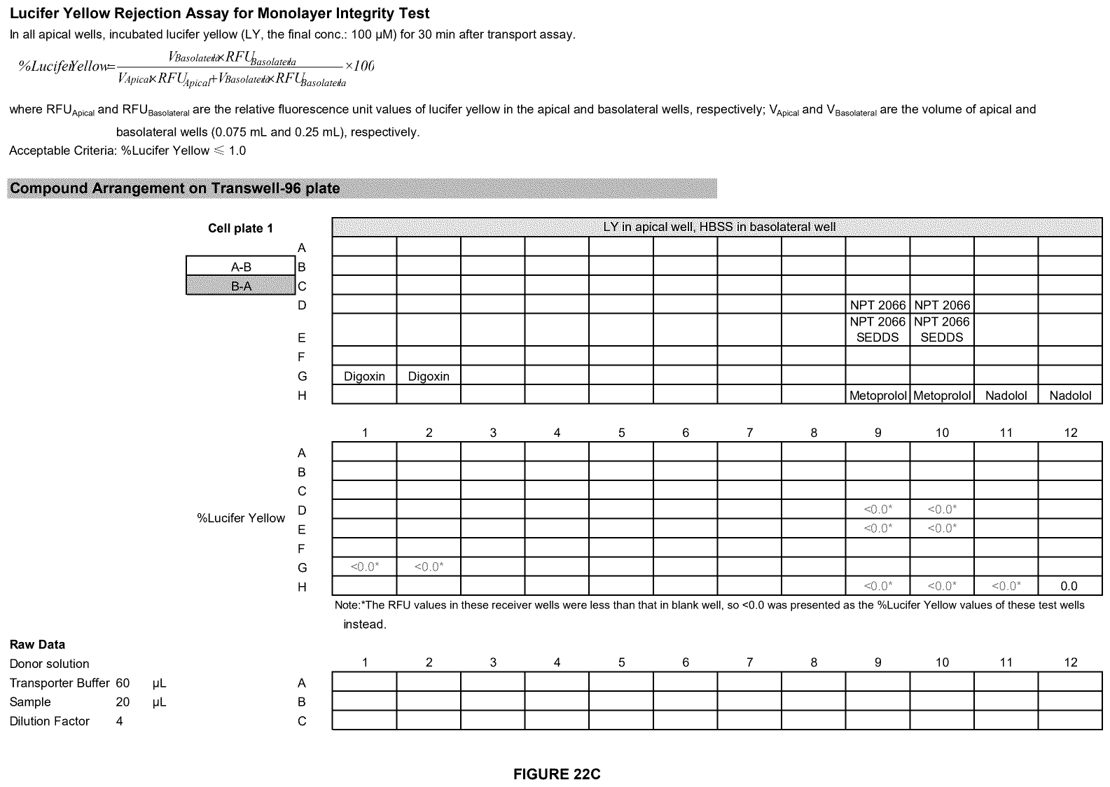

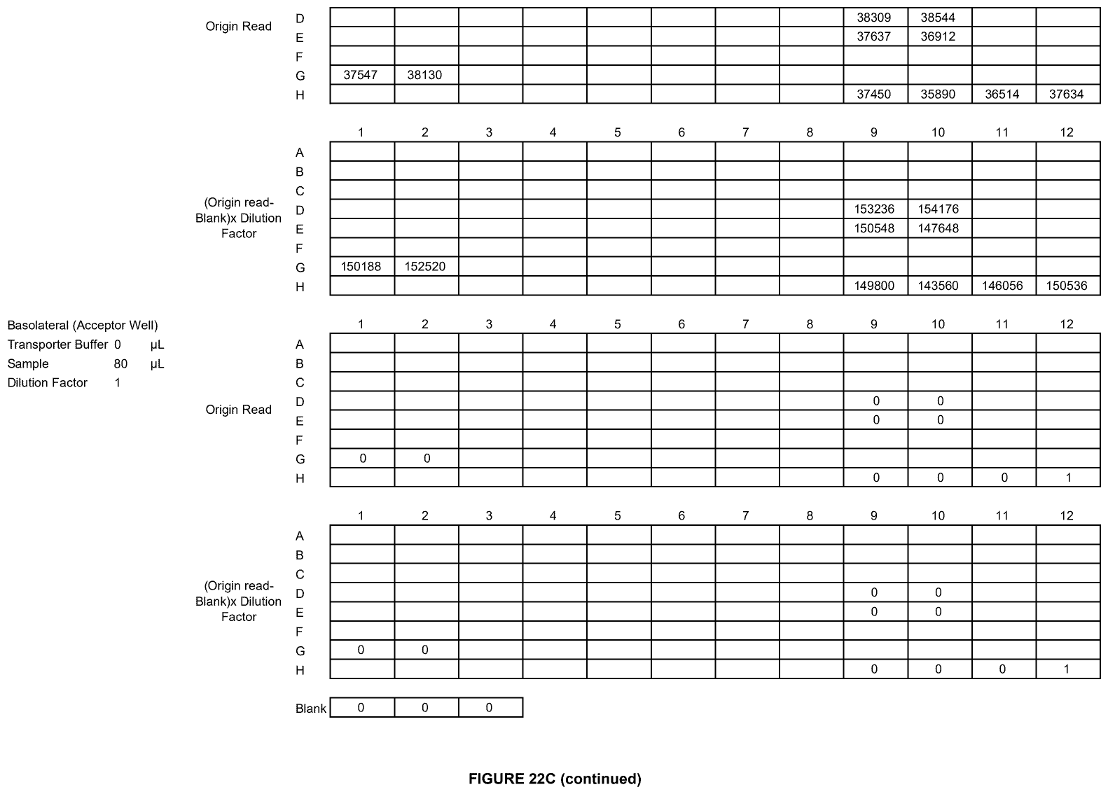

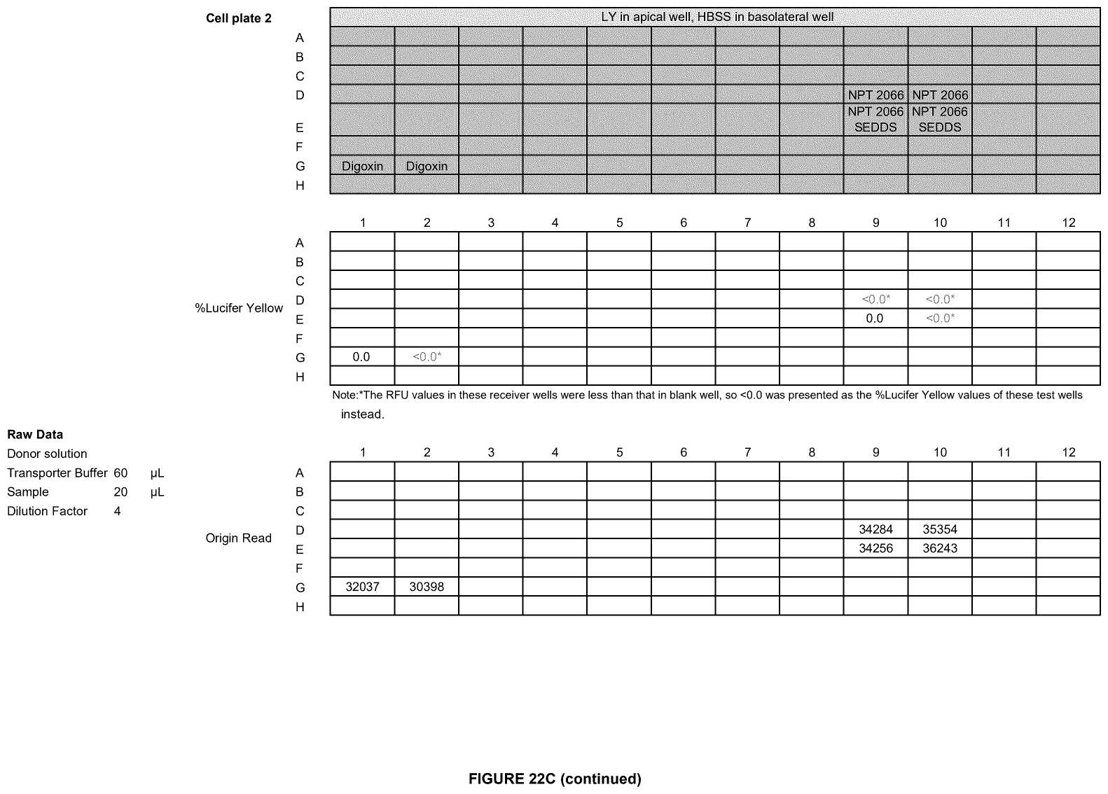

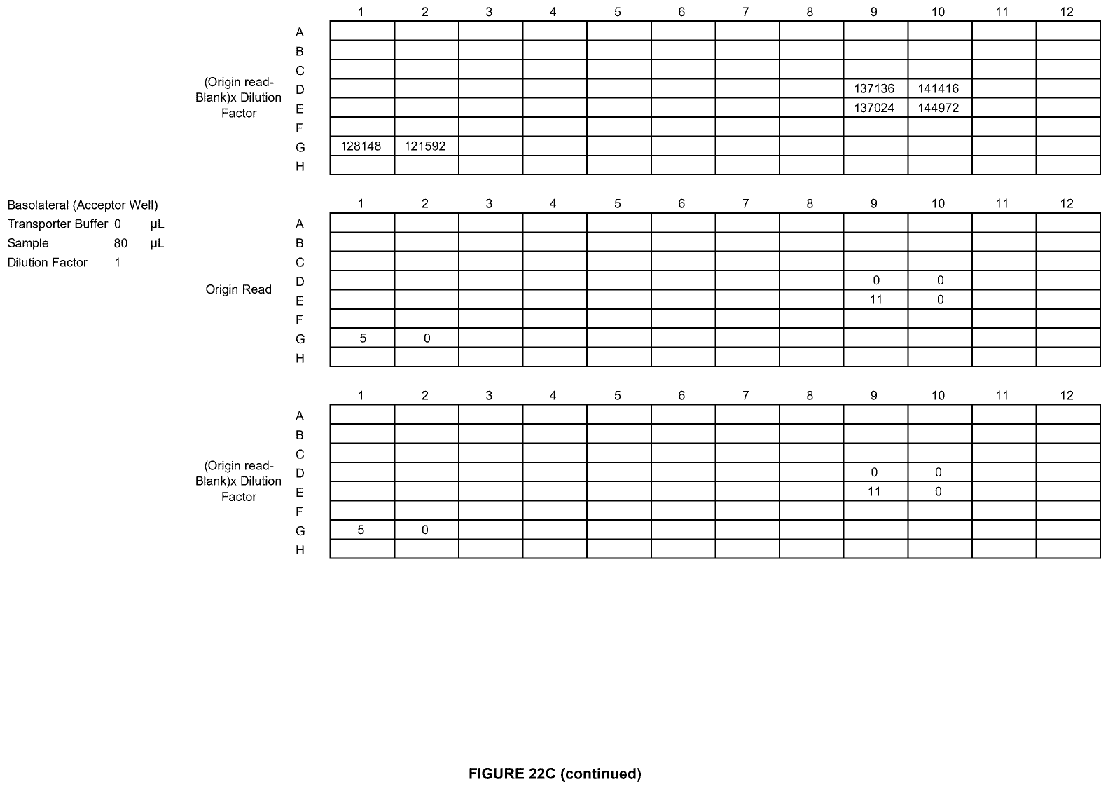

FIG. 19 represents a tabulated calculation for this Example 9. FIG. 20 is a tabulated depiction of LCMS data for the tested compounds. FIG. 21 is a tabulated depiction of the Lucifer Yellow Rejection Assay for Monolayer Integrity. FIGS. 22A, 22B, and 22C likewise are (A) a tabulated calculation of the Permeability of Test Compounds in Caco-2 Assay of Example 9; (B) a tabulated depiction of LCMS data from the Permeability of Test Compounds in Caco-2 Assay of Example 9; and (C) a tabulated depiction of the Lucifer Yellow Rejection Assay for Monolayer Integrity Test associated with the Permeability of Test Compounds in Caco-2 Assay of Example 9.

Based on the results of this assay, which is a standard test for predicting permeability of drugs in the intestine, one would not have supported advancing the present disclosure of a SEDDS formulation for the test compounds: Bumetanide Dibenzylamide and Bumetanide Morpholinoamide. Thus, this data supports the unexpected results of the present disclosure.

### Example 10: The SEDDS Formulation Increases Bioavailability of Bumetanide Amides

The SEDDS formulation described above increases the bioavailability of Bumeanide Amides including but not limited to Bumetanide Morpholinoamide and Bumetanide Diethylamide.

### Methodology

SEDDS-formulated and unformulated Bumetanide Morpholinoamide and Bumetanide Diethylamide were orally administered to rats. The experiment was conducted as described previously in regards to Bumetanide Dibenzylamide. Three male and three female rats at each time point for each of the SEDDS and non-SEDDS treatments were used. The female rats showed no concentrations of any drug, at any formulation or time. As this appeared to be experimental error, all female rats were removed from the data.

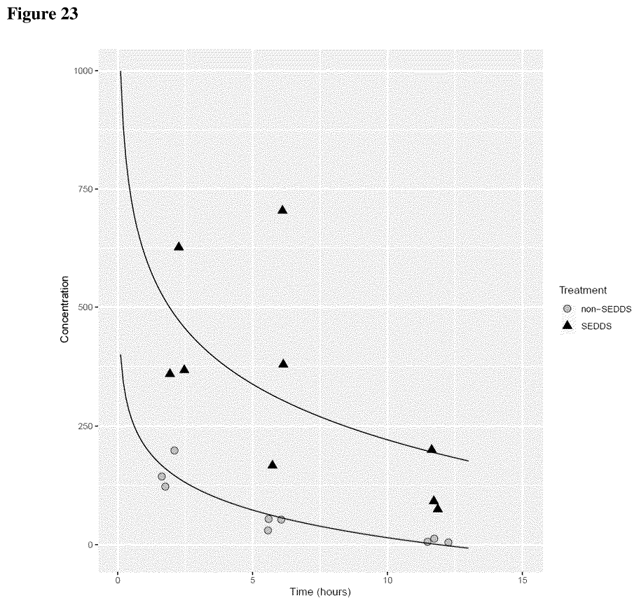

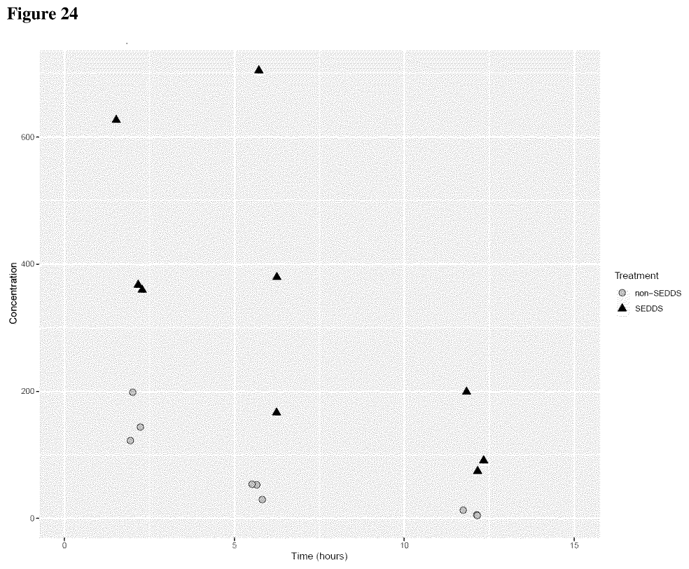

Blood concentration of the SEDDS formulated (black triangles) and unformulated (grey circles) Bumetanide Morpholinoamide at 2 hrs, 6 hrs, and 12 hrs post treatment is shown in FIG. 23. An increased concentration of SEDDS formulated Bumetanide Morpholinoamide was observed. However, there was no detectable concentration on blood by 24 hrs (data not shown). FIG. 24 shows an exponential fit to the data to predict what the blood concentration values of formulated and unformulated Bumetanide Morpholinoamide might have been at time points prior to 2 hrs.

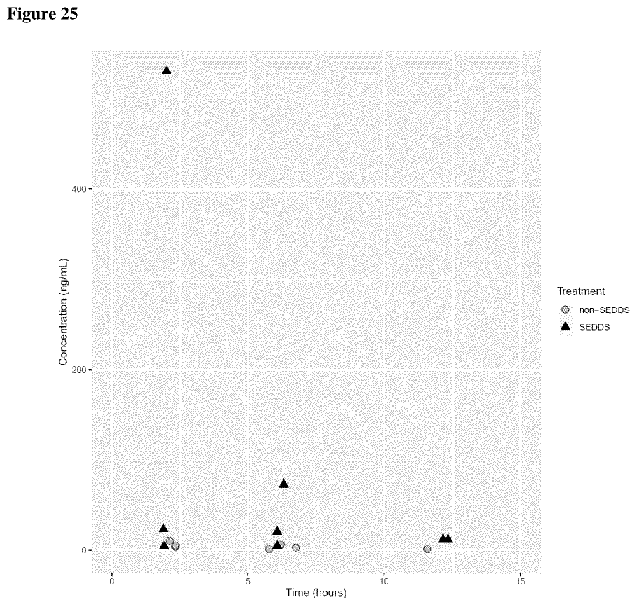

Blood concentration of the formulated (black triangles) and unformulated (grey circles) Bumetanide Diethylamide at 2 hrs, 6 hrs, and 12 hrs post treatment is shown in FIG. 25. Based on the data recorded, the doasage for both the may have been less than an effective amount. Additionally, there was outlier recorded. However, even if exclude that one outlier is excluded, the average blood concentration of SEDDs formulated is still higher that the blood concentration of unformulated Bumetanide Diethylamide at all time points.

Based on these observations, SEDDS formulation may be deemed to be an effective method of formulating and administering Bumetanide Amides.

Those skilled in the art to which the present disclosure pertains may make modifications resulting in other embodiments employing principles of the present disclosure without departing from its spirit or characteristics, particularly upon considering the foregoing teachings. Accordingly, the described embodiments are to be considered in all respects only as illustrative, and not restrictive, and the scope of the present disclosure is, therefore, indicated by the appended claims rather than by the foregoing description or drawings. Consequently, while the present disclosure has been described with reference to particular embodiments, modifications of structure, sequence, materials and the like apparent to those skilled in the art still fall within the scope as claimed.

1-109. (canceled)

110. A pharmaceutical composition comprising a Bumetanide Dibenzylamide and one or more solubilizers.

111. The composition of claim 110, wherein the solubilizer is selected from the group consisting of a surfactant, an oil, and a solvent.

112. The composition of claim 111, wherein the surfactant comprises Polyoxyl 35 Castor Oil.

113. The composition of claim 111, wherein the oil comprises maisine oil, soybean oil, or other vegetable oil.

114. The composition of claim 111, wherein the oil comprises short chain triglycerides, long chain triglycerides, and combinations thereof.

115. The composition of claim 111, wherein the solvent is selected from the group consisting of Ethanol, Propylene Glycol, Polyethylene Glycol 600, Polyethylene Glycol 3350, Oleyl Alcohol, and any combination thereof.

116. The composition of claim 111, wherein the composition comprises permeation enhancement.

117. The composition of claim 110, wherein the one or more solubilizers are selected from the group consisting of Caprylocaproyl Polyoxylglycerides, Phosphatidylcholine, Caprylic/Capric Triglyceride, Polyethylene glycol mono-, di- and tri-glycerides, Sorbitan Ester, and any combination thereof.

118. The composition of claim 110, comprising the Bumetanide Dibenzylamide in a self-emulsifying drug delivery system (SEDDS).

119. The composition of claim 118, wherein the SEDDS comprises an isotropic mixture of oils, surfactants, and co-solvents.

120. The composition of claim 110, wherein the Bumetanide Dibenzylamide is in an oral lymphatic targeted formulation.

121. The composition of claim 110, wherein the composition is comprised in a 20 mg soft gelatin capsule.

122. The composition of claim 110, wherein the gelatin capsule comprises about 1.75% w/w Bumetanide Dibenzylamide.

123. The composition of claim 110, comprising: about 1 to 2 w/w % Bumetanide Dibenzylamide, about 30 to 35 w/w % Polyoxyl 35 Castor Oil (Kolliphor EL), about 30 to 35 w/w % Glyceryl Monolinoleate (Maisine CC), about 30 to 35 w/w % Soybean Oil, about 0.05% to 0.16% Butylated hydroxyanisole, and about 2.5 to 5 w/w % Ethanol.

124. The composition of claim 110, comprising: about 1.75 w/w % Bumetanide Dibenzylamide, about 32.24 w/w % Polyoxyl 35 Castor Oil (Kolliphor EL), about 31.3 w/w % Glyceryl Monolinoleate (Maisine CC), about 31.3 w/w % Soybean Oil, and about 3.41 w/w % Ethanol 200 Proof.

125. The composition of claim 110, comprising: about 1.75 w/w % Bumetanide Dibenzylamide, about 32.24 w/w % Polyoxyl 35 Castor Oil (Kolliphor EL), about 31.3 w/w % Glyceryl Monolinoleate (Maisine CC), about 31.3 w/w % Soybean Oil, about 0.16% Butylated hydroxyanisole, and about 3.25 w/w % Ethanol 200 Proof.

126. A pharmaceutical composition comprising an analog of Bumetanide and one or more oral lymphatic targeting excipients.

127. The pharmaceutical composition of claim 126, wherein the analog of Bumetanide is an amide analog.

128. The pharmaceutical composition of claim 126, wherein the analog of Bumetanide is one or more of Bumetanide Dibenzylamide, Bumetanide Diethylamide, and Bumetanide Morpholinoamide.

129. A method for treating a patient in need comprising administering a pharmaceutical composition comprising about 20 mg/g of a Bumetanide analog and one or more solubilizers comprising short chain triglycerides, long chain triglycerides, or combinations thereof.
